SEQUENCE LISTING (101 sequences)

SEQ ID NO: 1 - protein, 368 aa, Gentiana rigescens
MALIYSTPSWVQAHTISIYHGNGSSFFPCYLSKNKAPVFLSNPCKKPNLGRSPLSICAILTKEESKIKKAHDFSFNFKDYMLEKADSVNKALEQAVSIREPLKIHESMRYSLLAGGKRVRPMLCIAACELFGGDESVAMPSACAVEMIHTMSLMHDDLPCMDNDDLRRGKPTNHKVYGEDVAVLAGDALLAFAFEHIATSTKGVTSERIVRVIGELAKCIGSEGLVAGQIVDVCSEGISDVGLQHLEFIHIHKTAALLEGSVAMGAILGGADDEEVSKLRKFARGIGLLFQVVDDILDVTKSSKELGKTAAKDLVADKVTYPKLIGIDKSREFAEKLNREAQDQLAGFDSEKAAPLIALANYIAYRDN

SEQ ID NO: 2 - protein, 372 aa, Swertia mussotii
MSLVNSTATSWLQAHTISNYYGGNGSNLSPYYLCHTFKNKLGPPISQKESTFRYSSFSICAILTKEESKIKKAHDFSSFNFEDYMIEKANSVNKALESAVSIREPLKIHESMRYSLLAGGKRIRPMLCIAACELFGGDESIAMPSACAVEMIHTMSLMHDDLPCMDNDDLRRGKPTNHKVFGEDVAVLAGDALLAFAFEHIATSTKGVSSDRIVRVIGELARFVGSEGLVAGQIVDVCSEGKSDVGLKHLEFIHIHKTAALLEGSVALGAILGGANDEQVLKLKKFARGIGLLFQVVDDILDVTKSSKELGKTAGKDLVADKVTYPKLIGIEKSREFADKLNREAQEQLSGFDPEKAAPLIALANYIAYRDN

SEQ ID NO: 3 - protein, 419 aa, Camptotheca acuminate
MLFYRGLSRISRTSLNHGWWLLSFRNEQQLVPSNNFHYPRYTAEKVLGCRETYSWASHTFHGVGHQIHHQSCTIDEEQLDPFSLVADELSVLANRLRSMVVAEVPKLASAAEYLFKMGVEGKRFRPTVLLLMATALNVPIPGPAPDRSVDSLSMELRTRQQCIAEITEMIHVASLLHDDVLDDADTRRGIGSLNFIMGNKLAVLGGDFLLSRACVALASLKNTEVVSLLATVVEHLVTGETMQMTTSSEQRCSMEYYLQKTYYKTASLISNSCKAVALLAGQTAEVSLLAYEYGKNLGLAYQLIDDVLDFIGTSTSLGKGSLSDIRHGIVTAPILYAIEEFPQLRAVVDEGFDKPANVDLALQYLGRSCGIQRTRELATKHANLASAAIDSLPESNDEDVQKSRRALVGLTHRVITRTK

SEQ ID NO: 4 - protein, 422 aa, Arabidopsis thaliana
MLFTRSVARISSKFLRNRSFYGSSQSLASHRFAIIPDQGHSCSDSPHKGYVCRTTYSLKSPVFGGFSHQLYHQSSSLVEEELDPFSLVADELSLLSNKLREMVLAEVPKLASAAEYFFKRGVQGKQFRSTILLLMATALNVRVPEALIGESTDIVTSELRVRQRGIAEITEMIHVASLLHDDVLDDADTRRGVGSLNVVMGNKMSVLAGDFLLSRACGALAALKNTEVVALLATAVEHLVTGETMEITSSTEQRYSMDYYMQKTYYKTASLISNSCKAVAVLTGQTAEVAVLAFEYGRNLGLAFQLIDDILDFTGTSASLGKGSLSDIRHGVITAPILFAMEEFPQLREVVDQVEKDPRNVDIALEYLGKSKGIQRARELAMEHANLAAAAIGSLPETDNEDVKRSRRALIDLTHRVITRNK

SEQ ID NO: 5 - protein, 321 aa, Arabidopsis thaliana
MVLAEVPKLASAAEYFFKRGVQGKQFRSTILLLMATALNVRVPEALIGESTDIVTSELRVRQRGIAEITEMIHVASLLHDDVLDDADTRRGVGSLNVVMGNKMSVLAGDFLLSRACGALAALKNTEVVALLATAVEHLVTGETMEITSSTEQRYSMDYYMQKTYYKTASLISNSCKAVAVLTGQTAEVAVLAFEYGRNLGLAFQLIDDILDFTGTSASLGKGSLSDIRHGVITAPILFAMEEFPQLREVVDQVEKDPRNVDIALEYLGKSKGIQRARELAMEHANLAAAAIGSLPETDNEDVKRSRRALIDLTHRVITRNK

SEQ ID NO: 6 - protein, 283 aa, Glycine max
MLGALLLNANFKIHFSLISCQARVPLPVKPAPLRMPSPHYPHWASLQADIEAHLKQTIPLKEPLEVFEPMLHLAFSAPRTTVPALCLAACELVGGHRQQAMAAASALLLNLANAHAHEHLTDGPMYGPNIELLTGDGIVPFGFELLARPDGPASASPERVLRVMIEISRAVGSVGLQDAQYVKKTLWDGGEEVQNVESMQRFVLEKRDGGLHACGAASGAILGGGSEDQIERLRNFGFHVGMMRGMLQMGFMEKHVQEERHLALKELQFFMDRDVHVISSFIY

SEQ ID NO: 7 - protein, 412 aa, Helianthus annuus
MSIYRAISRITRTASSYNRCRWFYSSAPHQQLSPYSGFRSSEQVLGCRVISPWFSRSFRSGGPQPQYEDDQEDPFSLVADELSIVANRLRSMVVAEVPKLASAAEYFFKMGVEGKRFRPTVILLMATALNNQISKPPSEGVVDMLSTEFRTRLQSIAEITEMIHVASLLHDDVLDDADTRRGIGSLNFVMGNKISVLAGDFLLSRACITLASLKNTEVVSLIATAVEHLVTGETMQMSSSAEQRSSMDYYLQKTYYKTASLISNSCKSIALLTGQTAEVAMLAYEYGKNLGLAFQLIDDVLDFTGTSSSLGKGSLSDIRHGIVTAPLLYAMEEFPELRSVVDRGLDNPANVDLALEYLGKSHGIQRTRELAAKHASLASAAIDSFPENDDEDVQRSRRALIELTHRVINRTK

SEQ ID NO: 8 - protein, 415 aa, Withania somnifera
MIFSRVLSQISRNRFSRCRWLFSLPPHQQLHHSNNIYASQKVLGCRVIHSWVSNALSGIGQQIHHQTSAVAEEQVDPFSLVADELSLLTNRLRSMVVAEVPKLASAAEYFFKMGVEGKRFRPTVLLLMATALNVQIPRSAPHVDVDSLSGDLRTRQQCIAEITEMIHVASLLHDDVLDDAETRRGIGSLNYVMGNKLAVLAGDFLLSRACVALASLKNTEVVSLLATVVEHLVTGETMQMTTSSDERCSMEYYMQKTYYKTASLISNSCKAIALLAGHTAEVSVLAFDYGKNLGLAFQLIDDVLDFTGTSATLGKGSLSDIRHGIVTAPILYAMEEFPQLRTLVDRGFDDPVNVEIALDYLGKSRGIQRTRELARKHASLASAAIDSLPESHDEEVQRSRRALVELTHRVITRTK

SEQ ID NO: 9 - protein, 342 aa, Selaginella moellendorffii
MAQLGRRLRDMVAAEVPKLASAAEYFFKLGVEGKRFRPMVLLLMSSSLTMVLPSAAAATSDEKNWRHHKLAEITEMIHVASLLHDDVLDHADTRRGIASLNFIMGNKLAVLAGDFLLARAAFSLSTLQNDEVVGLMSKVLEHLVAGEVMQWTVDAEKSSSMDYYLQKTFYKTASLIANSCKCIAILAGHPKEVAALAFDYGRHLGLAYQLVDDLLDFTGTKASLGKPALSDLREGIATAPVLYALEEHPALQELIDRKFKDPGDVDSALKMVLASSGIRKTKELAREHASKAADAVAGFPPTTSEKASLCRRALTELTEQVITRSNRGRMCCEAVNLSARFN

SEQ ID NO: 10 - protein, 421 aa, Paeonia lactiflora
MLYSRGFSRIPRNSLIRCCKWFLSSQQYHQQSFLSIKFQPPTDHTQKVLGCREIYSRGLLALHGIQHQSYHGGSSVIEERLDPFSLVADELSVIANRLRAMVVAKVPKLGSAAEYFFKIGVEGKRFRPTILLLMATALNVSIPGRAHAVLGDTLATELRTRQQCIAEITEMIHVASLLHDDVLDDADTRRGISSLNSVVGNKVAVLAGDFLLSRACVALASLRNTDVVILLATVVEHLVTGETMQMTTTSEQRCSMDYYMEKTYYKTASLISNSCKAIALLAGQTAEVAMLAFEYGKNLGLAFQLIDDVLDFTGTSASLGKGSLSDIRRGIVTAPILFAVEEFPQLRALVDRGFHDPKDVDIALDYLGKSCGIQKTRELATKHANLAAAAIDSLPESDDEEVVKSRRALVDLTQRVITRTK

SEQ ID NO: 11 - protein, 420 aa, Catharanthus roseus
MLFSRGLYRIARTSLNRSRLLYPLQSQSPELLQSFQFRSPIGSSQKVSGFRVIYSWVSSALANVGQQVQRQSNSVAEEPLDPFSLVADELSILANRLRSMVVAEVPKLASAAEYFFKLGVEGKRFRPTVLLLMATAIDAPISRTPPDTSLDTLSTELRLRQQTIAEITKMIHVASLLHDDVLDDAETRRGIGSLNFVMGNKLAVLAGDFLLSRACVALASLKNTEVVSLLATVVEHLVTGETMQMTTTSDQRCSMEYYMQKTYYMTASLISNSCKAIALLAGQTSEVAMLAYEYGKNLGLAFQLIDDVLDFTGTSASLGKGSLSDIRHGIVTAPILFAIEEFPELRAVVDEGFENPYNVDLALHYLGKSRGIQRTRELAIKHANLASDAIDSLPVTDDEHVLRSRRALVELTQRVITRRK

SEQ ID NO: 12 - protein, 462 aa, Nannochloropsis gaditana
MPAPRKVGLRRLRGLVQSCSTGFRGGVQPSLISSRTAISYVNRAVDHIYYSHASIGSTTNIVHRSIRSGWAKTAADASIDVIVNAVTRPEIDEPTVKVAEPRRAIIKADQAGELEEDLALDLQRKPRLDLLAGWAGAARGVDPFKIVESDMRSLSAGIKSLLGSDHPVLEACAKYFFELDGGKKIRPTMVLLISRAVAAHAPAQGVNGSRAFTSTSESSTPLPSQKRLAEITEMIHTASLFHDDVIDEADERRGVPSINKIYGNKMAILAGDFLLARASVSLARLRNIEVVELLSTVIEHLVKGEVMQSRPQALVDGSGTGENGQAALEYYLHKNFYKTGSLMANSCRAAVLLAGGGDALQNQAFAYGRHVGLAFQLVDDVLDFEQTSETLGKPALNDLRQGLATAPVLLAARTFPDEVGDMVKRKFASEGDVERVREMAFFSIAMTSPRPRYNSSYLGTLL

SEQ ID NO: 13 - protein, 424 aa, Salvia miltiorrhiza
MISVRGLARLARSGYARRRWVYSSLGCSGSAPLQLEHSSHFRNPIQSSREVLGCRVIYSWVSNAISTVGQQVHLQSSSAVEEQLDPFSLVADELSILADRLRSMVVAEVPKLASAAEYFFKFGVEGKRFRPTVLLLMATALDLPIARQTSEVAVNTLSTELRTRQQCVAEITEMIHVASLLHDDVLDDADTRRGIGSLNYVMGNKLAVLAGDFLLSRACVALASLKNTEVVTLIAQVVEHLVTGETMQMTTTSEQRCSMEYYMEKTYYKTASLICNSCKSIALIAGQTAEVSNLAYEYGKNLGLAFQIIDDVLDFTGTSASLGKGSLSDIRHGIVTAPILFAIEEYPELRKIVDQGFEKSSNVDRALEILSKSSGIQRARELAAKHARLASAAIDALPENEDEVVQRSMRALVELTHIVITRTK

SEQ ID NO: 14 - protein, 321 aa, Vitis vinifera
  misc_feature (26)..(26): Xaa can be any naturally occurring amino acid
  misc_feature (209)..(209): Xaa can be any naturally occurring amino acid
MVVAEVPKLASAAEYFFKMGVEGKRXRPTVLLLMATALNVPLPRPALAEVPETLSTELRTRQQCIAEITEMIHVASLLHDDVLDDAETRRGIGSLNIMMGNKVAVLAGDFLLSRACVALASLKNTEVVSLLATVVEHLVTGETMQMTSTSEQRVSMEYYLQKTYYKTASLISNSCKAIALLAGQTAEVSMLAFEYGKNLGLAFQLIDDXLDFTGTSASLGKGSLSDIRHGIITAPILFAIEEFPQLDAVVKRGLDNPADIDLALDYLGRSRGIQRTRELAMKHANLAAEAIDSLPESGDEDVLRSRRALIDLTHRVITRTK

SEQ ID NO: 15 - protein, 416 aa, Ips pini
MFKLAQRLPKSVSSLGSQLSKNAPNQLAAATTSQLINTPGIRHKSRSSAVPSSLSKSMYDHNEEMKAAMKYMDEIYPEVMGQIEKVPQYEEIKPILVRLREAIDYTVPYGKRFKGVHIVSHFKLLADPKFITPENVKLSGVLGWCAEIIQAYFCMLDDIMDDSDTRRGKPTWYKLPGIGLNAVTDVCLMEMFTFELLKRYFPKHPSYADIHEILRNLLFLTHMGQGYDFTFIDPVTRKINFNDFTEENYTKLCRYKIIFSTFHNTLELTSAMANVYDPKKIKQLDPVLMRIGMMHQSQNDFKDLYRDQGEVLKQAEKSVLGTDIKTGQLTWFAQKALSICNDRQRKIIMDNYGKEDNKNSEAVREVYEELDLKGKFMEFEEESFEWLKKEIPKINNGIPHKVFQDYTYGVFKRRPE

SEQ ID NO: 16 - protein, 416 aa, Quercus robur
MLFSRISRIRRPGSNGFRWFLSHKTHLQFLNPPAYSYSSTHKVLGCREIFSWGLPALHGFRHNIHHQSSSIVEEQNDPFSLVADELSMVANRLRSMVVTEVPKLASAAEYFFKMGVEGKRFRPTVLLLMATAMNISILEPSLRGPGDALTTELRARQQRIAEITEMIHVASLLHDDVLDDADTRRGIGSLNFVMGNKLAVLAGDFLLSRACVALASLKNTEVVSLLAKVVEHLVTGETMQMTTTCEQRCSMEYYMQKTYYKTASLISNSCKAIALLGGQTSEVAMLAYEYGKNLGLAYQLIDDVLDFTGTSASLGKGSLSDIRHGIITAPILFAMEEFPQLREVVDRGFDDPANVDVALDYLGKSRGIQRARELAKKHANIAAEAIDSLPESNDEDVRKSRRALLDLTERVITRTK

SEQ ID NO: 17 - protein, 321 aa, Citrus sinensis
MVIAEVPKLASAAEYFFKMGVEGKRFRPTVLLLMATALNVRVPEPLHDGVEDASATELRTRQQCIAEITEMIHVASLLHDDVLDDADTRRGIGSLNFVMGNKLAVLAGDFLLSRACVALASLKNTEVVTLLATVVEHLVTGETMQMTTSSDQRCSMDYYMQKTYYKTASLISNSCKAIALLAGQTAEVAILAFDYGKNLGLAYQLIDDVLDFTGTSASLGKGSLSDIRHGIITAPILFAMEEFPQLRTVVEQGFEDSSNVDIALEYLGKSRGIQKTRELAVKHANLAAAAIDSLPENNDEDVTKSRRALLDLTHRVITRNK

SEQ ID NO: 18 - protein, 394 aa, Cannabis sativa
MHRVSLLCSFSQNQKASIFVKTKKMSTVNLTWVQTCSMFNQGGRSRSLSTFNLNLYHPLKKTPFSIQTPKQKRPTSPFSSISAVLTEQEAVKEGDEEKSIFNFKSYMVQKANSVNQALDSAVLLRDPIMIHESMRYSLLAGGKRVRPMLCLSACELVGGKESVAMPAACAVEMIHTMSLIHDDLPCMDNDDLRRGKPTNHKVFGEDVAVLAGDALLAFAFEHMAVSTVGVPAAKIVRAIGELAKSIGSEGLVAGQVVDIDSEGLANVGLEQLEFIHLHKTGALLEASVVLGAILGGGTDEEVEKLRSFARCIGLLFQVVDDILDVTKSSQELGKTAGKDLVADKVTYPRLMGIDKSREFAEQLNTEAKQHLSGFDPIKAAPLIALANYIAYRQN

SEQ ID NO: 19 - protein, 380 aa, Morus alba
MSCVNLSTWVQTCSLFNQAGGRSRLSSSSALNNLFHPLKNNFPVPLSSIPKRHRPSPSSSLSTVSAVLTQQETETVTEVLEEEKAPFNFKAYMIQKANSVNQALDDAVSLREPQTIHEAMRYSLLAGGKRVRPVLCLTACELVGGDESVAMPAALAVEMIHTMSLIHDDLPCMDNDDLRRGKPTNHKVFGEDVAVLAGDALLAFAFEHIAVSTAGVTPSRIVRAIGELAKSIGTEGLVAGQVVDIDSEGSDDAGLEKLEFIHIHKTAALLEASVVLGAILGGGTDDEVEKLRSFARCIGLLFQVVDDILDVTKSSQELGKTAGKDLVADKVTYPKLIGIEKSKEFAAKLNKEAQEQLSGFDPHKAAPLIALANYIANRQN

SEQ ID NO: 20 - protein, 302 aa, Alcanivorax borkumensis SK2
MSSKATREFAALNQLTDTAKARLEQALDHYLPAHSAASRLSHAMRYAALSGGKRIRPLLVYGAAQLAGAPLAKADVPAVAVELIHAYSLVHDDLPAMDDDDLRRGQPTCHKAFDEATAILAGDTLHTRAFELLACHGDYRDGSRISLIQHLCQAAGVDGMAAGQMQDMLAQGQQQTVAALEEMHYLKTGRLITASLQLGYFVAEKDDPSLLANLTEFGDAIGLAFQIQDDILDVTAATEQLGKPSGSDEKLQKSTFPSLLGLEQSQQRARQLCDQAQQTLAGYGPRALPLQQLAQYIITRNH

SEQ ID NO: 21 - protein, 312 aa, Chlorella variabilis
MGQVSAPVVEDMDICRQNLLNVVGERHPMLLAAANQIFSAGGKRLRPLIVLLVARATFPLTGLSDITERHRRLAEISEMLHTASLVHDDVLDECDVRRGKETVNSLYGTRVAVLAGDFLFAQSSWFLANLDNMEVIKLISQVIADFADGEISQAASLFDAYIDLRRYLDKSFWKTASLIAASCRSAAVFSDCDTEARPPNRSCSLPPRLPPPRRVALPAHLAGRCPWPPLLRRVQDEMVGDGLLQLIQGRFKEEGSLQRALELVSLGGGIDKARTLAREQGDLALASLACLPDTPAKRSLELMVDLVLERLY

SEQ ID NO: 22 - protein, 420 aa, Ips confuses
MFKLAQRLPKSVGSLGNQLSKVSNAPNQLMSQMVPVTFQVMNTPIRHKSKSSAVPSSLSKSMYEHNEEMKDAMKYMDEIYSEVMGQIEKVPQYEEVKPILVRLRDAIDYTVPYGKRFKGVHIVSHFKLLADPKFITPENVKLSGVLGWCAEIIQAYFCMLDDIMDDSDTRRGKPTWYKLPGIGLNAVTDVCLMEMFTFELLKRYFFQHPSCADIHEIFRNLLFLTHMGQGCDFTFIDPVTRKINFKEFTEENYTKLCRYKIIFSTFHNTLELTSAMANVYDPKKIQELDPVLMRIGMMHQSQNDFKDLYRDQGEVLKQVEKSVLGTDIRTGQLTWFAQKALSICNDRQRKIIMDNYGKEDTKHSEAVREVYEELDLKGKFMEFEEESFQWLKKEIPKINNGVPHKIFQDYTYGVFKRRPE

SEQ ID NO: 23 - protein, 427 aa, Picea glauca
MYTRCILKDKYSRFNLRRKFFTSTKSINALNGLPDSRNPRGESNGISQFKIQQVFPCKEYIWIDRHKFHDVGFQAQHKRSITDEEQVDPFSLVADELSILANRLRSMILTEIPKLGTAAEYFFKLGVEGKRFRPMVLLLMASSLTIGIPEVAADCLRKGLDEEQRLRQQRIAEITEMIHVASLLHDDVLDDADTRRGVGSLNFVMGNKLAVLAGDFLLSRASVALASLKNTEVVELLSKVLEHLVTGEIMQMTNTNEQRCSMEYYMQKTFYKTASLMANSCKAIALIAGQPAEVCMLAYDYGRNLGLAYQLVDDVLDFTGTTASLGKGSLSDIRQGIVTAPILFALEEFPQLHDVINRKFKKPGDIDLALEFLGKSDGIRKAKQLAAQHAGFATFSVESFPPSESEYVKLCRKALIDLSEKVITRTK

SEQ ID NO: 24 - protein, 429 aa, Dendroctonus armandi
MFSMKVCRNRSCREFLREARRTISKTSTDKNSDAISRAQDHKLNVESDSNGSYSRWKKQMHHNNIRALSTIQQSMVRPVQSSALVTKEQSRDFMALFPDLVRELTEVGRSQELPDVMRRFARVLQYNTPTGKKNRGLIVLSTYRMLEDPEKLTPENIRLASILGWCVEMVHAYFLILDDIMDGSETRRGALCWYRQSGIGLSAINDAIMMENAVYLLLKRHLKDHPMYVPMMELFHEGTIKTTLGQSLDAMCLDTNGKPKLDMFTMSRYTSIVKYKTAYYSFQMPVAIAMYLAGMSDEEQHRQAKTILMEMGQFFQIQDDFLDCFGDPTVTGKVGTDIQDGKCSWLAVVALQRASAAQRKIMEEYYGRPEPESVAQIKNLYVDLCLPNTYAIYEEESFNIIKTHIQQISKGLRHDLFFKIMEKIYKREC

SEQ ID NO: 25 - protein, 301 aa, Medicago sativa
MATTTSHLTNVKSTVHFSCISNQHRSHLTTKLKPTTVRMSMTQTPYWASLHADVEAHLKQTITIKEPLLVFEPMHHLIFTAPKTTVPALCLAACELVGGQRQEAISAASALLLMEAATYTHEHLPLSDRPGPKPGPMIDHVYGPNVELLTGDGIVPFGFELLARSDGGENSERILKVMVEISRAVGSGGGVIDAQYMKTLGGGSDGDEICHVEEIRRVVEKYEGRLHSCGAVCGGVLGGGCEEEIERLRKFGFYVGIIQGMIKWGFKEDHKEVVEARNLAIQELKFFKDKEVDAIKTFLNI

SEQ ID NO: 26 - protein, 720 aa, Cannabis sativa AAE1
MGKNYKSLDSVVASDFIALGITSEVAETLHGRLAEIVCNYGAATPQTWINIANHILSPDLPFSLHQMLFYGCYKDFGPAPPAWIPDPEKVKSTNLGALLEKRGKEFLGVKYKDPISSFSHFQEFSVRNPEVYWRTVLMDEMKISFSKDPECILRRDDINNPGGSEWLPGGYLNSAKNCLNVNSNKKLNDTMIVWRDEGNDDLPLNKLTLDQLRKRVWLVGYALEEMGLEKGCAIAIDMPMHVDAVVIYLAIVLAGYVVVSIADSFSAPEISTRLRLSKAKAIFTQDHIIRGKKRIPLYSRVVEAKSPMAIVIPCSGSNIGAELRDGDISWDYFLERAKEFKNCEFTAREQPVDAYTNILFSSGTTGEPKAIPWTQATPLKAAADGWSHLDIRKGDVIVWPTNLGWMMGPWLVYASLLNGASIALYNGSPLVSGFAKFVQDAKVTMLGVVPSIVRSWKSTNCVSGYDWSTIRCFSSSGEASNVDEYLWLMGRANYKPVIEMCGGTEIGGAFSAGSFLQAQSLSSFSSQCMGCTLYILDKNGYPMPKNKPGIGELALGPVMFGASKTLLNGNHHDVYFKGMPTLNGEVLRRHGDIFELTSNGYYHAHGRADDTMNIGGIKISSIEIERVCNEVDDRVFETTAIGVPPLGGGPEQLVIFFVLKDSNDTTIDLNQLRLSFNLGLQKKLNPLFKVTRVVPLSSLPRTATNKIMRRVLRQQFSHFE

SEQ ID NO: 27 - protein, 543 aa, Cannabis sativa AAE3
MEKSGYGRDGIYRSLRPPLHLPNNNNLSMVSFLFRNSSSYPQKPALIDSETNQILSFSHFKSTVIKVSHGFLNLGIKKNDVVLIYAPNSIHFPVCFLGIIASGAIATTSNPLYTVSELSKQVKDSNPKLIITVPQLLEKVKGFNLPTILIGPDSEQESSSDKVMTFNDLVNLGGSSGSEFPIVDDFKQSDTAALLYSSGTTGMSKGVVLTHKNFIASSLMVTMEQDLVGEMDNVFLCFLPMFHVFGLAIITYAQLQRGNTVISMARFDLEKMLKDVEKYKVTHLWVVPPVILALSKNSMVKKFNLSSIKYIGSGAAPLGKDLMEECSKVVPYGIVAQGYGMTETCGIVSMEDIRGGKRNSGSAGMLASGVEAQIVSVDTLKPLPPNQLGEIWVKGPNMMQGYFNNPQATKLTIDKKGWVHTGDLGYFDEDGHLYVVDRIKELIKYKGFQVAPAELEGLLVSHPEILDAVVIPFPDAEAGEVPVAYVVRSPNSSLTENDVKKFIAGQVASFKRLRKVTFINSVPKSASGKILRRELIQKVRSNM

SEQ ID NO: 28 - protein, 757 aa, Cannabis sativa AAE12
MYMYQEVYLVPTLSYLYLVVVLLPSIFFSFRRMAFKSLDSVTSSDIAALGIEPQLAHSLHGRLAEIVSNHGSATPHTWRCISSHLLSPDLPFSLHQMLYYGCYKDFGPDPPAWIPDAENAISTNVGKLLEKRGKEFLGVKYKDPISNFSDFQEFSVTNPEVYWRTILDEMNISFSKPPECILRENFSRDGQILNPGGEWLPGAFINPAKNCLDLNCKSLDDTMILWRDEGKDDLPVNKMTLKELRSEVWLVAYALKELELEGGSAIAIDMPMNVHSVVIYLAIVLAGYVVVSIADSFAAPEISTRLKISKAKAIFTQDLIVRGEKTIPLYSRIVEAQSPLAIVIPSKGFSVSAQLRHGDVSWHDFLNRANKFKNYEFAAVEQPIDAYTNILFSSGTTGEPKAIPWTQATPFKAAADAWCHMDIQKGDVVAWPTNLGWMMGPWLVYASLLNGASIALYNGSPLGSGFAKFVQDAKVTMLGVIPSIVRTWKSTNCVAGYDWSTIRCFSSTGEASNIDEYLWLMGRAYYKPVIEYCGGTEIGGGFVTGSLLQAQSLAAFSTPAMGCSLFILGSDGYPIPKHKPGIGELALGPLMFGASKTLLNADHYDVYFKRMPSLNGKVLRRHGDMFELTSKGYYHAHGRADDTMNLGGIKVSSVEIERICNEADEKVLETAAIGVPPLAGGPEQLVIAVVLKNSDRTTVDLNQLRLSFNSAVQKKLNPLFRVSRVVPLSSLPRTATNKVMRRILRQQFTQLDKSSKI

SEQ ID NO: 29 - protein, 726 aa, Ziziphus jujube
MAHKSLDGITASDIEALGIEPEVAKSLHGRLTKIIRNYGTATPDTWSNISRHILSPDLPFSFHQMMYYGCYKDFGPDPPAWIPDLEAAVSTNVGQLLERQGKEFLGSRYKDPISSFSDFQEFSVKNPEVYWKTILDEMNVSFSIPPQCILRENVSGERHFSHPGGEWLPGAFVNPANNCLSLNYKRNLDDSMVLWRDEGKDDLPINKMTLKELREEVWLVAHALEKLGLDKGSAIAIDMPMDVRSVIIYLAIVLAGYVVVSIADSFAPLEISTRLRISQAKAIFTQDLIIRGEKCIPLYSRIVEAESPMAIVIPTRGSSFSIKLRDGDVAWNDFLERVGDFKKIEFAAVDQPIEAFTNILFSSGTTGEPKAIPWTHATPFKAAADAWCHMDIQKGDVVCWPTNLGWMMGPWLVYASLLNGASIALYNGSPLGSGFAKFVQDAKVTMLGVIPSIVRTWKSSNCVAGYDWSTIRCFGSTGEASNVDEYLWLMGRACYKPVIEYCGGTEIGGGFVSGSLLQAQSLAAFSTPAMGCSLYILGSNGLPIPQNQPGIGELALDPLMFGASRTLLNADHYDVYFKGMPVWNGKVLRRHGDMFELTSRGYYHAHGRADDTMNIGGIKVSSVEIERICNEVDDSVLETAAIGVPPLGGGPEQLVIAVVFKDSNNPKEDLNQLRISFNSAVQKKLNPLFRVSRVVPLLSLPRTATNKVMRRILREQFSQHDQSSKI

SEQ ID NO: 30 - protein, 727 aa, Trema orientale
MGYKSLDSVTASDIAALGIDPELAETLHGRLADVIRNYASATPPDTWRYVSANILSPHLPFSFHQMMYYGCYQDFGPDPPAWIPDLENAISTNVGKLLERRGKEFLGSSYKDPISNFSDFQEFSVTNPEVYWKTILDEMNVSFSKPPQCILLENFPGDGKLLHPGGEWLPGAYVNPAKNCLSLNSKRSLDDTMIIWRDEGKDDLPVNKMTLEELRSEVWLVAYALKELGLEGGSAIAIDMPMNVHSVVIYLAIVLAGYVVVSIADSFAAREISTRLKISNAKAIFTQDLIIRGEKSIPLYSRIVEAQSPTAIVIPTRGSSFSAKLRQDDISWHDFLERAKAFKKREFAAIEQPVDAYTNILFSSGTTGEPKAIPWTHATPFKAAADAWCHMDIQKGDVVAWPTNLGWMMGPWLVYASLLNGASIALYNGSPLGSGFAKFVQDAKVTMLGVIPSIVRTWKSTNSIASYDWSTIRCFSSTGEASNVDEYLWLMGRACYKPVIEYCGGTEIGGGFVTGSLLQAQSLAAFSTPAMGCSLFVLGSDGYPIPKNKPGIGELALGPLMLGASKTLLNADHYDVYFKGMPSWNGKVLRRHGDMFEFTSRGYYRAHGRADDTMNLGGIKVSSVEIERICNEADDEVLETAAIGVPPPTGGPEKLVIAVVFKNPENTGADLNQLRLSFNSAVQKKLNPLFRVSHVVPLPSLPRTATNKVMRRILRQQLAQLDQSSKI

SEQ ID NO: 31 - protein, 727 aa, Parasponia andersonii
MGYKSLDSVTASDIAALGIDPELAETLHGRLADVIRNYASATPPDTWRYVSANILSPHLPFSFHQMMYYGCYQDFGPDPPAWIPDLENAISTNVGKLLERRGKEFLGSSYKDPISNFSDFQEFSVTNPEVYWKTILDEMNISFSKPPQCILRENFPGDGQLLHPGGEWLPGAYVNPAKNCLSLNSKRSLDDTMIIWRDEGKDDLPVNKMTLEEFRSEVWLVAYALKELGLERGSAIAIDMPMNVHSVVIYLAIVLAGYVVVSIADSFAAREISTRLKISKAKAIFTQDLIIRGEKSIPLYSRIVEAQSPTAIVIPTRGFSFSAKLRQGDISWHDFLERAKAFEKREFAASEQPVDAYTNILFSSGTTGEPKAIPWTQATPFKAAADAWCHMDIQKGDVVAWPTNLGWMMGPWLVYASLLNGASIALYNGSPLGSGFAKFVQDAKVTMLGVIPSIVRTWKSTNSVAFYDWSTIRCFSSTGEASNVDEYLWLMGRACYKPVIEYCGGTEIGGGFVTGSLLQAQSLAAFSTPAMGCSLFILGSDGYPIPKNKPGIGELALGPLMLGASKTLLNFDHYDVYFKGMPWWNGKVLRRHGDMFEFTSSGYYRAHGRADDTMNLGGIKVSSVEIERICNEADDEVLETAAIGVPPPTGGPEKLVIAVVFKNPENTGADLNPLRLSFNSAVQRKLNPLFRVSHVVPLPSLPRTATNKVMRRILRQQLAQLDQSSKI

SEQ ID NO: 32 - protein, 726 aa, Prunus avium
MAYKSLDHVTVSDIEALGIESEAAKRLHASLTNIIQNYGPATPDTWRNITAHVLSPELPFSFHQMLYYGCYKDFGPDPPAWLPDSETTNLTNVGQLLERRGKEFLGSRYKDPMSSFSDFQEFSVSNPEVYWKAVLDEMNASFSIPPQCILRENLSGDGQLSVLGGQWLPGAFGNPAKNCLSLNRKRSLNDTMVIWRDEGNDDLPLNKMTLKELRTEVWLVAHALKALGLEKGSAIAIDMPMHVNSVIIYLAIVLAGYVVVSIADSFAPPEISTRLKISEAKAIFTQDLIVRGEKSLPLYSKIVAAQSPMAIVILTKGSNSSMKLRDGDISWHDFLETVKDFKEDEFAAVEQPIEAFTNILFSSGTTGEPKAIPWTHATPFKAAADAWCHMDIQIGDVVSWPTNLGWMMGPWLVYASLLNGASIALYNGSPLGSGFPKFVQDAKVTMLGVIPSIVRTWKSTNSVSGYDWSTIRCFGSTGEASNVDEYLWLMGRARYKPIIEYCGGTEIGGGFVSGSLLQAQSLAAFSTPAMGCSLFILGNDGVPIPQNEPGVGELALGPLIFGASSTLLNADHYDVYFKGMPFWNGKVLRRHGDVFERTSRGYYHAHGRADDTMNLGGIKVSSVEIERICNEVDSEVLETAAIGVPPAVGGPEQLVLAVVFKNSDNQTADLNQLRTSFNSAVQKKLNPLFKVSRVVPLPSLPRTATNKVMRRILREQFAQLDQSAKL

SEQ ID NO: 33 - protein, 727 aa, Morus notabilis
MTDKSLDGVTASNIAALGIAPDVADGLHGRIAEVVRIYGPANPDTWRQISTRVLSPDLPFAFHQMLYHSCFNGFGPDPPAWIPDPEAAILTNVGKLLERRGKEFLGSRYKDPISNFSDFQEFSVTNPEVYWRTIFNEMNVSFSNPPECIFHENVPGGGQVSHPGGQWLPGAYVNPAMNCLSVNSKRSLDDASIVWRDEGKDDLPVNTMTLEELRSEVWLVAHALKELGLERGSAIAIDMPMHVHSVVIYLAIVLAGYVVVSIADSFAAGEISTRLKISKAKAIFTQDLIIRGEKSIPLYRRVVEAQSPMAIVIPTRGSSFSTQLRHGDIGWHDFLERVKEFKKCEFTAAEQPVDAFTNILFSSGTTGDPKAIPWTQATPFKAAADAWCHMDIQKGDVVAWPTNLGWMMGPWLVYASLLNGASIALYNGSPLGSSFAKFIQDAKVTMLGVIPSIVRTWKSMNSVSGYDWSTIRCFGSTGEASNVDEYLWLMGRACYKPVIEYCGGTEIGGGFVTGSLLQAQALAAFSTPAMGCSLFILGSDGYPIPKNKPGIGELALGPVMFGSSMTLLNADHYDVYFKGMPLWNGKVLRRHGDMFEITSRGYYRAHGRADDTMNLGGIKVSSVEIERLCNEVDNSILETAAIGVPPPAGGPEQLVIAVVFKDPDSNITTDLNQLRMSLNSAVQKKLNPLFRVSRVVPLQSLPRTATNKVMRRILRQQFVQLDQTSKM

SEQ ID NO: 34 - protein, 725 aa, Rosa chinensis
MSYKSLDAVTVADIAALGIEPELANRLHGSLAKIIADHGAATPDTWRSITGHVLSPDLPFSFHQMMYYGCYKDFGPDPPAWLPDPETAVLTNAGQLLERRGKEFLGSQYKDPISSFSDFQEFSVSNPEVYWKTVLDEMNVSFYKPPQCILRENLSGDGHLLVPGVQWLPGACVNPAKNCLSLNSKRSLNDTMVVWRDEGKDDLPLNKMTLKELRAEVWLVAHALQAQGLEKGSAIAIDMPMNVISVVIYLAIVLAGYVVVSIADSFAPPEISTRLKISEAKAIFTQDVIVRGEKSLPLYSKIVDAQSPMAIVLLTRGSKSSVKLRDGDISWHDFLNTVKDFKDEFAAVEQPVEAFTNILFSSGTTGDPKAIPWTHSTPFKAAADAWCHMDIRKGDVIAWPTNLGWMMGPWLVYASLLNVASIALYNGSPLGPGFSKFVQDAKVTMLGVIPSIVRTWKSTNSTSGYDWSAIRCFSSTGEASNVDEYLWLMGRAGYKPIIEYCGGTEIGGAFVSGSLLQAQSLASFSTPAMGCSLFILGTDGSPIPQNEPGVGELALGPLMFGASSTLLNADHYEVYFKGMPLWNGKVLRRHGDLFERTSRGYYHAHGRADDTMNLGGIKVSSVEIERICNAIDTNILETAAIGVPPAGGGPEQLVIAVVFKNSDNPPADLNQLRASFNSAVQKKLNPLFKVSRVVPLPSLPRTATNKVMRRILRQQFAQVDQGAKL

SEQ ID NO: 35 - protein, 729 aa, Citrus sinensis
MATYNYKALDCITSCDIEALGIPSKLAEQLHEKLAEIVNTHGAATPATWQNITTHILSPDLPFSFHQLLYYGCYKDFGPDPPAWIPDPEAAKVTNVGKLLQTRGEEFLGSGYKDPISSFSNFQEFSVSNPEVYWKTVLNEMSTSFSVPPQCILRENPNGENHLSNPGGQWLPGAFVNPAKNCLSVNSKRSLDDIVIRWRDEGDSGLPVKSMTLKELRAEVWLVAYALNALGLDKGSAIAIDMPMNVNSVVIYLAIVLAGYIVVSIADSFASLEISTRLRISKAKAIFTQDLIIRGDKSIPLYSRVIDAQAPLAIVIPAKGSSFSMKLRDGDISWFDFLERVRKLKENEFAAVEQPVEAFTNILFSSGTTGEPKAIPWTNATPFKAAADAWCHMDIRKADIVAWPTNLGWMMGPWLVYASLLNGASIALYNGSPLGSGFAKFVQDAKVTMLGVVPSIVRTWKSTNCIDGYDWSSIRCFGSTGEASNVDEYLWLMGRALYKPVIEYCGGTEIGGGFITGSLLQAQSLAAFSTPAMGCKLFILGNDGCPIPQNVPGMGELALSPLIFGASSTLLNANHYDVYFSGMPSRNGQILRRHGDVFERTSGGYYRAHGRADDTMNLGGIKVSSVEIERICNAVDSNVLETAAIGVPPPDGGPEQLTIVVVFKDSNYTPPDLNQLRMSFNSAVQKKLNPLFKVSHVVPLPSLPRTATNKVMRRVLRKQLAQLDQNSKL

SEQ ID NO: 36 - protein, 729 aa, Citrus clementina
MATCNYKALDCITSYDIEALGIPSKLAEQLHEKLAEIVNTHGAATPATWQNITTHILSPDLPFSFHQLLYYGCYKDFGPDPPAWIPDPEAAKVTNVGKLLETRGEEFLGSGYKDPISSFSNFQEFSVSNPEVYWKTVLNEMSTSFSVPPQCILRENPNGENHLSNPGGQWLPGAFVNPAKNCLSVNSKRSLDDIVIRWCDEGDGGLPVKSMTLKELRAEVWLVAYALNALGLDKGSAIAIDMPMNVNSVVIYLAIVLAGYIVVSIADSFASLEISARLRISKAKAIFTQDLIIRGDKSIPLYSRVIDAQAPLAIVIPAKGSSFSMKLRDGDISWLDFLERVRKLKENEFAAVEQPVEAFTNILFSSGTTGEPKAIPWTNATPFKAAADAWCHMDIRKADIVAWPTNLGWMMGPWLVYASLLNGASVALYNGSPLGSGFAKFVQDAKVTMLGVVPSIVRTWKSTNCIDGYDWSSIRCFGSTGEASNVDEYLWLMGRALYKPVIEYCGGTEIGGGFITGSLLQAQSLAAFSTPAMGCKLFILGNDGCPIPQNVPGMGELALSPLIFGASSTLLNANHYDVYFSGMPSWNGQILRRHGDVFERTSGGYYRAHGRADDTMNLGGIKVSSVEIERICNAVDSNVLETAAIGVPPPDGGPEHLTIVVVFKDSNYRPPDLNQLRMSFNSAVQKKLNPLFKVSHVVPLPSLPRTATNKVMRRVLRKQLAQLDQNSKL

SEQ ID NO: 37 - protein, 726 aa, Arachis duranensis
MAYKSLTSITVSDIESVGISTEVASAFHRRLKEIIATHGAGTPATWHNITNTILTPDLPFSFHQMLYYACYIDFGPDPPAWIPDPECALSTNVGQLLERRGKEFLGSAYKDPISSFSDFQKFSVSNPEVFWKNVLDEMNISFSTPPECILRENLPGESSLTHPGGQWLPGASINPAKNCLVENAKRSLNDTAIIWRDEHHDDLPVQRMTFKELQEEVWLVAYALEALGLEKGSAIAIDMPMHVKSVVIYLAIVLAGYVVVSIADSFAAGEISTRLNISNAKVIFTQDLIIRGDKSIPLYSRVVEAKSPLAVVIPTRGSEFSMELRNGDFSWHDFLDRANSLKGKEFVAVEQPVEAFTNILFSSGTTGEPKAIPWTNITPLKAAADAWCHLDIRKGDVVSWPTNLGWMMGPWLVYASLINGASMALYNGSPLGSGFAKFVQDAKVTMLGVIPSIVRSWKSANSTSGYDWSAIRCFGSTGEASNVDEYLWLMGRALYKPVIEYCGGTEIGGGFITGSLLQPQSVAAFSTPAMCCSLFILDEEGHPIPQDVPGMGELALGPIMFGASITLLNADHYAVYFKGMPVYNGKVLRRHGDVFERTAKGYYHAHGRADDTMNLGGIKVSSVEIERLCNGVDSSILETAAIGVPPSGGGPEQLVVAVVFKNPSTTTQDLHQLRISFNSALQKKLNPLFRVSRVVSLPSLPRTASNKVMRRVLRQQLSENNQSSKI

SEQ ID NO: 38 - protein, 729 aa, Quercus suber
MGYKALDRITRSDIEEEVGIAAAAGVAERIHERLTEIVRNYGADTPDTWRSICERVLSPDLPFSLHQMMFYGCYNGYGTDPPAWIPDPKTAILTNVGQLLERRGKEFLGSKYKDPISSFSDLQEFSVSNPEVYWKTVLDEMSISFSVPPQCILRDSPFGESHSSYPGGQWLPGAFLNPAENCLSLNSKRSLEDIAVIWRDEGDDILPVNRMTVREFRAEVWLVAHAIKTLGLDKGSAIAIDMPMNVNSVVIYLAIVLAGYVVVSIADSFAPREISTRLKISEAKAIFTQDLIIRGDKSIPLYSRIVEAQSPMAVVIPARGSSFSMKLRDGDISWHDFLGRVKNFKECEFAAVEQPVEAFTNILFSSGTTGEPKAIPWTSATPLKAAADAWCHLDIQKGDVVAWPTNLGWMMGPWLVYASLLNGASMALYNGSPLSSGFAKFVQDAKVTMLGVIPSIVRAWKSTNCMAGYDWSAIRCFGSTGEASNVDEYLWLMGRACYKPIIEYCGGTEIGGGFITGSFLQAQSLAAFSTPAMGCSLFILGSDGYPIPENVPGIGELALGPLMFGASNKLLNADHHDVYFKGMPLWKGRVLRRHGDVFERTSRGYYHAHGRADDTMNLGGIKVSSVEIERICNAADNSVLETAAIGVPPSGGGPEQLVIAVVFKESENMTADLNQLRISFNSAVQKKLNPLFRVSQVVPLSSLPRTASNKVMRRVLRQQLTQGDRNPKL

SEQ ID NO: 39 - protein, 726 aa, Theobroma cacao
MVYKSLDSVTVKDIEASGISSQLAEEIHRKVTEIVDGYGAATPESWNRISKHVLTPNLPFSLHQMMYYGCYKDFGPDPPAWMPDPESALLTYVGLLLEKHGKEFLGSKYKDPISSFSHLQEFSVSNPEVYWKTVLDEMCVNFSVPPDCILHESTSEESRILNPGGKWLPGAFVNPAKNCLIVNSKRGLDDIVIRWRDEGDDDLPVKSMTLKELQLEVWLVAHALNALGLERGSAIAIDMPMNVYSVIIYLAIVLAGYIVVSIADSFAPLEISTRLKISEAKAIFTQDLIIRGEKSIPLYSRVVEAEAPMAIVIPARGFSCSAKLRDGDISWSDFLERVRELKGDVFEAVEQPVEAFTNVLFSSGTTGEPKAIPWTHVTPLKAAADAWCHMDIHSGDIVAWPTNLGWMMGPWLVYASLLNGASMALYNGSPLSSGLAKFVQDAKVTMLGVIPSIVRAWKSTNCVAGYDWSSIRCFSSTGEASNVDEYLWLMGRACYKPIIEYCGGTEIGGGFVSGSFLQPQSLAAFSTPAMGCRLFILGDDGHPIPQDAPGMGELALGPLMFGSSSTLLNASHYDVYFKEMPSWNGLILRRHGDVFERTSRGYYHAHGRADDTMNIGGIKVSSVEIERICNAVDSSVLETAAIGVPPADGGPERLVIAVVFKDPDNATPDLNQLRKSFNSAVQKNLNPLFRVSHVVALSALPRTASNKVMRRVLRKQLAQVDQNSKL

SEQ ID NO: 40 - protein, 726 aa, Jatropha curcas
MAHNALGAISVSDIEALGISSELAEKLYTHVSQIINNYGSATPETWSRISKHVLTPDLPFSFHQMMFYGCYKDFGPDPPAWLPDPKSAALTNVGQLLQRRGKEFLGEGYVDPISSFSAFQEFSVSNPEVYWKTVLDEMDVAFSVPPQCILREDLSGESSFLNPGGQWLPGAYVNPAKNCLSLNSKRILDDTVIRWRCEGSDDLPVSSMTLEELRTEVWLVAYALNSLGLDRGSAIAIDMPMNVKAVVIYLAIVLAGYVVVSIADSFAPLEISTRLKISKAKAIFTQDLIIRGDKNIPLYSRVVDAQSPMAIVIPTKGSSFSMKLRDGDISWHDFLEKVQNLRGNEFAAVEQPIEAFTNILFSSGTTGEPKAIPWTSATPFKAAADAWCHMDIRKGDIVAWPTNLGWMMGPWLVYASLLNGACIALYNGSPLGSSFAKFVQDAKVTMLGVIPSIVRTWKTANTTAGYDWSAIRCFGSTGEASNVDEHLWLMGRALYKPIIEYCGGTEIGGGFVSGSFLQPQSLAAFSTPAMGCSLFILGDDGHPIPHDVPGIGELALGPLMFGASSSLLNADHYNVYYKGMPVWNGKILRRHGDVFERTSRGYYHAHGRADDTMNLGGIKVSSVEIERICNVVDSSILETAAIGVPPPQGGPEQLVIAVVFKNLENSTTDLEQLRKSFNSAVQKKLNPLFRVSRVVPHPSLPRTASNKVMRRILRQQFVQQEQNSKL

SEQ ID NO: 41 - protein, 729 aa, Populus trichocarpa
MASLHYKALDSISVSDIEALGISSSIALQLYEDISEIINTHGPSSPQTWTLLSKRLLHPLLPFSFHQMMYYGCFKDFGPDPPAWSPDPEAAMLTNVGQLLERRGKEFLGSAYKDPISSFSNFQEFSVSNPEVYWKTILDEMSISFSVPPQCILSENTSRESSLANPGGQWLPGAYVNPAKTCLTLNCKRNLDDVVIRWRDEGNDDMPVSSLTLEELRSEVWLVAYALNALGLDRGSAIAIDMPMNVESVIIYLAIVLAGHVVVSIADSFAPLEISTRLKISEAKAIFTQDLIIRGDKSIPLYSRVVHAQAPMAIVLPTKGCSFSMNLRDGDISWHDFLEKATDLRGDEFAAVEQPVEAFTNILFSSGTTGEPKAIPWTHLTPFKAAADAWCHMDIRKGDIVAWPTNLGWMMGPWLVYASLLNGASIALYNGSPLGSGFAKFVQDASVTMLGVIPSIVRIWKSANSTSGYDWSAIRCFASTGEASSVDEYLWLMGRAQYKPIIEYCGGTEIGGGFVSGSLLQPQSLAAFSTPAMGCSLFILGDDGHPIPQNVPGMGELALGPLMFGASSTLLNADHYNVYFKGMPLWNGKILRRHGDVFERTSRGYYHAHGRADDTMNLGGIKVSSVEIERVCNAVDSNVLETAAVGVPPPQGGPEQLVIAVVFKDSDESTVDLDKLRISYNSAVQKKLNPLFRISHVVPFSSLPRTATNKVMRRVLRQQLSQQDQNSKL

SEQ ID NO: 42 - protein, 721 aa, Hevea brasiliensis
MSSYKALDAISVSDIEALGISSKLADKLYKDVADIIANYGASTPQTWTHISKHVLNPDLPFSLHRMMFYACYKDFGSDPAAWSPDPKTAALTNVGQLLERRGKEFLGSLYVDPISSFSAFQEFSVSNPEVYWKTVLDEMSISFSVPPQCILLENPESPGGQWLPGAYVNPARNCLSLNRERTLDDTVITWRDEGSDDLPLSSMTLGELRTEVWLVAYALNTLGLDRGSAIAIDMPMNVKSVVIYLAIVLAGYAVVSIADSFASPEMSTRLKISEAKAIFTQDLIIRGDKSIPLYSRVVDAQSPMAIVIPTKGSSFSMKLRGGDISWHDFLERVENIRGDEFAAVEQPIEAFTNILFSSGTTGDPKAIPWTNATPFKAAADAWCHMDIRRGDVVAWPTNLGWMMGPWLVYASLLNGACIALYNGSPLGSGFAKFVQDAKVTMLGVIPSIVRTWKSANSTAGYDWSAIRCFGSTGEASNVDEYLWLMGRAHYKPIIEYCGGTEIGGGFVSGSLLQPQSLAAFSTPAMGCSLFILGDDGHPFPQNVPVMGELALGPLMFGASSSLLNANHYNVYYKGMPVWNGKILRRHGDVFEHTSRGYYRAHGRADDTMNLGGIKVSSVEIERICNAVDSSILETAAIGVPPPQGGPERLVIAVVFNDPDNSTTDLEQLRKSFNSAVQKKLNPLFRVSHVVALPSLPRTATNKVMRRILRQQFVQQEQNSKL

SEQ ID NO: 43 - protein, 726 aa, Vitis vinifera
MAGKTLDSITSQDIAALGIPSEEAEKLHQTLLQIITSCGAATPQTWSRISKELLNPDLPYSLHQMMYYGCYSHFGPDPPAWLPDPENVMLTNVGQLLERRGKEFLGSRYKDPISSFSDFQKFSVSNPEVYWKTVLDELSISFSVPPQCVLYDNPSRENGLSYPGGQWLPGAFINPARNCLSVNDKRTLDDTVVIWHDEGDDGMPINRMTLEELRREVWSVAYALDTLGLEKGSAIAIDMPMNASSVVIYLAIVLAGYIVVSIADSFASREISTRLKISNAKAIFTQDFIIRGDKSLPLYSRVVDAQSPTAIVIPAGGSSFSMKLRDGDMSWHDFLQRAINSRDDEFAAIEQPIEAFMNILFSSGTTGEPKAIPWTNATPLKAAADAWCHMDIRKGDIVAWPTNLGWMMGPWLVYASLLNGATIALYNGAPLGSGFAKFVQDAKVTMLGVIPSIVRTWKSTNCTAGLDWSSIRCFASTGEASSVDEYLWLMGRAQYKPIIEYCGGTEIGGGFVTGSLLQAQSLASFSTPAMGCSLFIIGDDGNLLPQDASGMGELALGPLMFGASTTLLNADHYDVYFKGMPIWNGKVLRRHGDVFERTSRGYYRAHGRADDTMNIGGIKVSSVEIERICNTVHSSVLETAAIGMPPPAGGPERLMIVVVFKDSNNSIPDLNELRIAFNSEVQKKLNPLFRVSHTVPVPSLPRTATNKVMRRVLRQQLAQLSSTSKF

SEQ ID NO: 44 - protein, 722 aa, Manihot esculenta
MDNKVLDAISVSDIEALGISSPLAHKLCKDVADIVANYGAATPQTWTHISKHVLHPDLPFSFHQMMFNACYKDFGTDPPAWSPDLKSAALTNVGHLLERRGKEFLGSLYVDPISSFSAFQEFSVSNPELYWKTVLDEMNISFSVPAQCILLENSYGESPGGQWLPGAYVNPAKNCLSLNCKRTLDDTVIRWRDEGSDELPLSSMTLDELRTEVWLVAYALNRLGLDRGSAIAIDMPMNVKSVVIYLAIVLAGYVVVSIADSFAPLEIATRLKISEAKAIFTQDLIIRGDKSIPLYSRVVDAQSPMAVVIPAKGSSFSMKLRDGDISWHDFLERVENRRGDEFAAVEQPIEAFTNILFSSGTTGEPKAIPWTNATPFKAAADAWCHMDIHKGDVVAWPTNLGWMMGPWLVYASLLNGACIALYNGSPLGSGFAKFVQDAEVTMLGVIPSIVRTWKSANSTAGYDWSSIRCFGSTGEASNIDEYLWLMGRAHYKPVIEYCGGTEIGGGFVSGSLLQPQSLAAFSTPAMGCSLFILGDDGHPIPHNAPGMGELALGPLMFGASSSLLNADHYNVYFKGMPVWNGKILRRHGDVFERTSRGYYHAHGRADDTMNLGGIKVSSVEIERICNAVDNSILETAAIGVPPSQGGPERLVIAVVFKNPDNTTRDLEQLRKTFNSAVQKKLNPLFRVSHVVALPTLPRTATNKVMRRILRQQFVQQEQTAKL

SEQ ID NO: 45 - protein, 722 aa, Nicotiana attenuate
MAHQNYKGLDSVTVADVEALGIASELAGEIHEKLTRIVRNYSATTPQTWHHISKEILTPKLPFSLHQMMYYGCYKDFGPDPPAWLPDSKNVGLTNIGQLLERRGKEFLGSNYEDPISSFSDFQRFSVSEPEVYWKTILEEMNVSFSVPPECILRESPSHPGGQWLPGARVNPAKNCLSFRKRTLSDVAIVWRSEGNDEAPVEKMTLKELCESVWAVAYALETLGLEKGSAIAIDMPMDVNSVVIYLAIVLAGYVVVSIADSFAPSEISTRLILSKAKAIFTQDFIFRGDKKIPLYSRVVDARSPTAIVIPNRASSLSIQLRDGDISWPEFLERVKDSRGLEFVAVEQPITAFTNILFSSGTTGEPKAIPWSLLSPFKSAADGWCHMDIKKGDVVAWPTNLGWMMGPWLVYASLLNGASIALYNGSPLDSGFAKFVQDAKVTMLGVIPSIVRTWKAKNSPDGFDWSTIRCFGSTGEASSVDEYLWLMGRAEYKPIIEYCGGTEIGGSFVSGSLLQPQSLAAFSTAVMGCSLHILGEDGLPIPSDVPGTGELALGPLMFGASSTLLNADHNEIYFKGMPVLNGKVLRRHGDVFERTSKGYYHAHGRADDTMNLGGIKVSSLEIERICNAADENILETAAVGVPPAGGGPEKLVIAVVFKDSANLEHNMDKLMISFNTALQRKLNPLFKVSSIVPLPLLPRTATNKVMRRVLRQQFSQAEQGSKL

SEQ ID NO: 46 - protein, 722 aa, Solanum pennellii
MANQNYRTLDSVTVADVEALGIPTELAEKLHEELTRIVRNYGSVTPQTWHHISKELLTPNLPFSFHQMMYYGCYKDFGSDPPAWLPDPKTARLTNIGQLLERRGMEFLGSKYDDPISSFSDFQRFSVSDQEVFWKTILEEMNISFSVPPECILRESPSHPGGQWLPGSRANPAKNCLSLRKRTLSDVAIIWRSEGNDEAPVEKMTCQELRESVWEVAYALESLGLEKGSAIAIDMPMDVNSVVIYLAIVLAGYVVVSIADSFAPSEISTRLILSKAKAIFTQDFIPRGEKKIPLYSRVVEAHSPMAIVIPNRVSSLSIELRDGDISWPDFLDRVKDSKGLEFVAVEQPIDAFTNILFSSGTTGDPKAIPWTLLTPFKAAADGWCHMDIKNGDVVAWPTNLGWMMGPWLVYAALLNGASIALYNGSPLGSGFAKFVQDAKVTMLGVIPSIVRTWKAKNSPDGYDWSTIRCFGSTGEASSVDEYLWLMGRAEYKPIMEYCGGTEIGGSFVSGSMLQPQSLAAFSTAVMGCSLHILGDDGFPIPSDVPGIGELALGPLMFGASSTLLNADHNEIYFKGMPVLNGKVLRRHGDVFERTSKGYYHAHGRADDTMNLGGIKVSSLEIERICNVVDENILETAAVGVPPAAGGPEKLVIAVVFKDSDNLEQKLVNLLISFNTALQRKLNPLFKVSSIVPLPSLPRTATNKVMRRVLRQQFSQADQGSRL

SEQ ID NO: 47 - protein, 744 aa, Nelumbo nucifera
MAIKSLDCVTVEDITGLGISSDAAKKLHGDLTEILRENANSAADTWKKISKRILNPNLPFAFHQMMYYGCFKDFGSDPPAWIPDQETAILTNVGRFLEKRGKEFLGSKYKDPITSFLDFQEFSVSNPEVYWKMVLDEMNISFSVPPSCILYEHTSEGGHLSYPGGQWLPGAILNCAENCLNLNGKRSLNDTMIIWRDEGDDNLPVKHMMLKQLRSEVWLVAYALDTLGLAKGSAIAIDMPMNVTAVVIYLAIVLAGYIVVSIADSFAPLEISTRLKISNAKAIFTQDVIIRGDKILPLYSRVVDAQAPLAIVVPSRGSSLKMELRGCDMSWHAFLERVEHFKKDEFAAVQQPVDAFTNILFSSGTTGEPKAIPWTHATPLKAAADAWCHMDIQKGDVVAWPTNLGWMMGPWLVYASLLNGASMALYNGSPLGSGFAKFVQDAKVTMLGVVPSIVRAWKNTNCTAGFDWSSIRCFSSTGEASNVDEYLWLMGRAHYKPVIEYCGGTEIGGGFVSGSLLQAQSLAAFSTPAMGCTLFILCSDGNPILQNTPGIGELALAPIMLGASNTLLNANHYDVYFRGMPMWNGKVLRRHGDEFECTSKGYYRAHGRADDTMNLGGIKVSSIEIERICNGVDDTILETAAIGVPPVGGGPEKLAIAVVFKDSNSLPDVDQLKMKFNSSLQKKLNPLFRVSAVVPVSSLPRTASNKVMRRVLRQQFSQLYQASTSRIASGFLLQSPPQRPSTSL

SEQ ID NO: 48 - protein, 725 aa, Momordica charantia
MDYKTLDSITVIDIEALGVASEVAEKLHGLLSEIIRSHGNGTPETWRHISKRVLSPDLPFSFHQMMYYGCYKHYGPDPPAWIPEPENAVFTNVGQLLKRRGKEFLGSNYRDPLSSFSSFQEFSVSNPEVYWRTMLDEMHITFSKPPHCILQMNDSTESQFSSPGGQWLPGAVFNPAKDCLSLNENRSLDDVAIIWRDEGCDNLPVKRLTLGELRTDVWLIAHALNSIGFEKGTAIAIDMPMNVNAVVIYLGIVLAGHVVVSIADSFSAREISTRLDISKAKAIFTQDLIIRGDKSIPLYSRVVDAQSPMAIVIPSRSTGFSRKLRDEDISWHAFLERVEDLRGVEFAAVEQAAESFTNILFSSGTTGEPKAIPWTLVTPLKAAADAWCYMDIHKGDVVAWPTNLGWMMGPWLVYASLLNSASMALYNGSPLGSGFVKFVQDAKVTMLGVIPSIVRSWKSTNCTSGYDWSSIRCFASTGEASNVDENLWLMGRACYKPVIEICGGTEIGGGFITGSLLQPQALAAFSTPAMGCSLFILGNDGFPIPQNMPGIGELALGPFLFGASSTLLNADHYDIYFKGMPHWNGMVLRRHGDVFERSPRGYYRAHGRADDAMNLGGIKVSSVEIERICNTIDDSILETAAIGVPPLGGGPEQLVIAVVLKNPGETSPDLDKLKLCFNSSLQKNLNPLFRVHRVVPYPSLPRTATNKVMRRILRQQLAVERRTKL

SEQ ID NO: 49 - protein, 385 aa, Cannabis sativa
MNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKY

SEQ ID NO: 50 - protein, 399 aa, Humulus lupulus
MSSSITVDQIRKAQRAEGPATILAIGTATPANFIIQADYPDYYFRVTKSEHMTNLKKRFQRICDRTMIKKRHLVLSEDHLKENPNMCEFMAPSLDVRQDILVVEVPKLGKEACMKAIKEWDQPKSKITHFIFATTSGVDMPGADYQCAKLLGLSSSVKRVMMYQQGCFAGGTVLRIAKDIAENNKGARVLALCSEITTCMFHGPTESHLDSMVGQALFGDGASAVIVGAEPDESAGERPIYELVSAAQTILPNSEGAIDGHLMETRLTFHLLKDVPGLISNNIEKSLIEAFTPIGINDWNSIFWVTHPGGPAILDEVEAKLELKKEKLAISRHVLSEYGNMSSASVFFVMDELRKRSLEEGKSTTGDGLDWGVLFGFGPGLTVEMVVLHSVENKVKSET

SEQ ID NO: 51 - protein, 393 aa, Morus notabilis
MSMTPSVHEIRKAQRSEGPATVLSIGTATPTNFVSQADYPDYYFRITNSDHMTDLKDKFKRMCEKSMITKRHMYLTEEILKENPKMCEYMAPSLDARQDIVVVEVPKLGKEAAAKAIKEWGQPKSKITHLIFCTTSGVDMPGADYQLTKLLGLRPSVKRFMMYQQGCFAGGTVLRLAKDLAENNKGARVLVVCSEITAVTFRGPSHTHLDSLVGQALFGDGAAAVIVGADPDTSVERPIFELVSAAQTILPDSEGAIDGHLREVGLTFHLLKDVPGLISKNIEKSLVEAFTPIGISDWNSIFWIAHPGGPAILDQVETKLGLKQEKLSATRHVLSEYGNMSSACVLFILDEMRKKSVEEGKATTGEGLEWGVLFGFGPGLTVETVVLHSLPAV

SEQ ID NO: 52 - protein, 101 aa, Cannabis sativa
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRK

SEQ ID NO: 53 - protein, 104 aa, Cannabis sativa
MAVKHLIVLKFKDEITEAQKEEFFKTYVNLVNIIPAMKDVYWGKDVTQKKEEGYTHIVEVTFESVETIQDYIIHPAHVGFGDVYRSFWEKLLIFDYTPRKLKPK

SEQ ID NO: 54 - protein, 112 aa, Beauveria bassiana
MAPVTHIVLFEFKPDVTKAQRDEFSAEMLGLKDKCIHAKTQKPYILRSSGGTDNSIEGLQHGITHAFVVEFASVEDRQYYVKEDPAHIAFVNKLFPFLAKPYIIDFTPGEFN

SEQ ID NO: 55 - protein, 112 aa, Cordyceps brongniartii RCEF 3172
MAPVTHIVLFEFKPEVTKAQRDEFSAEMLGLKDKCIHSKTQKPYILRSSGGTDNSIEGLQHGITHAFVVEFASVEDRQYYVKEDPAHIAFVNKLFPSLAKPYIIDFTPGEFN

SEQ ID NO: 56 - protein, 112 aa, Cordyceps confragosa RCEF 1005
MAPITHVVLFEFKPEVDKAERDELSAEMLGLKDKCLHATTQKPYIIRSSGGTDNSIEGMQHGVTHAFVVEFASAEDRQYYVKEDPVHIAFVKKVFPRLAKPYIIDFTPGEFN

SEQ ID NO: 57 - protein, 112 aa, Cordyceps fumosorosea ARSEF 2679
MAPVTHIVMFEFKPEVTKAQRDEFSAEMLDLKNKCIHPKTNQAYILRSTGGTDNSIEGFQHGISHAFVVEFASPEDREYYVKEDPAHLAFVQKLFPSLAKPYVVDFTPGEFN

SEQ ID NO: 58 - protein, 112 aa, Cordyceps militaris CM01
MAPITHIVMFEFKSDVTKAQRDELSKEMLALKDNCIHAATQKPYIVHSHGGTDNSIEGFQHGISHVFVVEFASVEDRTYYVKEDPVHSRYVQKLLPFLVKPTVVDFTPGEFH

SEQ ID NO: 59 - protein, 116 aa, Torrubiella hemipterigena
MAPVIHIVMFQFKEDVSTETIKEMSDRMLGLKTNCIHATTKQPYILSSRGGTDMSIEGLTQGYTHAYVVEFASKEDRDYYVKEDPVHAAYVKDVVPLLIKPCIFDYHPGEFTHTKL

SEQ ID NO: 60 - protein, 395 aa, Cannabis sativa
MGLSSVCTFSFQTNYHTLLNPHNNNPKTSLLCYRHPKTPIKYSYNNFPSKHCSTKSFHLQNKCSESLSIAKNSIRAATTNQTEPPESDNHSVATKILNFGKACWKLQRPYTIIAFTSCACGLFGKELLHNTNLISWSLMFKAFFFLVAILCIASFTTTINQIYDLHIDRINKPDLPLASGEISVNTAWIMSIIVALFGLIITIKMKGGPLYIFGYCFGIFGGIVYSVPPFRWKQNPSTAFLLNFLAHIITNFTFYYASRAALGLPFELRPSFTFLLAFMKSMGSALALIKDASDVEGDTKFGISTLASKYGSRNLTLFCSGIVLLSYVAAILAGIIWPQAFNSNVMLLSHAILAFWLILQTRDFALTNYDPEAGRRFYEFMWKLYYAEYLVYVFI

SEQ ID NO: 61 - protein, 409 aa, Humulus lupulus
MELSSVSSFSLGTNPFISIPHNNNNLKVSSYCCKSKSRVINSTNSKHCSPNNNTSNKTTHLLGLYGQSRCLLKPLSFISCNDQRGNSIRASAQIEDRPPESGNLSALTNVKDFVSVCWEYVRPYTAKGVIICSSCLFGRELLENPNLFSWPLIFRALLGMLAILGSCFYTAGINQIFDMDIDRINKPDLPLVSGRISVESAWLLTLSPAIIGFILILKLNSGPLLTSLYCLAILSGTIYSVPPFRWKKNPITAFLCILMIHAGLNFSVYYASRAALGLAFAWSPSFSFITAFITFMTLTLASSKDLSDINGDRKFGVETFATKLGAKNITLLGTGLLLLNYVAAISTAIIWPKAFKSNIMLLSHAILAFSLIFQARELDRTNYTPEACKSFYEFIWILFSAEYVVYLFI

SEQ ID NO: 62 - protein, 352 aa, Saccharomyces cerevisiae
MASEKEIRRERFLNVFPKLVEELNASLLAYGMPKEACDWYAHSLNYNTPGGKLNRGLSVVDTYAILSNKTVEQLGQEEYEKVAILGWCIELLQAYFLVADDMMDKSITRRGQPCWYKVPEVGEIAINDAFMLEAAIYKLLKSHFRNEKYYIDITELFHEVTFQTELGQLMDLITAPEDKVDLSKFSLKKHSFIVTFETAYYSFYLPVALAMYVAGITDEKDLKQARDVLIPLGEYFQIQDDYLDCFGTPEQIGKIGTDIQDNKCSWVINKALELASAEQRKTLDENYGKKDSVAEAKCKKIFNDLKIEQLYHEYEESIAKDLKAKISQVDESRGFKADVLTAFLNKVYKRSK

SEQ ID NO: 63 - protein, 424 aa, Aspergillus terreus
MLPPSDSKDPRPWQILSQALGFPNYDQELWWQNTAETLNRVLEQCDYSVHLQYKYLAFYHKYILPSLGPFRRPGVEPEYISGLSHGGHPLEISVKIDKSKTICRLGLQAIGPLAGTARDPLNSFGDRELLKNLATLLPHVDLRLFDHFNAQVGLDRAQCAVATTKLIKESHNIVCTSLDLKDGEVIPKVYFSTIPKGLVTETPLFDLTFAAIEQMEVYHKDAPLRTALSSLKDFLRPRVPTDASITPPLTGLIGVDCIDPMLSRLKVYLATFRMDLSLIRDYWTLGGLLTDAGTMKGLEMVETLAKTLKLGDEACETLDAERLPFGINYAMKPGTAELAPPQIYFPLLGINDGFIADALVEFFQYMGWEDQANRYKDELKAKFPNVDISQTKNVHRWLGVAYSETKGPSMNIYYDVVAGNVARV

SEQ ID NO: 64 - protein, 391 aa, Streptomyces blastmyceticus
MESAGPGTGPQPPRTSGDFTPDTGVIAEMTGRPMRFDSDRYRPTDTYAEVACDKVCRAYEGLGADGGDRESLLAFLRDLTDPWGELPVGTPPEDACWVSIDGMPLETSVAWAGRKAGVRLSLESPRGPAKRRMEDGMALTRRLAGRPGVSVDPCLRVEDLFTDDDPQGYFTIAHAVAWTPGGHPRYKIFLNPAVRGREQAAARTEEAMIRLGLEQPWRALTEHLGGAYGPEHEPAALAMDLVPGDDFRVQVYLAHSGVSAEAIDAKSAVAADHVPGSFARALRGINGADDTPEWKRKPPVTAFSFGPGRAVPGATLYVPMIPVHGSDAAARDRVAAFLRSEGMDAVGYEAVLDAISDRSLPESHTQNFISYRGGDSPRFSVYLAPGVYREA

SEQ ID NO: 65 - protein, 374 aa, Marinactinospora thermotolerans
MAGDPFVDNGTVSSQRPLRAVPGRYPPGATHLDAAVDTLVRCHAALGRAPSEAEAAVCLLRRLWGRWGNTPVERPGWRSYVAVDGSPFELSAAWNGDGPAEVRVTVEATADPPTPEGNQEAGWEYLRGLSRHPGAATARVLALEDLFRPQTPHDRCWIMHGMASRPGADPLFKVYLDPDARGAAEAPSVLDEAMDRLGVRAAWQGLRGWLDEHGGSGRIGSLALDLADTDDARVKVYVQHAGLDWADIDRQAAVARGHVPGAFSAALEEITGTEVPPHKPPVTCFAFHRGVGVPTAATLYIPMPAGVPESDARRRSAAFMRRSGLDSAAYLAFLAAATGDGEGVRALQNFVAYRPAAPGGRPRFACYVAPGLYR

SEQ ID NO: 66 - protein, 439 aa, Pestalotiopsis fici W106-1
MAISTPSNGVSHVAKPLPNLKEVNKGIETDSEDRAFWWGALSEPLASLLEANHYTKEVQLHYLRWFYQWILPALGPRPLDGKPYYGSWITHDLSPFEYSLNWKEKSSKQTIRFTIEAVTKQSGTASDPINQLGAKEFLEAVSKDVPGMDLTRFNQFLEATNVPNDCVDDAIAKHPAHFPRSRVWIAFDLEHSGNLMAKSYFLPHWRAIQSGISANTIIGDTVKECNKADGSSYDGSLNAIESYLATFTRPEEAPQMGLLSNDCVAETPGSRLKVYFRSSADTLAKAKDMYNLGGRLKGPKMDASLKGISDFWYHLFGLDSSDPASDDKVCIGNHKCIFVYEMRSSQGSEPDIDVKFHIPMWQLGKTDGQISELLASWFESHGHPDLASRYKSDLGTAFPKHNITGKSVGTHTYISITHTPKTGLYMTMYLSPKLPEFYY

SEQ ID NO: 67 - protein, 316 aa, Streptomyces sp. CNZ306
MIGIDFLECLVSEGIEAEGLYSAIEESARMVDAPFSRDKVWPILSAFGGGFSDAGGVIFSLQAGKDVPEMEYSAQISAEVGDPYAHALATGVLNETDHPVSTVLAEIVSLAPTSEHYIDCGIVGGFKKIYANFPHDQQKVSRLADLPAMPRAVGANAEFFDRYGLDNVALIGVDYRNKTINLYFQAPAETAGNLDPKTVSAMLRETGMSTPSEEMVAYADRAYRIYATLGWDSPEVMRLAFAPQPRRSIDLAELPARLEPRIEQFMRATPHKYPGALINATAAKWSKKHEVLDLAAYYQVSALHLKAIQAEEGQSS

SEQ ID NO: 68 - protein, 300 aa, Streptomyces cinnamonensis
MMSGTADLAGVYAAVEESAGLLDVSCAREKVWPILAAFEDVLPTAVIAFRVATNARHEGEFDCRFTVPGSIDPYAVALDKGLTHRSGHPIETLVADVQKHCAVDSYGVDFGVVGGFKKIWVYFPGGRHESLAHLGEIPSMPPGLAATEGFFARYGLADKVDLIGVDYASKTMNVYFAASPEVVSAPTVLAMHREIGLPDPSEQMLDFCSRAFGVYTTLNWDSSKVERIAYSVKTEDPLELSARLGSKVEQFLKSVPYGIDTPKMVYAAVTAGGEEYYKLQSYYQWRTDSRLNLSYIGGRS

SEQ ID NO: 69 - protein, 307 aa, Streptomyces sp. KO-3988
MPGTDDVAVDVASVYSAIEKSAGLLDVTAAREVVWPVLTAFEDVLEQAVIAFRVATNARHEGDFDVRFTVPEEVDPYAVALSRSLIAKTDHPVGSLLSDIQQLCSVDTYGVDLGVKSGFKKVWVYFPAGEHETLARLTGLTSMPGSLAGNVDFFTRYGLADKVDVIGIDYRSRTMNVYFAAPSECFERETVLAMHRDIGLPSPSEQMFKFCENSFGLYTTLNWDTMEIERISYGVKTENPMTFFARLGTKVEHFVKNVPYGVDTQKMVYAAVTSSGEEYYKLQSYYRWRSVSRLNAAYIAARDKEST

SEQ ID NO: 70 - protein, 435 aa, Aspergillus versicolor
MTAPELRAPAGHPQEPPARSSPAQALSSYHHFPTSDQERWYQETGSLCSRFLEAGQYGLHQQYQFMFFFMHHLIPALGPYPQKWRSTISRSGLPIEFSLNFQKGSHRLLRIGFEPVNFLSGSSQDPFNRIPIADLLAQLARLQLRGFDTQCFQQLLTRFQLSLDEVRQLPPDDQPLKSQGAFGFDFNPDGAILVKGYVFPYLKAKAAGVPVATLIAESVRAIDADRNQFMHAFSLINDYMQESTGYNEYTFLSCDLVEMSRQRVKIYGAHTEVTWAKIAEMWTLGGRLIEEPEIMEGLARLKQIWSLLQIGEGSRAFKGGFDYGKASATDQIPSPIIWNYEISPGSSFPVPKFYLPVHGENDLRVARSLAQFWDSLGWSEHACAYPDMLQQLYPDLDVSRTSRLQSWISYSYTAKKGVYMSVYFHSQSTYLWEED

SEQ ID NO: 71 - protein, 472 aa, Aspergillus fumigatus Af293
MSIGAEIDSLVPAPPGLNGTAAGYPAKTQKELSNGDFDAHDGLSLAQLTPYDVLTAALPLPAPASSTGFWWRETGPVMSKLLAKANYPLYTHYKYLMLYHTHILPLLGPRPPLENSTHPSPSNAPWRSFLTDDFTPLEPSWNVNGNSEAQSTIRLGIEPIGFEAGAAADPFNQAAVTQFMHSYEATEVGATLTLFEHFRNDMFVGPETYAALRAKIPEGEHTTQSFLAFDLDAGRVTTKAYFFPILMSLKTGQSTTKVVSDSILHLALKSEVWGVQTIAAMSVMEAWIGSYGGAAKTEMISVDCVNEADSRIKIYVRMPHTSLRKVKEAYCLGGRLTDENTKEGLKLLDELWRTVFGIDDEDAELPQNSHRTAGTIFNFELRPGKWFPEPKVYLPVRHYCESDMQIASRLQTFFGRLGWHNMEKDYCKHLEDLFPHHPLSSSTGTHTFLSFSYKKQKGVYMTMYYNLRVYST

SEQ ID NO: 72 - protein, 440 aa, Aspergillus fumigatus
MDGEMTASPPDISACDTSAVDEQTGQSGQSQAPIPKDIAYHTLTKALLFPDIDQYQHWHHVAPMLAKMLVDGKYSIHQQYEYLCLFAQLVAPVLGPYPSPGRDVYRCTLGGNMTVELSQNFQRSGSTTRIAFEPVRYQASVGHDRFNRTSVNAFFSQLQLLVKSVNIELHHLLSEHLTLTAKDERNLNEEQLTKYLTNFQVKTQYVVALDLRKTGIVAKEYFFPGIKCAATGQTGSNACFGAIRAVDKDGHLDSLCQLIEAHFQQSKIDDAFLCCDLVDPAHTRFKVYIADPLVTLARAEEHWTLGGRLTDEDAAVGLEIIRGLWSELGIIQGPLEPSAMMEKGLLPIMLNYEMKAGQRLPKPKLYMPLTGIPETKIARIMTAFFQRHDMPEQAEVFMENLQAYYEGKNLEEATRYQAWLSFAYTKEKGPYLSIYYFWPE

SEQ ID NO: 73 - protein, 446 aa, Aspergillus oryzae RIB40
MSLRNDLDNGRPTKRLESWDIASMWLSDRKDEIQDWWDFSGPQLATLAHEAGYSTMTQIELLLFFRSVVLPRMGRFPDACRPRACAQSRSILTYDGSPIEYSWKWNNSANDHPEIRFCVEPVGDGLCADGIVGGKLRATDEILVQLAKRVPSTDLEWYHHFRDSFGLGHWTDGPLHEDAGTWQVRRPRMPVAFEFTPKGIVTKVYFTPPATLDDMPSFNMFADVVRPIGDKDTTALDESMEYLSRDPVGATLRPDVLAIDCISPLKSRIKLYAGTAMTTFTSAISVLTLGGRIPVTRHSIDEMWALFRMVLGLHDKFLQDEELPVQNPFQPSRAHPEDYYSGLLYYFNLAPGALLPDVKLYLPVIRYGRSDADIALGLQRFMASRHRGQYVDGFQRAMEIISQRHKSGNGHRIQTYIACSFDKDGSLSLTSYLNPGVYFSSETVDV

SEQ ID NO: 74 - protein, 424 aa, Aspergillus terreus NIH2624
MLPPSDSKDPRPWQILSQALGFPNYDQELWWQNTAETLNRVLEQCDYSVHLQYKYLAFYHKYILPSLGPFRRPGVEPEYISGLSHGGHPLEISVKIDKSKTICRLGLQAIGPLAGTARDPLNSFGDRELLKNLATLLPHVDLRLFDHFNAQVGLDRAQCAVATTKLIKESHNIVCTSLDLKDGEVIPKVYFSTIPKGLVTETPLFDLTFAAIEQMEVYHKDAPLRTALSSLKDFLRPRVPTDASITPPLTGLIGVDCIDPMLSRLKVYLATFRMDLSLIRDYWTLGGLLKDEGTMKGLEMVETLAKTLKLGDEACETLDAERLPFGINYAMKPGTAELAPPQIYFPLLGINDGFIADALVEFFQYMGWEDQASRYKDELKAKFPNVDISQTKNVHRWLGVAYSETKGPSMNIYYDVVAGNVARV

SEQ ID NO: 75 - protein, 459 aa, Aspergillus fumigatus
MKAANASSAEAYRVLSRAFRFDNEDQKLWWHSTAPMFAKMLETANYTTPCQYQYLITYKECVIPSLGCYPTNSAPRWLSILTRYGTPFELSLNCSNSIVRYTFEPINQHTGTDKDPFNTHAIWESLQHLLPLEKSIDLEWFRHFKHDLTLNSEESAFLAHNDRLVGGTIRTQNKLALDLKDGRFALKTYIYPALKAVVTGKTIHELVFGSVRRLAVREPRILPPLNMLEEYIRSRGSKSTASPRLVSCDLTSPAKSRIKIYLLEQMVSLEAMEDLWTLGGRRRDASTLEGLSLVRELWDLIQLSPGLKSYPAPYLPLGVIPDERLPLMANFTLHQNDPVPEPQVYFTTFGMNDMAVADALTTFFERRGWSEMARTYETTLKSYYPHADHDKLNYLHAYISFSYRDRTPYLSVYLQSFETGDWAVANLSESKVKCQDAACQPTALPPDLSKTGVYYSGLH

SEQ ID NO: 76 - protein, 463 aa, Aspergillus fumigatus
MPPAPPDQKPCHQLQPAPYRALSESILFGSVDEERWWHSTAPILSRLLISSNYDVDVQYKYLSLYRHLVLPALGPYPQRDPETGIIATQWRSGMVLTGLPIEFSNNVARALIRIGVDPVTADSGTAQDPFNTTRPKVYLETAARLLPGVDLTRFYEFETELVITKAEEAVLQANPDLFRSPWKSQILTAMDLQKSGTVLVKAYFYPQPKSAVTGRSTEDLLVNAIRKVDREGRFETQLANLQRYIERRRRGLHVPGVTADKPPATAADKAFDACSFFPHFLSTDLVEPGKSRVKFYASERHVNLQMVEDIWTFGGLRRDPDALRGLELLRHFWADIQMREGYYTMPRGFCELGKSSAGFEAPMMFHFHLDGSQSPFPDPQMYVCVFGMNSRKLVEGLTTYRRVGWEEMASHYQGNFLANYPDEDFEKAAHLCAYVSFAYKNGGAYVTLYNHSFNPVGDVSFPN

SEQ ID NO: 77 - protein, 437 aa, Aspergillus fischeri NRRL_181
MSPLSMQTDSVQGTAENKSLETNGTSNDQQLPWKVLGKSLGLPTIEQEQYWLNTAPYFNNLLIQCGYDVHQQYQYLAFYHRHVLPVLGPFIRSSAEANYISGFSAEGYPMELSVNYQASKATVRLGCEPVGEFAGTSQDPMNQFMTREVLGRLSRLDPTFDLRLFDYFDSQFSLTTSEANLAASKLIKQRRQSKVIAFDLKDGAIIPKAYFFLKGKSLASGIPVQDVAFNAIESIAPKQIESPLRVLRTFVTKLFSKPTVTSDVFILAVDCIVPEKSRIKLYVADSQLSLATLREFWTLGGSVTDSATMKGLEIAEELWRILQYDDAVCSHSNMDQLPLVVNYELSSGSATPKPQLYLPLHGRNDEAMANALTKFWDYLGWKGLAAQYKKDLYANNPCRNLAETTTVQRWVAFSYTESGGAYLTVYFHAVGGMKGNL

SEQ ID NO: 78 - protein, 470 aa, Xylona heveae TC161
MAPSMTANYPYSQISEFSKTIATSSDLDPNFGGGVSFKPSSCGGITTARKPWQILQDALGFRNEDEHFWWETTASVLGCLLEKAGYDVHLQYQYLSLYYRYVLPSYGPRPLQPGVPHWKSFMCDDFSPFEPSWNWDGSKSIIRFSFEPINRASGTSADPFNQIKPREVLAEISDISAGLDTQWYDHFAREFFLPSETASIIRSRLPEGEHMSQSFLAWDLNGGEASTKAYFFPILRSLETGRSTRDIVVDAITKLDSEKTSLRPSLTVLEDYMSSLPTEWQAKYEMIAIDCTDPSKSRIKIYVRMPSMAFNKVRDMYCLGGRLHGPNVDAAMKILDDLWPRVLYIPEGTGPDDELPSNTHRTAGAIFNFELKPGNPLPDPKLYLPVRHYAKSDLDIARGLQSFFRLQGWDEMADSYVEDLKNIFPTHDLANTAGSHTYLSYSYKKKTGAAVTMYYNPRIYECPPVVDEVF

SEQ ID NO: 79 - protein, 422 aa, Penicillium polonicum
MTYSTATPKDSTPVSLLSLYLTFRSKDDKLWWDNTAPVIGGFLAAAHYKVASQFEFLLFYHKYILPSLGHYPSPENEGDRWKSFLYRRGEPLELSFNYQKDSNCTVRLALEPVGPNAGTKDDPLNEFEAKILVEKIAQLDSNIDLQWVDFLDKEILLHNDELSQIKNTELEGSAHMSQRLVGVDFMSGGMKIKPYFVPWLKSLVTGVPTLQLMFQAIRKLDSVGSFSNGLSEVEAYLASTDQLLWSEENYLSFDCVDPGKSRIKLYVAEKVTCFNRIQSHWTLGGQLRSQANQEGLLLLKKLWNLLGYPGDPAQQTDRYLPFNFNWELRPSNPIPLPKVYFALGNEPDSLVSKALIGLFTELGWSDQIHAHKRSVEFAFPDCNLEETTHVLTWITVTYEEEKGAYITTYCNAIGGGHKLQFR

SEQ ID NO: 80 - protein, 453 aa, Aspergillus taichungensis
MLLSRTTSSQNPFHLLLSGTPRLPKMRPEQEPSIQAPSKKVPLPIADGDARPWQVLSLLLPFHNPDQKLWWDKVGPLIEIYLNCSGYNVGAQYRYLLMLHSIILPVLGPFPNSTRTHTSWPYFMNNGDPCDLSINYQGGSAPCVRLGIEPIGPMAGTNQDPMNEYAGRRLLEDLSRIQPGIDFQLFDHFRDTLTLSNYKARLCWHAVQEHGIKAQGHVALDLHEHSFKVKAYSIPLLRSLTSGVHYVRMMIDSIKMISRDQAITIGLSKVDEYLAATKHLLVDSRSCFSFDCADLQHSRYKIYVGANVKSLGEAYDFWTLGGRLKGEAIDRGFQLMETIWKTMYARSLPDRKPREYIPFIWNWEVSPTDSDPIPKAYFLVLNDYDILVSEVINCLFGELGWTEHAMTHQIIQKMAYPNHDFGSSTEIYSWISLAYSQSKGPYITIYSNPAASL

SEQ ID NO: 81 - protein, 355 aa, Trypanosoma grayi
MQLREELRDAVCVFYLVLRALDTVEDDMSLAVDLKLRELPVFHEHLRDPSWRMCGVGAGRERELLERFPHVTRVYARLGKAYQDVITDICARMASGMCEFLTRRVESRADYDLYCHYVAGLVGHGLTRLYVSGGFEDPNLADDLTNANHMGLFLQKTNIIRDFYEDICESPPRIFWPREIWAQYTDDLHAFKEEAHEAKALECLNAMVADALVHVPHVIEYMAALRDPSVFAFCAIPQLMAMATLALVFNNRNVFHSKVKLTRGSTCSIILYSTQLQSAMQTMRTQAQNLLARTGPDDVCYDKIAELVGEAVRAVDAHLQPETDGVARSMLTRYPALGGRLLYTLIDNVVGYLGK

SEQ ID NO: 82 - protein, 526 aa, Cutaneotrichosporon oleaginosum
MATLYPSIQSLQKFPYPGDGVVSSTLTDQHDTEGLIADVLDEQPPAHVPRLGLQNATTTLDSVNHLKFIQGAMMSLPSGFVGLDASRPWLVFWTVHSLDLLGVLLPQNIRDRAVSTILHFLHPTGGFCGGAANTHMPHLLPTYASVVSLAIVGNAGKGGGWERLVDARQDIYNFFMRCKRPDGGFVVGDNCEVDVRGTYCLLVVATLLDIITPELLHNVDKAIAAGQTFEGGFACSSFTFKDGNRVAMSEAHGGYTSCSVFSHFLLSSVQPPRRLESLPESFPVPIDVDSVVRWSAMMQGEAADGGGFRGRSNKLVDGCYSWWVGGTFPVLEELRRREAEVKTSPNGPTATKIVAVDDDGEDEWADEASMHALFNRGMCDSEVRLMAVALQEYTLLVAQSVTRGGLRDKPGKGPDLYHTCNNLSGLSVAQHRLTHTPEEVQKQREAFKADRGLPAVKPTTPGGGWKSEEERQAARREVWANVRAWVEDESDTLVVGGQMSQVNTTVPPFNMLEVRLQPFIDYFYCQ

SEQ ID NO: 83 - protein, 345 aa, Salpingoeca rosetta
MGYDGLVKLDPEQHLPYVTGGLGTLPSGFETLDASRPWLVYWSLNALVILGGTISPELKRRVINTLRMCQAETGGFGGGVGQVAHAAPTYAAVNALAIIGTEEAWSIINREKLASWLSSLIEDDGSMHMHDDGEIDVRAVYCGASAARLCGLDVDTIFAKCPQWVARCQTYEGGFAAIPGLEAHGGYTFCGFAAMSILCSTHLIDIPRLTEWLANRQMPMSGGFQGRPNKLVDGCYSFWVGGCFPILADLLEAQGLPGDVVNAEALIDYVVCVCQCPSGFRDKPGKRQDYYHTSYCLSGLASMKRFAPNHPILSQLNATHPIHNVPPANAERMIQAMSSQTTTRH

SEQ ID NO: 84 - protein, 307 aa, Streptomyces sp. Strain CL190 [sequence omitted]

SEQ ID NO: 85 - protein, 305 aa, Streptomyces sp. Act143
MSGAADVERVYAAMEEAAGLLGVTCAREKIYPLLTEFQDTLTDGVVVFSMASGRRSTELDFSISVPTSQGDPYATVVEKGLFPATGHPVDDLLADTQKHLPVSMFAIDGEVTGGFKKTYAFFPTDDMPGVAQLSAIPSMPSSVAENAELFARYGLDKVQMTSMDYKKRQVNLYFSELSEQTLAPESVLALVRELGLHVPTELGLEFCKRSFSVYPTLNWDTGKIDRLCFAVISTDPTLVPSTDERDIEQFRAYGTKAPYAYVGEKRTLVYGLTLSPTEEYYKLGAYYHITDIQRRLLKAFDALED

SEQ ID NO: 86 - protein, 344 aa, Streptomyces antibioticus
MTSRVCSTSQRQSILQRGSRPMAEAEARTDRQDRSVEVCMSGAADVERVYAAMEEAAGLLGVTCAREKIYPLLTEFQDTLTDGVVVFSMASGRRSTELDFSISVPTSQGDPYATVVDKGLFPATGHPVDDLLADTQKHLPVSMFAIDGEVTGGFKKTYAFFPTDDMPGVAQLSAIPSMPSSVAENAELFARYGLDKVQMTSMDYKKRQVNLYFSELSEQTLAPESVLALVRELGLHVPTELGLEFCKRSFSVYPTLNWDTGKIDRLCFAVISTDPTLVPSTDERDIEQFRHYGTKAPYAYVGENRTLVYGLTLSPTEEYYKLGAYYHITDIQRRLLKAFDALED

SEQ ID NO: 87 - protein, 305 aa, Streptomyces antibioticus
MSGAADVERVYAAMEEAAGLLGVTCAREKIYPLLTEFQDTLTDGVVVFSMASGRRSTELDFSISVPTSQGDPYATVVDKGLFPATGHPVDDLLADTQKHLPVSMFAIDGEVTGGFKKTYAFFPTDDMPGVAQLSAIPSMPSSVAENAELFARYGLDKVQMTSMDYKKRQVNLYFSELSEQTLAPESVLALVRELGLHVPTELGLEFCKRSFSVYPTLNWDTGKIDRLCFAVISTDPTLVPSTDERDIEQFRHYGTKAPYAYVGENRTLVYGLTLSPTEEYYKLGAYYHITDIQRRLLKAFDALED

SEQ ID NO: 88 - protein, 309 aa, Actinobacteria bacterium OV320
MEVSMSGAADVERVYAAMEEAAGLLDVSCAREKIYPLLTVFQDTLTDGVVVFSMASGRRSTELDFSISVPVSQGDPYATVVREGLFRATGSPVDELLADTVKHLPVSMFAIDGEVTGGFKKTYAFFPTDDMPGVAQLTGIPSMPASVAENAELFARYGLDKVQMTSMDYKKRQVNLYFSDLKQEYLQPEAVVALARELGLQVPGELGLEFCKRSFAVYPTLNWDTGKIDRLCFAAISTDPTLVPSTDERDIEMFREYATKAPYAYVGEKRTLVYGLTLSPTEEYYKLGAYYHITDIQRQLLKAFDALED

SEQ ID NO: 89 - protein, 309 aa, Streptomyces sp. Root1310
MEVSMSGAADVERVYAAMEEAAGLLDVSCAREKIYPLLTVFQDTLTDGVVVFSMASGRRSTELDFSISVPVSQGDPYATVVKEGLFQATGSPVDELLADTVAHLPVSMFAIDGEVTGGFKKTYAFFPTDDMPGVAQLAAIPSMPASVAENAELFARYGLDKVQMTSMDYKKRQVNLYFSDLKQEYLQPESVVALARELGLRVPGELGLEFCKRSFAVYPTLNWDTGKIDRLCFAAISTDPTLVPSEDERDIEMFRNYATKAPYAYVGEKRTLVYGLTLSSTEEYYKLGAYYHITDIQRQLLKAFDALED

SEQ ID NO: 90 - protein, 305 aa, Streptomyces sp. Root1310
MSGAADVERVYAAMEEAAGLLDVSCAREKIYPLLTVFQDTLTDGVVVFSMASGRRSTELDFSISVPVSQGDPYATVVKEGLFQATGSPVDELLADTVAHLPVSMFAIDGEVTGGFKKTYAFFPTDDMPGVAQLAAIPSMPASVAENAELFARYGLDKVQMTSMDYKKRQVNLYFSDLKQEYLQPESVVALARELGLRVPGELGLEFCKRSFAVYPTLNWDTGKIDRLCFAAISTDPTLVPSEDERDIEMFRNYATKAPYAYVGEKRTLVYGLTLSSTEEYYKLGAYYHITDIQRQLLKAFDALED

SEQ ID NO: 91 - protein, 305 aa, Actinobacteria bacterium OV320
MSGAADVERVYAAMEEAAGLLDVSCAREKIYPLLTVFQDTLTDGVVVFSMASGRRSTELDFSISVPVSQGDPYATVVREGLFRATGSPVDELLADTVKHLPVSMFAIDGEVTGGFKKTYAFFPTDDMPGVAQLTGIPSMPASVAENAELFARYGLDKVQMTSMDYKKRQVNLYFSDLKQEYLQPEAVVALARELGLQVPGELGLEFCKRSFAVYPTLNWDTGKIDRLCFAAISTDPTLVPSTDERDIEMFREYATKAPYAYVGEKRTLVYGLTLSPTEEYYKLGAYYHITDIQRQLLKAFDALED

SEQ ID NO: 92 - protein, 305 aa, Streptomyces tendae
MSGAADVERVYAAMEEAAGLLDVSCAREKIYPLLTVFQDTLTDGVVVFSMASGRRSTELDFSISVPVSQGDPYATVVKEGLFRATGSPVDELLADTVKHLPVSMFAIDGEVTGGFKKTYAFFPTDDMPGVAQLTEIPSMPASVAENAELFARYGLDKVQMTSMDYKKRQVNLYFSDLKQEYLQPEAVVALARELGLQVPGELGLEFCKRSFAVYPTLNWDTGKIDRLCFAAISTDPTLVPSTDERDIEMFREYATKAPYAYVGEKRTLVYGLTLSSTEEYYKLGAYYHITDIQRQLLKAFDALED

SEQ ID NO: 93 - protein, 305 aa, Streptomyces sp. URHA0041
MSGAAEVERVYSAMEESAGLLDVACSREKIQPILTAFQDVLADGVIVFSMANGRHATELDFSISVPAGHGDPYAAALEHGLIPATGHPVGDLLADTQKALPVSMFAVDGEVTSGFKKTYAFFPTDDMPGLAQLIDIPSMPPSVAENAELFGRYGLDKVQMISLDYKKNQVNLYFSNLNPEFLQPEPVQAMVREMGLQLPADKGLAFAKRSFAVYPTLSWDSAKIERLCFAVISTDPTLAPAQEQADLDLFSTYANNAPYAYAGEKRTLVYGLTLSPSEEYYKLGSYYQISDIQRKLLKAFDALTD

SEQ ID NO: 94 - protein, 305 aa, Streptomyces paucisporeus
MSGAAEVERVYSAMEEAAGLLDVACSPEKVRPILTAFQDVLSDGVIVYSMASGRHATELDFSISVPADHGDPYTAALAHGLIPETDHPVGNLLADTQKALPVSMFAVDGEVTGGFKKTYAFFPTDDMPGLAQLIDIPSMPPSVAENAELFARYGLDKVQMTSLDYKRKQVNLYFSNLQPEFLAPEPVLSMVREMGLELPGEKGLKFARRSFAIYPTLGWESGKIERLCFAVISTDPGLVPAPDEADRALFSTYANNAPYAYAGEKRTLVYGLTLSPTEEYYKLGSYYQITDIQRTLLKAFDALTD

SEQ ID NO: 95 - protein, 544 aa, Cannabis sativa
MKCSTFSFWFVCKIIFFFFSFNIQTSIANPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 96 - protein, 545 aa, Cannabis sativa
MKCSTFCFWYVCKIIFFFLSFNIQISIANPQENFLKCFSQYIPTNVTNAKLVYTQHDQFYMSILNSTIQNLRFTSDTTPKPLVIITPLNVSHIQGTILCSKKVGLQIRTRSGGHDAEGMSYISQVPFVIVDLRNMHSVKIDVHSQTAWVEAGATLGEVYYWINENNENLSFPAGYCPTVGAGGHFSGGGYGALMRNYGLAADNIIDAHLVNVDGKVLDRKSMGEDLFWAIRGGGGENFGIIAAWKIRLVAVPSMSTIFSVKKNMEIHELVKLVNKWQNIAYMYEKELLLFTHFITRNITDNQGKNKTTIHSYFSSIFHGGVDSLVDLMNKSFPELGIKKTDCKQLSWIDTIIFYSGVVNYNTTYFKKEILLDRSGGRKAAFSIKLDYVKKPIPETAMVTILEKLYEEDVGVGMFVFYPYGGIMDEISESAIPFPHRAGIMYEIWYIASWEKQEDNEKHINWIRNVYNFTTPYVSQNPRMAYLNYRDLDLGKTNFESPNNYTQARIWGEKYFGKNFNRLVKVKTKVDPDNFFRNEQSIPPLPLRHH

SEQ ID NO: 97 - protein, 545 aa, Cannabis sativa
MKCSTFCFWYVCKIIFFFLSFNIQISIANPQENFLKCLSQYIPTNVTNAKLVYTQHDQFYMSILNSTVQNLRFTSDTTPKPLVITTPLNVSHIQGTILCSKKVGLQIRTRSGGHDAEGMSYISQVPFVIVDLRNMHSVKIDVHSQTAWVESGATLGEVYYWINENNENLSFPAGYCPTVGTGGHFSGGGYGALMRNYGLAADNIIDAHLVNVDGKVLDRKSMGEDLFWAIRGGGGENFGIIAAWKIRLVAVPSMSTIFSVKKNMEIHELVKLVNKWQNIAYMYEKELLLFTHFITRNITDNQGKNKTTIHSYFSSIFHGGVDSLVDLMNKSFPELGIKKTDCKQLSWIDTIIFYSGVVNYNTTNFKKEILLDRSGGRKAAFSIKLDYVKKPIPETAMVTILEKLYEEDVGVGMFVFYPYGGIMDEISESAIPFPHRAGITYEIWYIASWEKQEDNEKHINWIRNVYNFTTPYVSQNPRMAYLNYRDLDLGKTNFESPNNYTQARIWGEKYFGKNFNRLVKVKTKVDPDNFFRNEQSIPPLPLRHH

SEQ ID NO: 98 - protein, 545 aa, Cannabis sativa
MNCSTFSFWFVCKIIFFFLSFNIQISIANPQENFLKCFSEYIPNNPANPKFIYTQHDQLYMSVLNSTIQNLRFTSDTTPKPLVIVTPSNVSHIQASILCSKKVGLQIRTRSGGHDAEGLSYISQVPFAIVDLRNMHTVKVDIHSQTAWVEAGATLGEVYYWINEMNENFSFPGGYCPTVGVGGHFSGGGYGALMRNYGLAADNIIDAHLVNVDGKVLDRKSMGEDLFWAIRGGGGENFGIIAACKIKLVVVPSKATIFSVKKNMEIHGLVKLFNKWQNIAYKYDKDLMLTTHFRTRNITDNHGKNKTTVHGYFSSIFLGGVDSLVDLMNKSFPELGIKKTDCKELSWIDTTIFYSGVVNYNTANFKKEILLDRSAGKKTAFSIKLDYVKKLIPETAMVKILEKLYEEEVGVGMYVLYPYGGIMDEISESAIPFPHRAGIMYELWYTATWEKQEDNEKHINWVRSVYNFTTPYVSQNPRLAYLNYRDLDLGKTNPESPNNYTQARIWGEKYFGKNFNRLVKVKTKADPNNFFRNEQSIPPLPPRHH

SEQ ID NO: 99 - protein, 545 aa, Cannabis sativa
MNCSAFSFWFVCKIIFFFLSFHIQISIANPRENFLKCFSKHIPNNVANPKLVYTQHDQLYMSILNSTIQNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHH

SEQ ID NO: 100 - protein, 553 aa, Actinidia chinensis var. chinensis
MQKHKNLKTYKMKTPTTLLSFAFVVLFLFSFSWGALAQNHEDFLQCLSLHSQNSTSITKVIYTPNNSSYLSVLNFSIKNLRFTSPSTPKPLVIVTPLDESQIQSTIYCAKTHGMEIRTRSGGHDFEGLSYISEVSFVILDLINLHSIVVDSENGTAWVQSGATIGQLYYRIAEKSRNYGFPAGGCPTVGVGGHFSGGGYGMMLRKYGLAADNVVDARIIDVNGNILDRKSMGEDLFWAIRGGGGASFGVIVAWKINLVVVPSKVTVFTINRTLEQNATNLIHKWQSIAHKFPQELLVAILIKRVDSSHDNGEDTMQAFFTSLYLGGIDQLIPLMQESFPELGLTREDCTEMSWIESILYFAGFPSGSSLDVLLNRTQLSTRYFKAKSDYVKEPIPLFGWKGIWDLFFKDEGELAEMALIPYGGKMNEISESSIPFPHRAGNLYKILHMVYWDEEGAEESEKHISWIRKLYSYMAPYVSKFPRAAYINYRDLDVGVNNKNGNTSYAQASIWGMKYFKNNFNRLVHVKTKVDPSNFFKNEQSIPTLPSWWKKRGN

SEQ ID NO: 101 - protein, 533 aa, Populus trichocarpa
MTCLKASMLPFLLCLLISFSWVISAHPREDFLKCLSLHFEDPAAMSNAIHTPYNSSYSSILQFSIRNLRFNSSELKPLVIVTPTNASHIQAAILCSQRHNLQIRIRSGGHDFEGLSYMAALPFVIIDLISLRAVNVDATSRTAWVQAGATLGELYYSISEKSRTLAFPAGSCPTIGVGGHFSGGGHGTMVRKFGLASDNVIDAHLIDSKGRILDRASMGEDLFWAIRGGGGQSFGVVVAWKISLVEVPSTVTMFSVSRTLEQNATKLLHRWQYVANTLPEDLVIDVQVTRVNSSQEGNTTIQATFFSLFLGEVDQLLPVMQESFPELGLVKDDCFEMSWIESVFYTGGFTSNASLDVLLNRTPRSIPRFKAKSDYVKEPMPEIAFEGIWERFFEEDIEAPTLILIPYGGKMDEISESSTPFPHRAGNLYVLVSSVSWREESKEASRRHMAWIRRLYSYLTKYVSKNPREAYVNYRDLDLGINNLTGTTSYKQASIWGRKYFKNNFDRLVRVKTEVDPTNFFRNEQSIPSLSSW

(19) United States
(12) Patent Application Publication
(10) Pub. No.: US 2022/0002764 A1
(43) Pub. Date: Jan. 6, 2022

(54) MICROBIAL CELLS AND METHODS FOR PRODUCING CANNABINOIDS

(71) Applicant: Manus Bio Inc., Cambridge, MA (US)

(72) Inventors: Ryan A. PHILIPPE, Somerville, MA (US); Ajikumar Parayil KUMARAN, Watertown, MA (US); Christine Nicole S. SANTOS, Newton, MA (US); Lu CHEN, Cambridge, MA (US)

(21) Appl. No.: 17/293,230

(22) PCT Filed: Nov. 14, 2019
(86) PCT No.: PCT/US2019/061487

§ 371 Date: May 12, 2021

Related U.S. Application Data
(60) Provisional application No. 62/767,056, filed on Nov. 14, 2018.

(51) Int. Cl. C12P 7/42; C12N 9/12; C12N 15/52; C12N 1/16; C12N 1/32; C12N 1/20; C12N 9/02; C12N 9/10; C12N 9/88; C12N 9/00

(52) U.S. Cl. CPC C12P 7/42; C12Y 205/01001; C12N 15/52; C12N 1/16; C12N 1/32; C12N 1/20; C12N 9/0004; C12Y 121/03008; C12Y 121/03007; C12N 9/1029; C12Y 203/01206; C12N 9/88; C12Y 404/01026; C12N 9/93; C12N 9/1085; C12N 9/12

(57) ABSTRACT

Enzymes involved in cannabinoid biosynthesis are recombinantly expressed in a host cell. The host cell may be a prokaryote (e.g. Escherichia coli) or a eukaryote (e.g. Yarrowia lipolytica). The enzymes include a heterologous cannabigerolic acid synthase as well as additional enzymes involved in the biosynthesis of cannabinoid precursors such as geranyl diphosphate, olivetol, olivetolic acid, divarin and/or divarinic acid. Methods are provided for producing C5-cannabinoids and/or C3-cannabinoids by fermentation of the recombinant host cell. Alternatively, cannabinoids can be produced by biotransformation of cannabinoid precursors in recombinant cells or by disrupted recombinant cells.

### RELATED APPLICATIONS

This application claims the benefit of and priority to U.S. Provisional Patent Application No. 62/767,056, filed Nov. 14, 2018, the entire contents of all of which are hereby incorporated by reference in their entirety.

### SEQUENCE LISTING

The application contains a Sequence Listing which has been submitted in ASCII format via EFS-Web and is hereby incorporated by reference in its entirety. Said ASCII copy, created on Nov. 13, 2019, is named MAN-021PC_Sequence_Listing.txt and is 393,114 bytes in size.

### BACKGROUND

Cannabis sativa (cannabis) is a flowering plant that has been cultivated for over 10,000 years. It is best known as a source for cannabinoids with psychoactive effects, such as tetrahydrocannabinol (THC). Cannabis is an annual, usually dioecious wind-pollinated herb, with male and female flowers growing on separate plants. Cannabinoids are found throughout the plant, with the exception of its seeds, but are mainly concentrated in the glandular trichomes of female flowers.

The beneficial properties of less-abundant natural cannabinoids have been discovered more recently. Cannabidiol (CBD), for example, has been investigated for the treatment of a variety of ailments, and has been approved by the Federal Drug Administration (FDA) for the treatment of seizures associated with two rare and severe forms of epilepsy: Lennox-Gastaut syndrome and Dravet syndrome. Additional potentially useful cannabinoids include cannabinol (CBN), a non-psychoactive cannabinoid with promise as a sedative and sleep aid; Δ8-THC, an isomer being investigated for treatment of the nausea associated with chemotherapy; and Tetrahydrocannabivarin (THCV), which has energizing and appetite suppressing activities.

Given the recognized and potential value of these and other rare cannabinoids, cost effective, scalable, and/or sustainable processes are needed for their production.

### SUMMARY

The present invention is concerned with the production of cannabinoids. In various aspects, the invention provides enzymes for cannabinoid biosynthesis, polynucleotides encoding said enzymes, recombinant host cells expressing said enzymes, and recombinant host cells that produce cannabinoids. In other aspects, the invention provides methods of producing cannabinoids using the enzymes or host cells. For example, cannabinoids may be produced by fermentation of recombinant host cells, or by biotransformation of cannabinoid precursors by whole cells, disrupted cells, or isolated or partially purified enzymes. Isolated cannabinoids produced according to the present invention may have higher purity and/or yield than natural cannabinoids because recombinant cells can be engineered to produce specific cannabinoid compounds by expressing particular biosynthetic enzymes. The cannabinoids thus produced may be incorporated into products such as pharmaceuticals, dietary supplements, baked goods, and others.

In some embodiments, the present invention provides methods, enzymes, and recombinant host cells for producing cannabinoids such as Δ9-tetrahydrocannbinol (THC or Δ9-THC), cannabigerol (CBG), cannabicyclol (CBL), cannabidiol (CBD), cannabinol (CBN), cannabichromene (CBC), Δ8-tetrahydrocannbinol (Δ8-THC), cannabinerol (CBNR), Δ9-tetrahydrocannabivarol (THCV), cannabidivarin (CBDV) and/or cannabichrovarin (CBCV), as well as derivatives thereof. In some embodiments, recombinant host cells are fed with a cannabinoid biosynthetic intermediate, such as olivetol, olivetolic acid (OA), divarin, divarinic acid (DA), hexanoic acid, butyric acid, hexanoyl-CoA, butyryl-CoA, GPP precursor, or derivative thereof. Alternatively, host cells produce the cannabinoid from C1-C6 carbon substrates, such as glucose. In some embodiments, cannabinoids are recovered from recombinant host cells or their culture medium.

In some embodiments, the host cell recombinantly expresses a prenylating enzyme having cannabigerolic acid synthase (CBGAS) and/or cannabigerovarinic acid synthase (CBGVAS) activity, central enzymes for the biosynthesis of all cannabinoids, and one or more additional enzymes, such as geranyl diphosphate synthase (GPPS), acyl-activating enzyme (AAE), olivetol synthase (OLS), olivetolic acid cyclase (OAC), divarin synthase (DS), divaric acid cyclase (DAS), that increase the availability of CBGAS reactants. The host cell may also express enzymes such as tetrahydrocannabinolic acid synthase (THCAS), cannabidiolic acid synthase (CBDAS), and cannabichromenic acid synthase (CBCAS), that act on CBGAS and/or CBGVAS products. In some embodiments, one or more of the enzymes expressed in the host cell is derived from a cannabinoid-producing plant such as Cannabis sativa.

In some embodiments, the host cell further expresses or overexpresses one or more enzymes in the methylerythritol phosphate (MEP) and/or the mevalonic acid (MVA) pathway to catalyze the conversion of glucose to isopentenyl pyrophosphate (IPP) and/or dimethylallyl pyrophosphate (DMAPP). In some embodiments, the host cell further expresses an enzyme catalyzing the conversion of IPP and/or DMAPP to geranyl diphosphate (GPP), allowing for one or more cannabinoids to be produced from sugar or other carbon sources (carbon substrates such as C1, C2, C3, C4, C5, and/or C6 carbon substrates). In some embodiments, the host cell may express one or more enzymes capable of converting isoprenol to IPP and/or prenol to DMAPP.

In some embodiments, the host cell is engineered for increased synthesis of cannabinoid precursors. In some embodiments, the host cell is engineered for decreased utilization of cannabinoid precursors by competing biosynthetic pathways. The host cell may be engineered to increase carbon flux through the MEP pathway or for increased production of acetyl-CoA, malonyl-CoA, fatty acids, and/or other biomolecules.

In some embodiments, the host cell is a microbial cell, which may be prokaryotic or a eukaryotic (e.g. a bacterium or a yeast). For example, the host cell may be an Escherichia coli, Saccharomyces cerevisiae or Yarrowia lipolytica cell.

Other aspects and embodiments of the invention will be apparent from the following detailed disclosure.

### BRIEF DESCRIPTION OF THE FIGURES

FIG. 1 provides examples of cannabinoids. Compound abbreviations: THC, Δ9-tetrahydrocannbinol; CBG, cannabigerol; CBD, cannabidiol; CBC, cannabichromene; CBNR, cannabinerol; CBL, cannabicyclol; CBN, cannabinol; Δ8-THC, Δ8-tetrahydrocannbinol; THCV, Δ9-tetrahydrocannabivarol; CBDV, cannabidivarin; CBCV cannabichrovarin.

FIG. 2 shows the C5 cannabinoid biosynthetic pathway. CBD is produced via nonenzymatic conversion from CBDA, whose precursor compound is CBGA produced from two precursors, GPP and olivetolic acid. These precursors are produced by the terpenoid pathway and fatty acid-based polyketide pathway, respectively. Terpenoid precursors can be obtained from the MEP or MVA pathways. Enzyme abbreviations: AAE, acyl activating enzyme (or hexanoyl-CoA synthetase); GPPS, geranyl diphosphate synthase; OLS, olivetol synthase; OAC, olivetolic acid cyclase; CBGAS, cannabigerolic acid synthase; CBCAS, cannabichromic acid synthase; CBDAS, cannabidiolic acid synthase; THCAS, tetrahydrocannabinolic acid synthase. Compound abbreviations: G3P, glyceraldehyde 3-phosphate; IPP, isopentenyl diphosphate; DMAPP, dimethyl allyl diphosphate; GPP, geranyl diphosphate; CBGA, cannabigerolic acid; CBCA, cannabichromic acid; CBDA, cannabidiolic acid; THCA, tetrahydrocannabinolic acid; CBC, cannabichromene; CBD, cannabidiol; THC, tetrahydrocannabinol.

FIG. 3 shows the C3-cannabinoid biosynthetic pathway. The pathway is analogous to the C5-cannabinoid pathway, but proceeds through divarinic acid in lieu of olivetolic acid. Enzymes accept the precursor with the shorter side chains and proceed with the same enzyme reactions on the alternate substrate. Enzymes abbreviations: AAE, acyl-activating enzyme; DS, divarin synthase; DAC, divarinic acid cyclase; CBGAS, cannabigerolic acid synthase; CBCAS, cannabichromenic acid synthase; CBDAS, cannabidiolic acid synthase; THCAS, tetrahydrocannabinolic acid synthase. Compound abbreviations: GPP, geranyl diphosphate; CBGVA, cannabigerovarinic acid; CBCVA, cannabichrovarinic acid; CBDA, cannabidivarinic acid; THCVA, tetrahydrocannabivarinic acid; CBCV, cannabichrovarin; CBDV, cannabidivarin; THCV, tetrahydrocannabivarin.

FIG. 4 shows liquid chromatography (LC) mass spectrometry MS/MS analysis of prenyltransferase enzymatic assays to generate cannabigerolic acid (CBGA) product. FIG. 4A shows an authentic CBGA standard. FIG. 4B shows control with no enzyme. FIG. 4C shows a representative enzyme A. FIG. 4D shows a representative enzyme B. FIG. 4E shows a representative enzyme C generating side product 1 (SP1) as the main product.

### DETAILED DESCRIPTION

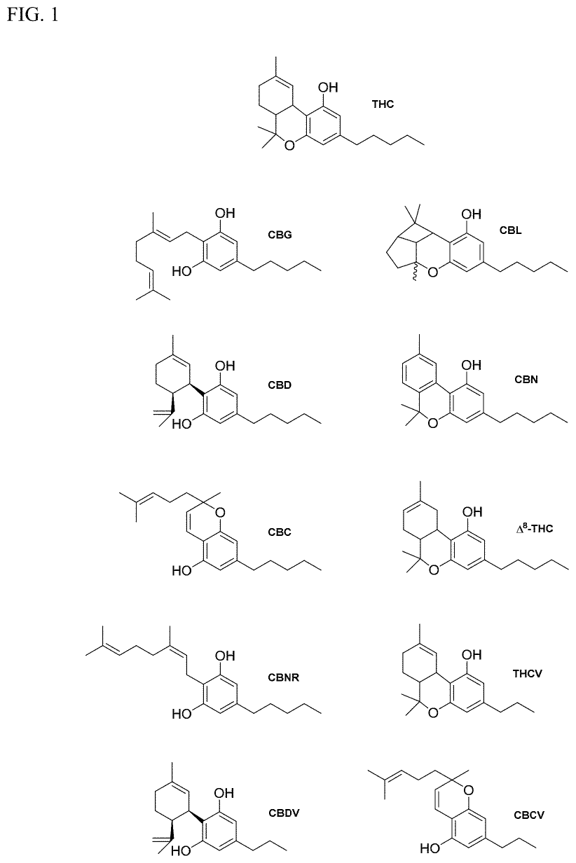

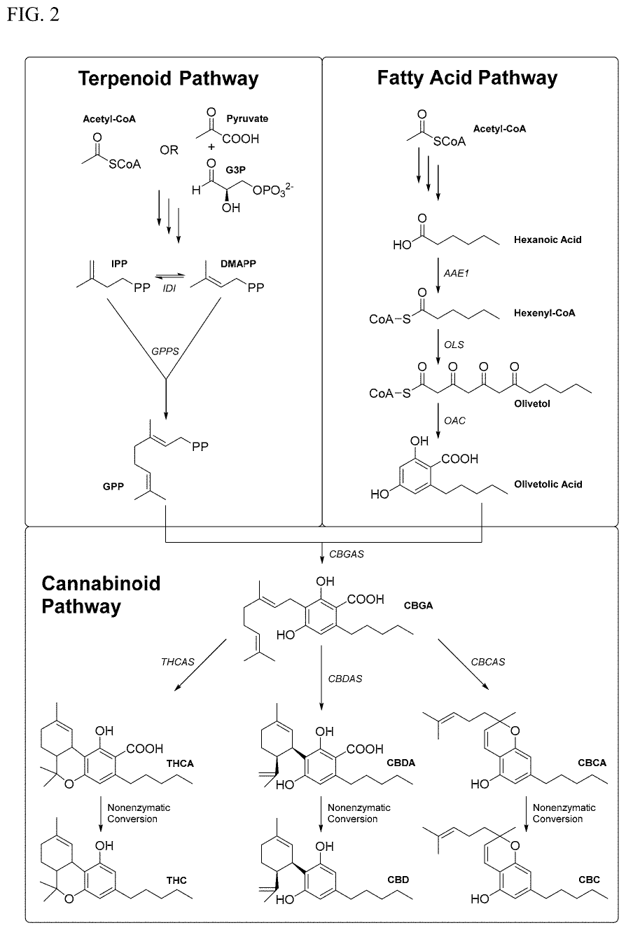

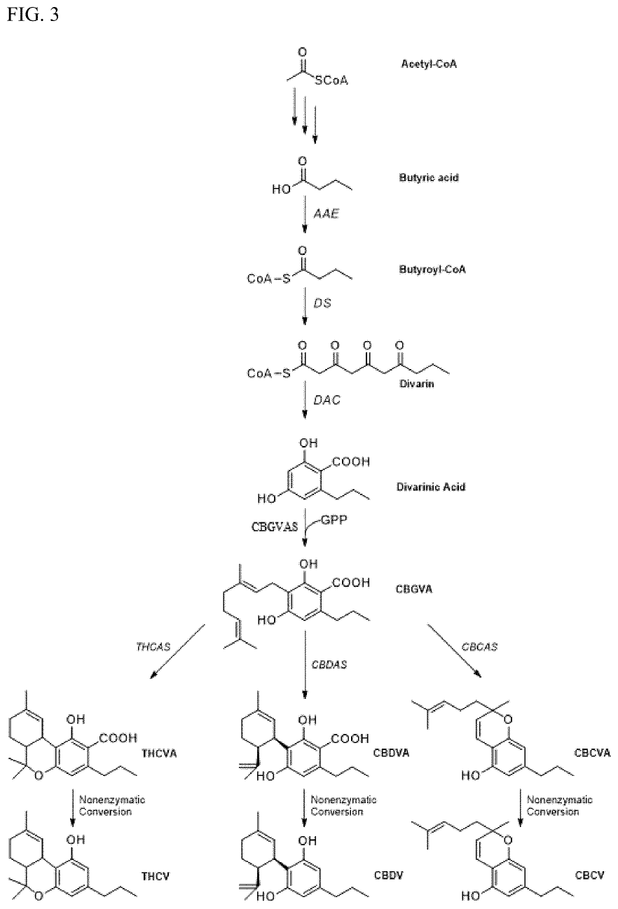

The structures of various cannabinoids produced in the female flowers of Cannabis sativa are shown in FIG. 1. These compounds can be produced from one of two possible intermediates: either cannabigerolic acid (CBGA) for the C5-cannabinoids or cannabigerovarinic acid (CBGVA) for the C3-cannabinoids. FIGS. 2 and 3. The primary difference between the C5- and C3-pathways is that olivetolic acid (OA) is the precursor for C5-cannabinoids whereas divaric acid (DA) is the precursor for C3-cannabinoids. The central enzyme in both pathways is a prenyl transferase, cannabigerolic acid synthase (CBGAS) or cannabigerovarinic acid synthase (CBGVAS), respectively, that adds a geranyl diphosphate (GPP) to either OA or DA. The resulting products are then cyclized at different positions by THCAS, CBDAS, or CBCAS. After cyclization, further transformations to active compounds such as THC occur by non-enzymatic decarboxylation in the presence of heat or ultraviolet light.

In accordance with various embodiments, the invention provides a microbial cell for producing one or more cannabinoids, where the microbial cell expresses a cannabinoid biosynthetic pathway that comprises a heterologous prenyltransferase having cannabigerolic acid synthase (CBGAS) activity or cannabigerovarinic acid synthase (CBGVAS) enzyme. The microbial cell further comprises one or more modifications that increase carbon flux to geranyl diphosphate (GPP) and/or carbon flux to hexanoic acid, hexanoyl-CoA, butyric acid, butyryl-CoA, and/or acetyl-CoA. Alternatively, or in addition to comprising one or more modifications that increase carbon flux to GPP, the microbial cell produces the cannabinoid from a fed precursor selected from olivetol, olivetolic acid, divarin, divarinic acid, hexanoic acid, butyric acid, hexanoyl-CoA, butyryl-CoA, GPP precursor, or derivative thereof.

CBGAS, also known as geranylpyrophosphate:olivetolate geranyltransferase, is a prenyl transferase that catalyzes the C-prenylation of OA or DA (CBGVAS activity) using GPP. In some embodiments, the CBGAS or CBGVAS enzyme may be Cannabis sativa CBGAS having SEQ ID NO: 60, or a derivative thereof. Alternatively, the prenyl transferase activity may be provided by an enzyme comprising an amino acid sequence selected from SEQ ID NOs: 61 to 94, or a derivative thereof. In some embodiments, the derivative comprises an amino acid sequence having at least 60%, at least 70%, at least 80%, at least 90%, at least 95%, or at least 98% identity to an amino acid sequence selected from SEQ ID NOS: 60 to 94. In some embodiments, the derivative comprises an amino acid sequence having from 1 to 20 or from 1 to 10 amino acid modifications with respect to a sequence selected from SEQ ID NOS: 60 to 94. Amino acid modifications can be independently selected from substitutions, deletions, and insertions.

In some embodiments, the prenyl transferase activity may be provided by an enzyme comprising an amino acid sequence of SEQ ID NO: 63, or a derivative thereof. In some embodiments, the derivative comprises an amino acid sequence having at least 60%, at least 70%, at least 80%, at least 90%, at least 95%, or at least 98% identity to the amino acid sequence of SEQ ID NO: 63. In some embodiments, the derivative comprises an amino acid sequence having from 1 to 20 or from 1 to 10 amino acid modifications with respect to the sequence of SEQ ID NO: 63. Amino acid modifications can be independently selected from substitutions, deletions, and insertions.

In some embodiments, the prenyl transferase activity may be provided by an enzyme comprising an amino acid sequence of SEQ ID NO: 74, or a derivative thereof. In some embodiments, the derivative comprises an amino acid sequence having at least 60%, at least 70%, at least 80%, at least 90%, at least 95%, or at least 98% identity to the amino acid sequence of SEQ ID NO: 74. In some embodiments, the derivative comprises an amino acid sequence having from 1 to 20 or from 1 to 10 amino acid modifications with respect to the sequence of SEQ ID NO: 74. Amino acid modifications can be independently selected from substitutions, deletions, and insertions.

In some embodiments, the prenyl transferase activity may be provided by an enzyme comprising an amino acid sequence of SEQ ID NO: 77, or a derivative thereof. In some embodiments, the derivative comprises an amino acid sequence having at least 60%, at least 70%, at least 80%, at least 90%, at least 95%, or at least 98% identity to the amino acid sequence of SEQ ID NO: 77. In some embodiments, the derivative comprises an amino acid sequence having from 1 to 20 or from 1 to 10 amino acid modifications with respect to the sequence of SEQ ID NO: 77. Amino acid modifications can be independently selected from substitutions, deletions, and insertions.

In some embodiments, the prenyl transferase activity may be provided by an enzyme comprising an amino acid sequence of SEQ ID NO: 84, or a derivative thereof. In some embodiments, the derivative comprises an amino acid sequence having at least 60%, at least 70%, at least 80%, at least 90%, at least 95%, or at least 98% identity to the amino acid sequence of SEQ ID NO: 84. In some embodiments, the derivative comprises an amino acid sequence having from 1 to 20 or from 1 to 10 amino acid modifications with respect to the sequence of SEQ ID NO: 84. Amino acid modifications can be independently selected from substitutions, deletions, and insertions. In some embodiments, the derivative comprises a mutation at position corresponding to G286 of SEQ ID NO: 84. In some embodiments, the mutation at the position corresponding to G286 with respect to SEQ ID NO: 84 is a substitution with a polar amino acid. In embodiments, the substitution at position corresponding to G286 with respect to SEQ ID NO: 84 is selected from Arginine, Asparagine, Aspartic acid, Glutamine, Glutamic acid, Histidine, Lysine, Serine, Threonine, and Tyrosine. In one embodiment, the substitution at position corresponding to G286, with respect to SEQ ID NO: 84, is Serine.

In some embodiments, the prenyl transferase activity may be provided by an enzyme comprising an amino acid sequence of SEQ ID NO: 85, or a derivative thereof. In some embodiments, the derivative comprises an amino acid sequence having at least 60%, at least 70%, at least 80%, at least 90%, at least 95%, or at least 98% identity to the amino acid sequence of SEQ ID NO: 85. In some embodiments, the derivative comprises an amino acid sequence having from 1 to 20 or from 1 to 10 amino acid modifications with respect to the sequence of SEQ ID NO: 85. Amino acid modifications can be independently selected from substitutions, deletions, and insertions.

In some embodiments, the prenyl transferase activity may be provided by an enzyme comprising an amino acid sequence of SEQ ID NO: 86, or a derivative thereof. In some embodiments, the derivative comprises an amino acid sequence having at least 60%, at least 70%, at least 80%, at least 90%, at least 95%, or at least 98% identity to the amino acid sequence of SEQ ID NO: 86. In some embodiments, the derivative comprises an amino acid sequence having from 1 to 20 or from 1 to 10 amino acid modifications with respect to the sequence of SEQ ID NO: 86. Amino acid modifications can be independently selected from substitutions, deletions, and insertions.

In some embodiments, the prenyl transferase activity may be provided by an enzyme comprising an amino acid sequence of SEQ ID NO: 87, or a derivative thereof. In some embodiments, the derivative comprises an amino acid sequence having at least 60%, at least 70%, at least 80%, at least 90%, at least 95%, or at least 98% identity to the amino acid sequence of SEQ ID NO: 87. In some embodiments, the derivative comprises an amino acid sequence having from 1 to 20 or from 1 to 10 amino acid modifications with respect to the sequence of SEQ ID NO: 87. Amino acid modifications can be independently selected from substitutions, deletions, and insertions.

In some embodiments, the prenyl transferase activity may be provided by an enzyme comprising an amino acid sequence of SEQ ID NO: 88, or a derivative thereof. In some embodiments, the derivative comprises an amino acid sequence having at least 60%, at least 70%, at least 80%, at least 90%, at least 95%, or at least 98% identity to the amino acid sequence of SEQ ID NO: 88. In some embodiments, the derivative comprises an amino acid sequence having from 1 to 20 or from 1 to 10 amino acid modifications with respect to the sequence of SEQ ID NO: 88. Amino acid modifications can be independently selected from substitutions, deletions, and insertions.

In some embodiments, the prenyl transferase activity may be provided by an enzyme comprising an amino acid sequence of SEQ ID NO: 89, or a derivative thereof. In some embodiments, the derivative comprises an amino acid sequence having at least 60%, at least 70%, at least 80%, at least 90%, at least 95%, or at least 98% identity to the amino acid sequence of SEQ ID NO: 89. In some embodiments, the derivative comprises an amino acid sequence having from 1 to 20 or from 1 to 10 amino acid modifications with respect to the sequence of SEQ ID NO: 89. Amino acid modifications can be independently selected from substitutions, deletions, and insertions.

In some embodiments, the prenyl transferase activity may be provided by an enzyme comprising an amino acid sequence of SEQ ID NO: 90, or a derivative thereof. In some embodiments, the derivative comprises an amino acid sequence having at least 60%, at least 70%, at least 80%, at least 90%, at least 95%, or at least 98% identity to the amino acid sequence of SEQ ID NO: 90. In some embodiments, the derivative comprises an amino acid sequence having from 1 to 20 or from 1 to 10 amino acid modifications with respect to the sequence of SEQ ID NO: 90. Amino acid modifications can be independently selected from substitutions, deletions, and insertions.

In some embodiments, the prenyl transferase activity may be provided by an enzyme comprising an amino acid sequence of SEQ ID NO: 91, or a derivative thereof. In some embodiments, the derivative comprises an amino acid sequence having at least 60%, at least 70%, at least 80%, at least 90%, at least 95%, or at least 98% identity to the amino acid sequence of SEQ ID NO: 91. In some embodiments, the derivative comprises an amino acid sequence having from 1 to 20 or from 1 to 10 amino acid modifications with respect to the sequence of SEQ ID NO: 91. Amino acid modifications can be independently selected from substitutions, deletions, and insertions.

In some embodiments, the prenyl transferase activity may be provided by an enzyme comprising an amino acid sequence of SEQ ID NO: 93, or a derivative thereof. In some embodiments, the derivative comprises an amino acid sequence having at least 60%, at least 70%, at least 80%, at least 90%, at least 95%, or at least 98% identity to the amino acid sequence of SEQ ID NO: 93. In some embodiments, the derivative comprises an amino acid sequence having from 1 to 20 or from 1 to 10 amino acid modifications with respect to the sequence of SEQ ID NO: 93. Amino acid modifications can be independently selected from substitutions, deletions, and insertions. In various embodiments, the enzymatic pathway further comprises one or more enzymes involved in the production of GPP, such as a GPP synthase (GPPS) and/or enzymes of the methylerythritol phosphate (MEP) and/or mevalonic acid (MVA) pathways. In various embodiments, the enzymatic pathway further comprises one or more enzymes involved in the production of OA, such as an acyl-activating enzyme (AAE), an olivetol synthase (OLS), and/or an olivetolic acid cyclase (OAC). In various embodiments, the enzymatic pathway further comprises one or more enzymes involved in the production of DA, such as an acyl-activating enzyme (AAE), a Divarin synthase (DS) and/or a Divarinic Acid Cyclase (DAC).

In some embodiments, the CBGAS or CBGVAS efficiently directs the flow of precursors into cannabinoids rather than other compounds. For example, in some embodiments, at least 50%, 60%, 70%, 80% or 90% of OA is converted to CBGA. Likewise, at least 50%, 60%, 70%, 80% or 90% of DA may be converted to CBGVA.

In various embodiments, the enzymatic pathway further comprises one or more enzymes that use CBGA as a substrate and catalyze the oxidative cyclization of the monoterpene moiety of CBGA, and such enzyme may be stereoselective. Such enzymes include tetrahydrocannabinolic acid synthase (THCAS), which produces tetrahydrocannabinolic acid (THCA); cannabidiolic acid synthase (CBDAS), which produces cannabidiolic acid (CBDA); and cannabichromenic acid synthase (CBCAS), which produces cannabichromenic acid (CBCA).

In various embodiments, the enzymatic pathway further comprises one or more enzymes that use CBGVA as a substrate and catalyze the oxidative cyclization of the monoterpene moiety of GBGVA, which in some embodiments is stereoselective. Such enzymes include THCAS, which produces tetrahydrocannabivarinic acid (THCVA), CBDAS, which produces cannabidivarinic acid (CBDVA), and CBCAS, which produces cannabichrovarinic acid (CBCVA).

In various embodiments, the enzymatic pathway further comprises enzymes involved in the production of geranyl diphosphate (GPP), such as a GPPS and enzymes in the methylerythritol phosphate (MEP) and/or mevalonic acid (MVA) pathways. GPPS catalyzes a reaction between isopentenyl diphosphate (IPP), and dimethylallyl diphosphate (DMAPP) to form GPP. The GPPS activity may be provided by an enzyme comprising an amino acid sequence selected from SEQ ID NOS: 1 to 25, or a derivative thereof. In some embodiments, the derivative comprises an amino acid sequence having at least 60%, at least 70%, at least 80%, at least 90%, at least 95%, or at least 98% identity to an amino acid sequence selected from SEQ ID NOS: 1 to 25. In some embodiments, the derivative comprises an amino acid sequence having from 1 to 20 or from 1 to 10 amino acid modifications with respect to a sequence selected from SEQ ID NOS: 1 to 25. Amino acid modifications can be independently selected from substitutions, deletions, and insertions.

In some embodiments, the microbial host cell is engineered to express or overexpress one or more enzymes in the MEP and/or MVA pathways to catalyze IPP and DMAPP biosynthesis from glucose or other carbon source. In some embodiments, the microbial host cell is engineered to express or overexpress one or more enzymes of the MEP pathway. In some embodiments, the MEP pathway is increased and balanced with downstream pathways by providing duplicate copies of certain rate-limiting enzymes. The MEP (2-C-methyl-D-erythritol 4-phosphate) pathway, also called the MEP/DOXP (2-C-methyl-D-erythritol 4-phosphate/l-deoxy-D-xylulose 5-phosphate) pathway or the non-mevalonate pathway or the mevalonic acid-independent pathway refers to the pathway that converts glyceraldehyde-3-phosphate and pyruvate to IPP and DMAPP. The pathway typically involves action of the following enzymes: 1-deoxy-D-xylulose-5-phosphate synthase (Dxs), 1-deoxy-D-xylulose-5-phosphate reductoisomerase (IspC), 4-diphosphocytidyl-2-C-methyl-D-erythritol synthase (IspD), 4-diphosphocytidyl-2-C-methyl-D-erythritol kinase (IspE), 2C-methyl-D-erythritol 2,4-cyclodiphosphate synthase (IspF), 1-hydroxy-2-methyl-2-(E)-butenyl 4-diphosphate synthase (IspG), and isopentenyl diphosphate isomerase (IspH). The MEP pathway, and the genes and enzymes that make up the MEP pathway, are described in U.S. Pat. No. 8,512,988, which is hereby incorporated by reference in its entirety. For example, genes that make up the MEP pathway include dxs, ispC, ispD, ispE, ispF, ispG, ispH, idi, and ispA. In some embodiments, the microbial host cell expresses or overexpresses of one or more of dxs, ispC, ispD, ispE, ispF, ispG, ispH, idi, ispA, or modified variants thereof, which results in the increased production of IPP and DMAPP. In some embodiments, GPP is produced at least in part by metabolic flux through an MEP pathway, and wherein the microbial host cell has at least one additional gene copy of one or more of dxs, ispC, ispD, ispE, ispF, ispG, ispH, idi, ispA, or modified variants thereof.

In some embodiments, the microbial host cell is engineered to express or overexpress one or more enzymes of the MVA pathway. The MVA pathway refers to the biosynthetic pathway that converts acetyl-CoA to IPP. The mevalonate pathway typically comprises enzymes that catalyze the following steps: (a) condensing two molecules of acetyl-CoA to acetoacetyl-CoA (e.g., by action of acetoacetyl-CoA thiolase); (b) condensing acetoacetyl-CoA with acetyl-CoA to form hydroxymethylglutaryl-CoenzymeA (HMG-CoA) (e.g., by action of HMG-CoA synthase (HMGS)); (c) converting HMG-CoA to mevalonate (e.g., by action of HMG-CoA reductase (HMGR)); (d) phosphorylating mevalonate to mevalonate 5-phosphate (e.g., by action of mevalonate kinase (MK)); (e) converting mevalonate 5-phosphate to mevalonate 5-pyrophosphate (e.g., by action of phosphomevalonate kinase (PMK)); and (f) converting mevalonate 5-pyrophosphate to isopentenyl pyrophosphate (e.g., by action of mevalonate pyrophosphate decarboxylase (MPD)). The MVA pathway, and the genes and enzymes that make up the MVA pathway, are described in U.S. Pat. No. 7,667,017, which is hereby incorporated by reference in its entirety. In some embodiments, the microbial host cell expresses or overexpresses one or more of acetoacetyl-CoA thiolase, HMGS, HMGR, MK, PMK, and MPD or modified variants thereof, which results in the increased production of IPP and DMAPP. In some embodiments, GPP is produced at least in part by metabolic flux through an MVA pathway, and wherein the microbial host cell has at least one additional gene copy of one or more of acetoacetyl-CoA thiolase, HMGS, HMGR, MK, PMK, MPD, or modified variants thereof.

In some embodiments, the MEP pathway of the microbial host cell is engineered to increase production of IPP and DMAPP from glucose as described in US 2018/0245103 or US 2018/0216137, the contents of which are hereby incorporated by reference in their entireties. For example, in some embodiments the microbial host cell overexpresses MEP pathway enzymes, with balanced expression to push/pull carbon flux to IPP and DMAPP. In some embodiments, the microbial host cell is engineered to increase the availability or activity of Fe—S cluster proteins, so as to support higher activity of IspG and IspH, which are Fe—S enzymes. In some embodiments, the host cell is engineered to overexpress IspG and IspH, so as to provide increased carbon flux to 1-hydroxy-2-methyl-2-(E)-butenyl 4-diphosphate (HMBPP) intermediate, but with balanced expression to prevent accumulation of HMBPP at an amount that reduces cell growth or viability, or at an amount that inhibits MEP pathway flux.

In alternative embodiments, the microbial host cell is not engineered to increase production of GPP from MEP or MVA pathway precursors, but GPP or precursor compound (e.g., a terpene or terpene precursor) is fed to the cells to provide GPP substrate for CBD production.

In various embodiments, the enzymatic pathway further comprises enzymes involved in the production of OA, such as OAC, OLS, or an AAE.

OAC is a polyketide cyclase that can convert olivetol to OA by catalyzing a C2→C7 intramolecular aldol condensation upon which the carboxylate moiety is preserved. The OAC may comprise the amino acid sequence of SEQ ID NO: 52, or a derivative thereof. Alternatively, the OAC activity may be provided by an enzyme comprising an amino acid sequence selected from SEQ ID NOs: 53 to 59, or a derivative thereof. In some embodiments, the derivative comprises an amino acid sequence having at least 60%, at least 70%, at least 80%, at least 90%, at least 95%, or at least 98% identity to an amino acid sequence selected from SEQ ID NOS: 52 to 59. In some embodiments, the derivative comprises an amino acid sequence having from 1 to 20 or from 1 to 10 amino acid modifications with respect to a sequence selected from SEQ ID NOS: 52 to 59. Amino acid modifications can be independently selected from substitutions, deletions, and insertions.

OLS catalyzes the formation of olivetol by the aldol condensation of hexanoyl-CoA with three molecules of malonyl-CoA. The OLS may comprise the amino acid sequence of SEQ ID NO: 49, or a derivative thereof. Alternatively, the OLS activity may be provided by an enzyme comprising an amino acid sequence selected from SEQ ID NOs: 49-51, or a derivative thereof. The OLS enzyme may additionally have, or alternatively have, or be engineered to have, DS activity, and therefore useful for production of C3 cannabinoids. In some embodiments, the derivative comprises an amino acid sequence having at least 60%, at least 70%, at least 80%, at least 90%, at least 95%, or at least 98% identity to an amino acid sequence selected from SEQ ID NOS: 49 to 51. In some embodiments, the derivative comprises an amino acid sequence having from 1 to 20 or from 1 to 10 amino acid modifications with respect to a sequence selected from SEQ ID NOS: 49 to 51. Amino acid modifications can be independently selected from substitutions, deletions, and insertions.

The acyl-activating enzyme (AAE), also called hexanoyl-CoA synthetase, synthesizes hexanoyl-CoA from hexanoate and CoA. Alternatively, the AAE may have or be engineered to have activity for producing Butyric acid instead of Hexanoic acid, and therefore useful for the production of C3 cannabinoids. The AAE may comprise the amino acid sequence of SEQ ID NO: 26, or may be a derivative thereof. Alternatively, the AAE may comprise the amino acid sequence of SEQ ID NO: 27, or a derivative thereof. Alternatively, the AAE activity may be provided by an enzyme comprising an amino acid sequence selected from SEQ ID NOS: 26 to 48, or a derivative thereof. In some embodiments, the derivative comprises an amino acid sequence having at least 60%, at least 70%, at least 80%, at least 90%, at least 95%, or at least 98% identity to an amino acid sequence selected from SEQ ID NOS: 26 to 48. In some embodiments, the derivative comprises an amino acid sequence having from 1 to 20 or from 1 to 10 amino acid modifications with respect to a sequence selected from SEQ ID NOS: 26 to 48. Amino acid modifications can be independently selected from substitutions, deletions, and insertions.

In various embodiments, the enzymatic pathway further comprises enzymes involved in the production of DA, such as a DAC, DS, or an AAE. An enzyme having OAC activity may also have, or be engineered to have, DAC activity, and therefore be useful for production of C3 cannabinoids. Likewise, an enzyme having OLS activity may also have or be engineered to have DS activity; and an enzyme having AAE activity on Hexanoic Acid may also have or be engineered to have AAE activity on Butyric Acid.

In some embodiments, the enzymatic pathway for production of a C5 or C3 cannabinoid comprises an OAC or DAC enzyme comprising an amino acid sequence selected from SEQ ID NOS: 52-59, or a derivative thereof. In some embodiments, the derivative comprises an amino acid sequence having at least 60%, at least 70%, at least 80%, at least 90%, at least 95%, or at least 98% identity to an amino acid sequence selected from SEQ ID NOS: 52 to 59. In some embodiments, the derivative comprises an amino acid sequence having from 1 to 20 or from 1 to 10 amino acid modifications with respect to a sequence selected from SEQ ID NOS: 52 to 59. Amino acid modifications can be independently selected from substitutions, deletions, and insertions.

In some embodiments, the enzymatic pathway for production of a C5 or C3 cannabinoid comprises an OLS or DS enzyme, which may comprise an amino acid sequence selected from SEQ ID NOS: 49 to 51, or a derivative thereof. In some embodiments, the derivative comprises an amino acid sequence having at least 60%, at least 70%, at least 80%, at least 90%, at least 95%, or at least 98% identity to an amino acid sequence selected from SEQ ID NOS: 49 to 51. In some embodiments, the derivative comprises an amino acid sequence having from 1 to 20 or from 1 to 10 amino acid modifications with respect to a sequence selected from SEQ ID NOS: 49 to 51. Amino acid modifications can be independently selected from substitutions, deletions, and insertions.

In various embodiments, the enzymatic pathway further comprises one or more enzymes that convert CBGA or CBGVA into cannabinoid derivatives that are optionally converted by a non-enzymatic process into additional cannabinoid compounds. In various embodiments, one or more nonenzymatic reactions convert THCA to THC, CBDA to CBD, CBCA to CBC, THCVA to THCV, CBDVA to CBDV, and/or CBCVA to CBCV.

In some embodiments, a combination of enzymes are expressed in the pathway to produce a plurality of cannabinoid compounds. Each of the diverse cannabinoid compounds created by these processes has unique and potentially beneficial biological activities.

Enzymes with substrate specificity for CBGA or CBGVA include THCAS, CBDAS, and CBCAS, including derivatives described herein. These enzymes may be derived or engineered from a plant that produces cannabinoids, such as Cannabis sativa.

In some embodiments, the enzymatic pathway comprises a THCAS enzyme comprising the amino acid sequence of SEQ ID NO: 99, or a derivative thereof. Alternatively, the enzymatic pathway comprises a THCAS enzyme comprising an amino acid sequence selected from SEQ ID NOS: 99 to 101, or a derivative thereof. In some embodiments, the derivative comprises an amino acid sequence having at least 60%, at least 70%, at least 80%, at least 90%, at least 95%, or at least 98% identity to an amino acid sequence selected from SEQ ID NOS: 99 to 101. In some embodiments, the derivative comprises an amino acid sequence having from 1 to 20 or from 1 to 10 amino acid modifications with respect to a sequence selected from SEQ ID NOS: 99 to 101. Amino acid modifications can be independently selected from substitutions, deletions, and insertions.

In some embodiments, the enzymatic pathway comprises a CBDAS enzyme comprising the amino acid sequence of SEQ ID NO: 95, or a derivative thereof. Alternatively, the CBDAS enzyme comprises an amino acid sequence selected from SEQ ID NOS: 96 or 97, or a derivative thereof. In some embodiments, the derivative comprises an amino acid sequence having at least 60%, at least 70%, at least 80%, at least 90%, at least 95%, or at least 98% identity to an amino acid sequence selected from SEQ ID NOS: 95 to 97. In some embodiments, the derivative comprises an amino acid sequence having from 1 to 20 or from 1 to 10 amino acid modifications with respect to a sequence selected from SEQ ID NOS: 95 to 97. Amino acid modifications can be independently selected from substitutions, deletions, and insertions.

In some embodiments, the enzymatic pathway comprises a CBCAS enzyme, which may comprise the amino acid sequence of SEQ ID NO: 98, or a derivative thereof. In some embodiments, the derivative comprises an amino acid sequence having at least 60%, at least 70%, at least 80%, at least 90%, at least 95%, or at least 98% identity to the amino acid sequence of SEQ ID NO:98. In some embodiments, the derivative comprises an amino acid sequence having from 1 to 20 or from 1 to 10 amino acid modifications with respect to the sequence of SEQ ID NOS: 98. Amino acid modifications can be independently selected from substitutions, deletions, and insertions.

The term “or a derivative thereof” indicates some degree of similarity between the derivative and a “parent” enzyme having the recited sequence. A derivative may have at least 40%, 50%, 60%, 70%, 75%, 80%, 85%, 90%, 95%, 98%, or 99% sequence identity with a parent enzyme. A derivative may also share structural similarity with a parent enzyme, such as similarity in secondary, tertiary, or quaternary structure. In various embodiments, a derivative and parent enzyme have similar substrate and/or cofactor binding sites, active sites, or reaction mechanisms.

The identity of amino acid sequences, i.e. the percentage of sequence identity, can be determined via sequence alignments. Such alignments can be carried out with several art-known algorithms, such as with the mathematical algorithm of Karlin and Altschul (Karlin & Altschul (1993) Proc. Natl. Acad. Sci. USA 90: 5873-5877), with hmmalign (HMMER package, http://hmmer.wustl.edu/) or with the CLUSTAL algorithm (Thompson, J. D., Higgins, D. G. & Gibson, T. J. (1994) Nucleic Acids Res. 22, 4673-80). The grade of sequence identity (sequence matching) may be calculated using e.g. BLAST, BLAT or BlastZ (or BlastX). A similar algorithm is incorporated into the BLASTN and BLASTP programs of Altschul et al (1990) J. Mol. Biol. 215: 403-410. BLAST protein searches may be performed with the BLASTP program, score=50, word length=3. To obtain gapped alignments for comparative purposes, Gapped BLAST is utilized as described in Altschul et al (1997) Nucleic Acids Res. 25: 3389-3402. When utilizing BLAST and Gapped BLAST programs, the default parameters of the respective programs are used. Sequence matching analysis may be supplemented by established homology mapping techniques like Shuffle-LAGAN (Brudno M., Bioinformatics 2003b, 19 Suppl 1:154-162) or Markov random fields.

In various embodiments, two or more heterologous enzymes are expressed together in an operon, or are expressed individually. The enzymes may be expressed from extrachromosomal elements such as plasmids, or bacterial artificial chromosomes, or may be chromosomally integrated.

The amounts of various cannabinoids and cannabinoid precursors can be measured in a recombinant host cell to identify rate limiting steps in the biosynthetic pathway. Once a rate-limiting step has been identified, expression or activity of the limiting enzyme can be increased by various methods known in the art, such as codon optimization, use of a stronger promotor, expressing multiple copies of the corresponding gene, and constructing variants with increase stability and/or activity.

In some embodiments, one or more cannabinoids produced by a recombinant host cell are partially or completely exported to the culture medium. In other embodiments, one or more cannabinoids produced by a recombinant host cell are retained within the recombinant cell. Cannabinoids can be recovered from the culture medium or from the recombinant host cell.

In various embodiments, the microbe cell is a bacterium, and may be of a genus selected from Escherichia, Bacillus, Corynebacterium, Rhodobacter, Zymomonas, Vibrio, Pseudomonas, Agrobacterium, Brevibacterium, and Paracoccus. In some embodiments, the bacterium is a species selected from Escherichia coli, Bacillus subtilis, Corynebacterium glutamicum, Rhodobacter capsulatus, Rhodobacter sphaeroides, Zymomonas mobilis, Vibrio natriegens, or Pseudomonas putida. In some embodiments, the bacterium is E. coli. In various embodiments, the microbial cell is a yeast cell, which is a species of Saccharomyces, Pichia, or Yarrowia. For example, the microbial cell may be a species selected from Saccharomyces cerevisiae, Pichia pastoris, and Yarrowia lipolytica.

In various embodiments, a recombinant host cell incorporates modifications that increase the pool of acyl-CoA precursors to enable high-titer production of OA and DA pathway intermediates. In these or other embodiments, the host cell is modified for enhanced GPP production. In some embodiments, a recombinant E. coli cell overexpresses one or more enzymes of the MEP pathway. The E. coli may have engineered expression of MEP pathway enzymes and other modifications as described in US 2018/0245103 or US 2018/0216137, the contents of which are hereby incorporated by reference in their entireties.

In some embodiments, the microbial host cell is a species of Saccharomyces, Pichia, or Yarrowia, including, but not limited to, Saccharomyces cerevisiae, Pichia pastoris, and Yarrowia lipolytica.

In some embodiments, the host cell is the oleaginous yeast Yarrowia lipolytica, which can utilize a wide variety of carbon sources and has the potential for high flux through key cannabinoid precursors, acetyl-CoA and malonyl-CoA. PCT/US2017/022252, which is hereby incorporated by reference in its entirety, presents various methods for increasing the biosynthesis of polyketides such as OA and DA in yeast by metabolic engineering. Polyketide synthesis is enhanced by reducing or eliminating the expression of certain genes, and by overexpressing other genes.

In yeast species such as Y. lipolytica, coordinated overexpression of pyruvate dehydrogenase complex components PDA1, PDE2, PDE3, and PDB1 with ACC1, the enzyme that converts acetyl-CoA to malonyl-CoA, is useful to increase polyketide synthesis. Enhanced expression of pyruvate bypass pathway enzymes further increase polyketide synthesis. These enzymes convert pyruvate to acetaldehyde through pyruvate decarboxylase (PDC1, PDC2), and then to acetate through acetylaldehde dehydrogenase (ALD2, ALD3, ALD5), and finally to acetyl-CoA via acetyl-CoA synthase (ACS1). For example, polyketide synthesis can be increased in some embodiments upon overexpression of various combinations of ACS1, ALD2, ALD3, ALD5, PDC1, PDC2 and ACC1. Additionally, genetic modifications such as overproduction of peroxisomal matrix protein 10 (PEX10), multifunctional β oxidation protein (MFE1), primary oleate regulator (POR1) or phosphatidate phosphatase (PAH) can increase β-oxidation of fatty acids and thereby increase the availability of acetyl-CoA and malonyl-CoA.

In some embodiments, a recombinant yeast (e.g., Y. lipolytica) host cell is engineered to incorporate modifications that increase the pool of acyl-CoA precursors to enable high-titer production of OA or DA pathway intermediates. In various embodiments, the recombinant yeast cell is modified for enhanced GPP production, which can be through overexpression of one or more enzymes of the MVA pathway. In alternative embodiments, the yeast cell does not overexpress enzymes of the MVA pathway, or is not engineered for increased production of MVA pathway products, and instead the cell may be fed GPP or terpene or terpene precursor compounds to support cannabinoid biosynthesis. In some embodiments, the cell produces GPP from IPP and/or DMAPP. In embodiments, the microbial cell expresses one or more enzymes for converting fed isoprenol and/or prenol to isopentenyl pyrophosphate (IPP) and/or dimethylallyl pyrophosphate (DMAPP), and, in some embodiments, the one or more enzymes are optionally kinases.

In some embodiments, recombinant host cells can produce cannabinoids from sugar (e.g., glucose) and other components present in growth media. In other embodiments, cannabinoids are produced by bioconversion from precursors, such as, olivetol, OA, divarin, DA, hexanoic acid, butyric acid, hexanoyl-CoA, butyryl-CoA and GPP precursor, which are fed to recombinant cells. In various embodiments, cannabinoids are produced from one or more alternative carbon sources including, for example, C1, C2, C3, C4, C5, and/or C6 carbon substrates, glycerol, xylose, fructose, mannose, ribose, sucrose, lignocellulosic biomass, ethanol, acetate, beet pulp, black liquor, corn starch, or switchgrass.

In some embodiments, the recombinant host cell expresses enzymes having CBGAS and CBDAS activity, and thus produces CBDA, which can be converted to CBD.

In some embodiments, the recombinant host cell expresses enzymes having CBGAS and CBDAS activity, and produces CBDA and/or CBD when fed with media comprising sugar such as glucose, or other carbon C1 to C6 carbon substrates. Such recombinant host cells may further express enzymes having GPPS, OAC, OLS, and/or AAE activity. In some embodiments, the recombinant host cell expressing CBGAS and CBDAS enzymes produces CBDA and/or CBD when fed with olivetol or OA. In some embodiments, CBDA recovered from a recombinant host cell is converted to CBD by exposure to heat and/or UV light.

In some embodiments, a recombinant host cell expresses enzymes having CBGAS and THCAS activity, the host cell producing THCA, which can be converted to THC. In some embodiments, the recombinant host cell expressing enzymes having CBGAS and THCAS activity produces THCA, which can convert to THC, when fed with media comprising sugar such as glucose or other C1 to C6 carbon substrates. In such embodiments, the recombinant host cell further expresses GPPS, OLS and/or OAC enzymes. In some embodiments the recombinant host cell expresses enzymes having CBGAS and THCAS activity, the host cell producing THCA, which can convert to THC, when fed with olivetol or OA. In some embodiments, THCA recovered from a recombinant host cell is converted to THC by exposure to heat and/or UV light.

In some embodiments, a recombinant host cell expresses enzymes having CBGAS and CBCAS activity, the host cell producing CBCA, which can be converted to CBC. In some embodiments, the recombinant host cell expressing enzymes having CBGAS and CBCAS activity produces CBCA, which can convert to CBC, when fed with media comprising sugar such as glucose or other C1 to C6 carbon substrates. In such embodiments, the recombinant host cell further expresses GPPS, OLS and/or OAC enzymes. In some embodiments the recombinant host cell expresses enzymes having CBGAS and CBCAS activity, the host cell producing CBCA, which can convert to CBC, when fed with olivetol or OA. In some embodiments, CBCA recovered from a recombinant host cell is converted to CBC by exposure to heat and/or UV light.

In some embodiments, a recombinant host cell expresses enzymes having CBGVAS and THCAS activity, the host cell producing THCVA, which can be converted to THCV. In some embodiments, the recombinant host cell expressing enzymes having CBGVAS and THCAS activity produces THCVA, which can convert to THCV, when fed with media comprising sugar such as glucose or other C1 to C6 carbon substrates. In such embodiments, the recombinant host cell further expresses GPPS, DS and/or DAC enzymes. In some embodiments the recombinant host cell expresses enzymes having CBGVAS and THCAS activity, the host cell producing THCVA, which can convert to THCV, when fed with divarin or DA. In some embodiments, THCVA recovered from a recombinant host cell is converted to THCV by exposure to heat and/or UV light.

In some embodiments, a recombinant host cell expresses enzymes having CBGVAS and CBDAS activity, the host cell producing CBDVA, which can be converted to CBDV. In some embodiments, the recombinant host cell expressing enzymes having CBGVAS and CBDAS activity produces CBDVA, which can convert to CBDV, when fed with media comprising sugar such as glucose or other C1 to C6 carbon substrates. In such embodiments, the recombinant host cell further expresses GPPS, DS and/or DAC enzymes. In some embodiments the recombinant host cell expresses enzymes having CBGVAS and CBDAS activity, the host cell producing CBDVA, which can convert to CBDV, when fed with divarin or DA. In some embodiments, CBDVA recovered from a recombinant host cell is converted to CBDV by exposure to heat and/or UV light.

In some embodiments, a recombinant host cell expresses enzymes having CBGVAS and CBCAS activity, the host cell producing CBCVA, which can be converted to CBCV. In some embodiments, the recombinant host cell expressing enzymes having CBGVAS and CBCAS activity produces CBCVA, which can convert to CBCV, when fed with media comprising sugar such as glucose or other C1 to C6 carbon substrates. In such embodiments, the recombinant host cell further expresses GPPS, DS and/or DAC enzymes. In some embodiments the recombinant host cell expresses enzymes having CBGVAS and CBCAS activity, the host cell producing CBCVA, which can convert to CBCV when fed with divarin or DA. In some embodiments, CBCVA recovered from a recombinant host cell is converted to CBCV by exposure to heat and/or UV light.

In various embodiments, the host cell is cultured at a temperature between 22° C. and 37° C. While commercial biosynthesis in host cells such as E. coli can be limited by the temperature at which overexpressed and/or foreign enzymes (e.g., enzymes derived from plants) are stable, recombinant enzymes (including the terpenoid synthase) may be engineered to allow for cultures to be maintained at higher temperatures, resulting in higher yields and higher overall productivity. In some embodiments, the host cell (bacterial or yeast host cell) is cultured at about 22° C. or greater, about 23° C. or greater, about 24° C. or greater, about 25° C. or greater, about 26° C. or greater, about 27° C. or greater, about 28° C. or greater, about 29° C. or greater, about 30° C. or greater, about 31° C. or greater, about 32° C. or greater, about 33° C. or greater, about 34° C. or greater, about 35° C. or greater, about 36° C. or greater, or about 37° C.

Cannabinoids can be extracted from media and/or whole cells, and recovered. In some embodiments, the cannabinoids are recovered and optionally enriched by fractionation (e.g. fractional distillation). The product can be recovered by any suitable process, including partitioning the desired product into an organic phase. Various methods of cannabinoid preparation are known in the art, such as centrifugal partition chromatography. The production of the desired product can be determined and/or quantified, for example, by gas chromatography (e.g., GC-MS) or high pressure liquid chromatography (HPLC-MS).

The desired product can be produced in batch or continuous bioreactor systems. Production of product, recovery, and/or analysis of the product can be done as described in US 2012/0246767, which is hereby incorporated by reference in its entirety. For example, in some embodiments, oxidized oil is extracted from aqueous reaction medium, which may be done by partitioning into an organic phase, followed by fractional distillation. Cannabinoid components of fractions may be measured quantitatively by GC/MS or HPLC/MS, followed by blending of the fractions.

In some embodiments, the microbial host cells and methods disclosed herein are suitable for commercial production of one or more cannabinoids, that is, the microbial host cells and methods are productive at commercial scale. In some embodiments, the size of the culture is at least about 100 L, at least about 200 L, at least about 500 L, at least about 1,000 L, at least about 10,000 L, at least about 100,000 L, or at least about 1,000,000 L. In some embodiment, the culturing may be conducted in batch culture, continuous culture, or semi-continuous culture.

In some aspects, the present disclosure provides methods for making a product comprising one or more cannabinoids. In various aspects, the product is a pharmaceutical composition, a dietary supplement or a baked good. A cannabinoid of the present invention can be mixed with one or more excipients to form a pharmaceutical product, which may be a pill, a capsule, a mouth spray, or an oral solution.

As used in this specification and the appended claims, the singular forms “a”, “an” and “the” include plural referents unless the content clearly dictates otherwise. For example, reference to “a cell” includes a combination of two or more cells, and the like.

### EXAMPLES

### Example 1: Production of Cannabigerolic Acid by Prenyl Transferases

Several candidate prenyltransferases (Table 1) were screened using liquid chromatography (LC) mass spectrometry (MS/MS) for their ability to generate cannabigerolic acid (CBGA).

Olivetolic acid (OA) and geranyl pyrophosphate (GPP) (both substrates) were mixed with each candidate prenyltransferase and reactions were performed under conditions suitable for production of CBGA. Products generated from the reaction of each candidate prenyl transferase were identified by multiple reaction monitoring and their retention times were compared to the authentic CBGA standard. The results obtained for each candidate prenyltransferase is shown in Table 1 below.

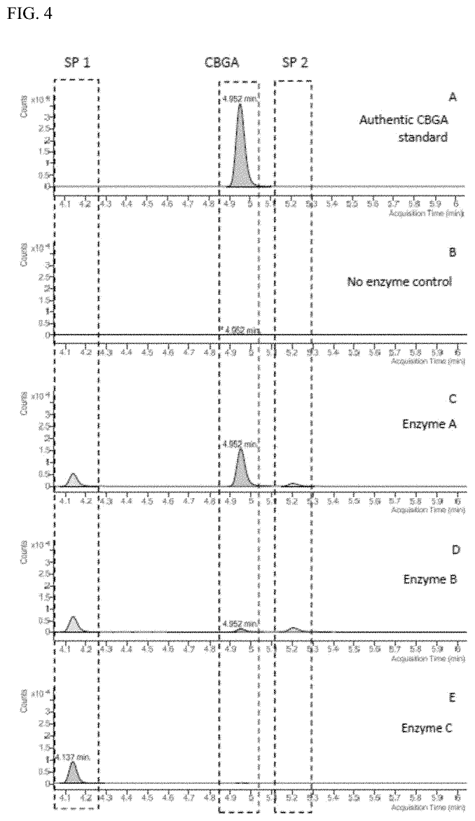

Each panel in FIG. 4 shows the retention times on the X-axis and ion counts (m/z 361.0>219.0) on the Y-axis. SP (1 or 2) represents the side product obtained from the reaction. FIG. 4A shows the authentic CBGA standard having a retention time of 4.952 min. FIG. 4B shows products obtained from a control where no enzyme was added to the reaction mix. No CBGA was produced in the control. FIG. 4C shows the reaction products obtained from Enzyme A; CBGA was produced as shown in the figure having a retention time of 4.952 min. FIG. 4D shows the reaction products obtained from Enzyme B and FIG. 4E shows the reaction products obtained from Enzyme C.

TABLE 1
A List of Aromatic Prenyltransferase Candidates
and Their Cannabigerolic Acid (CBGA) Activity.

| Enzyme (SEQ ID NO) | CBGA Activity |
|---|---|
| 1 (SEQ ID NO: 63) | Yes |
| 2 (SEQ ID NO: 64) | No |
| 3 (SEQ ID NO: 65) | No |
| 4 (SEQ ID NO: 66) | No |
| 5 (SEQ ID NO: 67) | No |
| 6 (SEQ ID NO: 68) | No |
| 7 (SEQ ID NO: 69) | No |
| 8 (SEQ ID NO: 70) | No |
| 9 (SEQ ID NO: 71) | No |
| 10 (SEQ ID NO: 72) | No |
| 11 (SEQ ID NO: 73) | No |
| 12 (SEQ ID NO: 74) | Yes |
| 13 (SEQ ID NO: 75) | No |
| 14 (SEQ ID NO: 76) | No |
| 15 (SEQ ID NO: 77) | Yes |
| 16 (SEQ ID NO: 78) | No |
| 17 (SEQ ID NO: 79) | No |
| 18 (SEQ ID NO: 80) | No |
| 19 (SEQ ID NO: 81) | No |
| 20 (SEQ ID NO: 82) | No |
| 21 (SEQ ID NO: 83) | No |
| 22 (SEQ ID NO: 60) | No |
| 23 (SEQ ID NO: 61) | No |
| 24 (SEQ ID NO: 62) | No |
| 25 (SEQ ID NO: 84) | Yes |
| 26 (SEQ ID NO: 85) | Yes |
| 27 (SEQ ID NO: 86) | Yes |
| 28 (SEQ ID NO: 87) | Yes |
| 29 (SEQ ID NO: 88) | Yes |
| 30 (SEQ ID NO: 89) | Yes |
| 31 (SEQ ID NO: 90) | Yes |
| 32 (SEQ ID NO: 91) | Yes |
| 33 (SEQ ID NO: 92) | No |
| 34 (SEQ ID NO: 93) | Yes |
| 35 (SEQ ID NO: 94) | No |
| 36 (SEQ ID NO: 84 comprising | Yes |
| a single mutation: G286S) | |

SEQUENCES
GPPS
(Gentiana rigescens)
SEQ ID NO: 1
MALIYSTPSWVQAHTISIYHGNGSSFFPCY
LSKNKAPVFLSNPCKKPNLGRSPLSICAIL
TKEESKIKKAHDFSFNFKDYMLEKADSVNK
ALEQAVSIREPLKIHESMRYSLLAGGKRVR
PMLCIAACELFGGDESVAMPSACAVEMIHT
MSLMHDDLPCMDNDDLRRGKPTNHKVYGED
VAVLAGDALLAFAFEHIATSTKGVTSERIV
RVIGELAKCIGSEGLVAGQIVDVCSEGISD
VGLQHLEFIHIHKTAALLEGSVAMGAILGG
ADDEEVSKLRKFARGIGLLFQVVDDILDVT
KSSKELGKTAAKDLVADKVTYPKLIGIDKS
REFAEKLNREAQDQLAGFDSEKAAPLIALA
NYIAYRDN
(Swertia mussotii)
SEQ ID NO: 2
MSLVNSTATSWLQAHTISNYYGGNGSNLSP
YYLCHTFKNKLGPPISQKESTFRYSSFSIC
AILTKEESKIKKAHDFSSFNFEDYMIEKAN
SVNKALESAVSIREPLKIHESMRYSLLAGG
KRIRPMLCIAACELFGGDESIAMPSACAVE
MIHTMSLMHDDLPCMDNDDLRRGKPTNHKV
FGEDVAVLAGDALLAFAFEHIATSTKGVSS
DRIVRVIGELARFVGSEGLVAGQIVDVCSE
GKSDVGLKHLEFIHIHKTAALLEGSVALGA
ILGGANDEQVLKLKKFARGIGLLFQVVDDI
LDVTKSSKELGKTAGKDLVADKVTYPKLIG
IEKSREFADKLNREAQEQLSGFDPEKAAPL
IALANYIAYRDN
(Camptotheca acuminate)
SEQ ID NO: 3
MLFYRGLSRISRTSLNHGWWLLSFRNEQQL
VPSNNFHYPRYTAEKVLGCRETYSWASHTF
HGVGHQIHHQSCTIDEEQLDPFSLVADELS
VLANRLRSMVVAEVPKLASAAEYLFKMGVE
GKRFRPTVLLLMATALNVPIPGPAPDRSVD
SLSMELRTRQQCIAEITEMIHVASLLHDDV
LDDADTRRGIGSLNFIMGNKLAVLGGDFLL
SRACVALASLKNTEVVSLLATVVEHLVTGE
TMQMTTSSEQRCSMEYYLQKTYYKTASLIS
NSCKAVALLAGQTAEVSLLAYEYGKNLGLA
YQLIDDVLDFIGTSTSLGKGSLSDIRHGIV
TAPILYAIEEFPQLRAVVDEGFDKPANVDL
ALQYLGRSCGIQRTRELATKHANLASAAID
SLPESNDEDVQKSRRALVGLTHRVITRTK
(Arabidopsis thaliana)
SEQ ID NO: 4
MLFTRSVARISSKFLRNRSFYGSSQSLASH
RFAIIPDQGHSCSDSPHKGYVCRTTYSLKS
PVFGGFSHQLYHQSSSLVEEELDPFSLVAD
ELSLLSNKLREMVLAEVPKLASAAEYFFKR
GVQGKQFRSTILLLMATALNVRVPEALIGE
STDIVTSELRVRQRGIAEITEMIHVASLLH
DDVLDDADTRRGVGSLNVVMGNKMSVLAGD
FLLSRACGALAALKNTEVVALLATAVEHLV
TGETMEITSSTEQRYSMDYYMQKTYYKTAS
LISNSCKAVAVLTGQTAEVAVLAFEYGRNL
GLAFQLIDDILDFTGTSASLGKGSLSDIRH
GVITAPILFAMEEFPQLREVVDQVEKDPRN
VDIALEYLGKSKGIQRARELAMEHANLAAA
AIGSLPETDNEDVKRSRRALIDLTHRVITR
NK
(Arabidopsis thaliana)
SEQ ID NO: 5
MVLAEVPKLASAAEYFFKRGVQGKQFRSTI
LLLMATALNVRVPEALIGESTDIVTSELRV
RQRGIAEITEMIHVASLLHDDVLDDADTRR
GVGSLNVVMGNKMSVLAGDFLLSRACGALA
ALKNTEVVALLATAVEHLVTGETMEITSST
EQRYSMDYYMQKTYYKTASLISNSCKAVAV
LTGQTAEVAVLAFEYGRNLGLAFQLIDDIL
DFTGTSASLGKGSLSDIRHGVITAPILFAM
EEFPQLREVVDQVEKDPRNVDIALEYLGKS
KGIQRARELAMEHANLAAAAIGSLPETDNE
DVKRSRRALIDLTHRVITRNK
(Glycine max)
SEQ ID NO: 6
MLGALLLNANFKIHFSLISCQARVPLPVKP
APLRMPSPHYPHWASLQADIEAHLKQTIPL
KEPLEVFEPMLHLAFSAPRTTVPALCLAAC
ELVGGHRQQAMAAASALLLNLANAHAHEHL
TDGPMYGPNIELLTGDGIVPFGFELLARPD
GPASASPERVLRVMIEISRAVGSVGLQDAQ
YVKKTLWDGGEEVQNVESMQRFVLEKRDGG
LHACGAASGAILGGGSEDQIERLRNFGFHV
GMMRGMLQMGFMEKHVQEERHLALKELQFF
MDRDVHVISSFIY
(Helianthus annuus)
SEQ ID NO: 7
MSIYRAISRITRTASSYNRCRWFYSSAPHQ
QLSPYSGFRSSEQVLGCRVISPWFSRSFRS
GGPQPQYEDDQEDPFSLVADELSIVANRLR
SMVVAEVPKLASAAEYFFKMGVEGKRFRPT
VILLMATALNNQISKPPSEGVVDMLSTEFR
TRLQSIAEITEMIHVASLLHDDVLDDADTR
RGIGSLNFVMGNKISVLAGDFLLSRACITL
ASLKNTEVVSLIATAVEHLVTGETMQMSSS
AEQRSSMDYYLQKTYYKTASLISNSCKSIA
LLTGQTAEVAMLAYEYGKNLGLAFQLIDDV
LDFTGTSSSLGKGSLSDIRHGIVTAPLLYA
MEEFFELRSVVDRGLDNPANVDLALEYLGK
SHGIQRTRELAAKHASLASAAIDSFPENDD
EDVQRSRRALIELTHRVINRTK
(Withania somnifera)
SEQ ID NO: 8
MIFSRVLSQISRNRFSRCRWLFSLPPHQQL
HHSNNIYASQKVLGCRVIHSWVSNALSGIG
QQIHHQTSAVAEEQVDPFSLVADELSLLTN
RLRSMVVAEVPKLASAAEYFFKMGVEGKRF
RPTVLLLMATALNVQIPRSAPHVDVDSLSG
DLRTRQQCIAEITEMIHVASLLHDDVLDDA
ETRRGIGSLNYVMGNKLAVLAGDFLLSRAC
VALASLKNTEVVSLLATVVEHLVTGETMQM
TTSSDERCSMEYYMQKTYYKTASLISNSCK
AIALLAGHTAEVSVLAFDYGKNLGLAFQLI
DDVLDFTGTSATLGKGSLSDIRHGIVTAPI
LYAMEEFPQLRTLVDRGFDDPVNVEIALDY
LGKSRGIQRTRELARKHASLASAAIDSLPE
SHDEEVQRSRRALVELTHRVITRTK
(Selaginella moellendorffii)
SEQ ID NO: 9
MAQLGRRLRDMVAAEVPKLASAAEYFFKLG
VEGKRFRPMVLLLMSSSLTMVLPSAAAATS
DEKNWRHHKLAEITEMIHVASLLHDDVLDH
ADTRRGIASLNFIMGNKLAVLAGDFLLARA
AFSLSTLQNDEVVGLMSKVLEHLVAGEVMQ
WTVDAEKSSSMDYYLQKTFYKTASLIANSC
KCIAILAGHPKEVAALAFDYGRHLGLAYQL
VDDLLDFIGTKASLGKPALSDLREGIATAP
VLYALEEHPALQELIDRKFKDPGDVDSALK
MVLASSGIRKTKELAREHASKAADAVAGFP
PTTSEKASLCRRALTELTEQVITRSNRGRM
CCEAVNLSARFN
(Paeonia lactiflora)
SEQ ID NO: 10
MLYSRGFSRIPRNSLIRCCKWFLSSQQYHQ
QSFLSIKFQPPTDHTQKVLGCREIYSRGLL
ALHGIQHQSYHGGSSVIEERLDPFSLVADE
LSVIANRLRAMVVAKVPKLGSAAEYFFKIG
VEGKRFRPTILLLMATALNVSIPGRAHAVL
GDTLATELRTRQQCIAEITEMIHVASLLHD
DVLDDADTRRGISSLNSVVGNKVAVLAGDF
LLSRACVALASLRNTDVVILLATVVEHLVT
GETMQMITTSEQRCSMDYYMEKTYYKTASL
ISNSCKAIALLAGQTAEVAMLAFEYGKNLG
LAFQLIDDVLDFTGTSASLGKGSLSDIRRG
IVTAPILFAVEEFPQLRALVDRGFHDPKDV
DIALDYLGKSCGIQKTRELATKHANLAAAA
IDSLPESDDEEVVKSRRALVDLTQRVITRT
K
(Catharanthus roseus)
SEQ ID NO: 11
MLFSRGLYRIARTSLNRSRLLYPLQSQSPE
LLQSFQFRSPIGSSQKVSGFRVIYSWVSSP
LANVGQQVQRQSNSVAEEPLDPFSLVADEL
SILANRLRSMVVAEVPKLASAAEYFFKLGV
EGKRFRPTVLLLMATAIDAPISRIPPDTSL
DTLSTELRLRQQTIAEITKMIHVASLLHDD
VLDDAETRRGIGSLNFVMGNKLAVLAGDFL
LSRACVALASLKNTEVVSLLATVVEHLVTC
ETMQMITTSDQRCSMEYYMQKTYYMTASLI
SNSCKAIALLAGQTSEVAMLAYEYGKNLGL
AFQLIDDVLDFIGTSASLGKGSLSDIRHGI
VTAPILFAIEEFPELRAVVDEGFENPYNVE
LALHYLGKSRGIQRTRELAIKHANLASDAI
DSLPVTDDEHVLRSRRALVELTQRVITRRE
(Nannochloropsis gaditana)
SEQ ID NO: 12
MPAPRKVGLRRLRGLVQSCSTGFRGGVQPS
LISSRTAISYVNRAVDHIYYSHASIGSTTN
IVHRSIRSGWAKTAADASIDVIVNAVTRPE
IDEPTVKVAEPRRAIIKADQAGELEEDLAL
DLQRKPRLDLLAGWAGAARGVDPFKIVESD
MRSLSAGIKSLLGSDHPVLEACAKYFFELD
GGKKIRPTMVLLISRAVAAHAPAQGVNGSR
AFTSTSESSTPLPSQKRLAEITEMIHTASL
FHDDVIDEADERRGVPSINKIYGNKMAILA
GDFLLARASVSLARLRNIEVVELLSTVIEH
LVKGEVMQSRPQALVDGSGTGENGQAALEY
YLHKNFYKTGSLMANSCRAAVLLAGGGDAL
QNQAFAYGRHVGLAFQLVDDVLDFEQTSET
LGKPALNDLRQGLATAPVLLAARTFPDEVC
DMVKRKFASEGDVERVREMAFFSIAMTSPR
PRYNSSYLGTLL
(Salvia miltiorrhiza)
SEQ ID NO: 13
MISVRGLARLARSGYARRRWVYSSLGCSGS
APLQLEHSSHFRNPIQSSREVLGCRVIYSW
VSNAISTVGQQVHLQSSSAVEEQLDPFSLV
ADELSILADRLRSMVVAEVPKLASAAEYFF
KFGVEGKRFRPTVLLLMATALDLPIARQTS
EVAVNTLSTELRTRQQCVAEITEMIHVASL
LHDDVLDDADTRRGIGSLNYVMGNKLAVLA
GDFLLSRACVALASLKNTEVVTLIAQVVEH
LVTGETMQMITTSEQRCSMEYYMEKTYYKT
ASLICNSCKSIALIAGQTAEVSNLAYEYGE
NLGLAFQIIDDVLDFTGTSASLGKGSLSDI
RHGIVTAPILFAIEEYPELRKIVDQGFEKS
SNVDRALEILSKSSGIQRARELAAKHARLA
SAAIDALPENEDEVVQRSMRALVELTHIVI
TRTK
(Vitis vinifera)
SEQ ID NO: 14
MVVAEVPKLASAAEYFFKMGVEGKRXRPTV
LLLMATALNVPLPRPALAEVPETLSTELRT
RQQCIAEITEMIHVASLLHDDVLDDAETRR
GIGSLNIMMGNKVAVLAGDFLLSRACVALA
SLKNTEVVSLLATVVEHLVTGETMQMTSTS
EQRVSMEYYLQKTYYKTASLISNSCKAIAL
LAGQTAEVSMLAFEYGKNLGLAFQLIDDXL
DFTGTSASLGKGSLSDIRHGIITAPILFAI
EEFPQLDAVVKRGLDNPADIDLALDYLGRS
RGIQRTRELAMKHANLAAEAIDSLPESGDE
DVLRSRRALIDLTHRVITRTK
(Ips pini)
SEQ ID NO: 15
MFKLAQRLPKSVSSLGSQLSKNAPNQLAAA
TTSQLINTPGIRHKSRSSAVPSSLSKSMYD
HNEEMKAAMKYMDEIYPEVMGQIEKVPQYE
EIKPILVRLREAIDYTVPYGKRFKGVHIVS
HFKLLADPKFITPENVKLSGVLGWCAEIIQ
AYFCMLDDIMDDSDTRRGKFTWYKLPGIGL
NAVTDVCLMEMFTFELLKRYFPKHPSYADI
HEILRNLLFLTHMGQGYDFTFIDPVTRKIN
FNDFTEENYTKLCRYKIIFSTFHNTLELTS
AMANVYDPKKIKQLDPVLMRIGMMHQSQND
FKDLYRDQGEVLKQAEKSVLGTDIKTGQLT
WFAQKALSICNDRQRKIIMDNYGKEDNKNS
EAVREVYEELDLKGKFMEFEEESFEWLKKE
IPKINNGIPHKVFQDYTYGVFKRRPE
(Quercus robur)
SEQ ID NO: 16
MLFSRISRIRRPGSNGFRWFLSHKTHLQFL
NPPAYSYSSTHKVLGCREIFSWGLPALHGF
RHNIHHQSSSIVEEQNDPFSLVADELSMVA
NRLRSMVVTEVPKLASAAEYFFKMGVEGKR
FRPTVLLLMATAMNISILEPSLRGPGDALT
TELRARQQRIAEITEMIHVASLLHDDVLDD
ADTRRGIGSLNFVMGNKLAVLAGDFLLSRA
CVALASLKNTEVVSLLAKVVEHLVTGETMQ
MTTTCEQRCSMEYYMQKTYYKTASLISNSC
KAIALLGGQTSEVAMLAYEYGKNLGLAYQL
IDDVLDFTGTSASLGKGSLSDIRHGIITAP
ILFAMEEFPQLREVVDRGFDDPANVDVALD
YLGKSRGIQRARELAKKHANIAAEAIDSLP
ESNDEDVRKSRRALLDLTERVITRTK
(Citrus sinensis)
SEQ ID NO: 17
MVIAEVPKLASAAEYFFKMGVEGKRFRPTV
LLLMATALNVRVPEPLHDGVEDASATELRT
RQQCIAEITEMIHVASLLHDDVLDDADTRR
GIGSLNFVMGNKLAVLAGDFLLSRACVALA
SLKNTEVVILLATVVEHLVTGETMQMTTSS
DQRCSMDYYMQKTYYKTASLISNSCKAIAL
LAGQTAEVAILAFDYGKNLGLAYQLIDDVL
DFTGTSASLGKGSLSDIRHGIITAPILFAM
EEFPQLRTVVEQGFEDSSNVDIALEYLGKS
RGIQKTRELAVKHANLAAAAIDSLPENNDE
DVTKSRRALLDLTHRVITRNK
(Cannabis sativa)
SEQ ID NO: 18
MHRVSLLCSFSQNQKASIFVKTKKMSTVNL
TWVQTCSMFNQGGRSRSLSTFNLNLYHPLK
KTPFSIQTPKQKRPTSPFSSISAVLTEQEA
VKEGDEEKSIFNFKSYMVQKANSVNQALDS
AVLLRDPIMIHESMRYSLLAGGKRVRPMLC
LSACELVGGKESVAMPAACAVEMIHTMSLI
HDDLPCMDNDDLRRGKPTNHKVFGEDVAVL
AGDALLAFAFEHMAVSTVGVPAAKIVRAIG
ELAKSIGSEGLVAGQVVDIDSEGLANVGLE
QLEFIHLHKTGALLEASVVLGAILGGGIDE
EVEKLRSFARCIGLLFQVVDDILDVTKSSQ
ELGKTAGKDLVADKVTYPRLMGIDKSREFA
EQLNTEAKQHLSGFDPIKAAPLIALANYIA
YRQN
(Morus alba)
SEQ ID NO: 19
MSCVNLSTWVQTCSLFNQAGGRSRLSSSSA
LNNLFHPLKNNFPVPLSSIPKRHRPSPSSS
LSTVSAVLTQQETETVTEVLEEEKAPFNFK
AYMIQKANSVNQALDDAVSLREPQTIHEAM
RYSLLAGGKRVRPVLCLTACELVGGDESVA
MPAALAVEMIHTMSLIHDDLPCMDNDDLRR
GKPTNHKVFGEDVAVLAGDALLAFAFEHIA
VSTAGVTPSRIVRAIGELAKSIGTEGLVAG
QVVDIDSEGSDDAGLEKLEFIHIHKTAALL
EASVVLGAILGGGTDDEVEKLRSFARCIGL
LFQVVDDILDVTKSSQELGKTAGKDLVADK
VTYPKLIGIEKSKEFAAKLNKEAQEQLSGF
DPHKAAPLIALANYIANRQN
(Alcanivorax borkumensis SK2)
SEQ ID NO: 20
MSSKATREFAALNQLTDTAKARLEQALDHY
LPAHSAASRLSHAMRYAALSGGKRIRPLLV
YGAAQLAGAPLAKADVPAVAVELIHAYSLV
HDDLPAMDDDDLRRGQPTCHKAFDEATAIL
AGDTLHTRAFELLACHGDYRDGSRISLIQH
LCQAAGVDGMAAGQMQDMLAQGQQQTVAAL
EEMHYLKTGRLITASLQLGYFVAEKDDPSL
LANLTEFGDAIGLAFQIQDDILDVTAATEQ
LGKPSGSDEKLQKSTFPSLLGLEQSQQRAR
QLCDQAQQTLAGYGPRALPLQQLAQYIITR
NH
(Chlorella variabilis)
SEQ ID NO: 21
MGQVSAPVVEDMDICRQNLLNVVGERHPML
LAAANQIFSAGGKRLRPLIVLLVARATFPL
TGLSDITERHRRLAEISEMLHTASLVHDDV
LDECDVRRGKETVNSLYGTRVAVLAGDFLF
AQSSWFLANLDNMEVIKLISQVIADFADGE
ISQAASLFDAYIDLRRYLDKSFWKTASLIA
ASCRSAAVFSDCDTEARPPNRSCSLPPRLP
PPRRVALPAHLAGRCPWPPLLRRVQDEMVG
DGLLQLIQGRFKEEGSLQRALELVSLGGGI
DKARTLAREQGDLALASLACLPDTPAKRSL
ELMVDLVLERLY
(Ips confuses)
SEQ ID NO: 22
MFKLAQRLPKSVGSLGNQLSKVSNAPNQLM
SQMVPVTFQVMNTPIRHKSKSSAVPSSLSK
SMYEHNEEMKDAMKYMDEIYSEVMGQIEKV
PQYEEVKPILVRLRDAIDYTVPYGKRFKGV
HIVSHFKLLADPKFITPENVKLSGVLGWCA
EIIQAYFCMLDDIMDDSDTRRGKFTWYKLP
GIGLNAVTDVCLMEMFTFELLKRYFFQHPS
CADIHEIFRNLLFLTHMGQGCDFTFIDPVT
RKINFKEFTEENYTKLCRYKIIFSTFHNTL
ELTSAMANVYDPKKIQELDPVLMRIGMMHQ
SQNDFKDLYRDQGEVLKQVEKSVLGTDIRT
GQLTWFAQKALSICNDRQRKIIMDNYGKED
TKHSEAVREVYEELDLKGKFMEFEEESFQW
LKKEIPKINNGVPHKIFQDYTYGVFKRRPE
(Picea glauca)
SEQ ID NO: 23
MYTRCILKDKYSRFNLRRKFFTSTKSINAL
NGLPDSRNPRGESNGISQFKIQQVFPCKEY
IWIDRHKFHDVGFQAQHKRSITDEEQVDPF
SLVADELSILANRLRSMILTEIPKLGTAAE
YFFKLGVEGKRFRPMVLLLMASSLTIGIPE
VAADCLRKGLDEEQRLRQQRIAEITEMIHV
ASLLHDDVLDDADTRRGVGSLNFVMGNKLA
VLAGDFLLSRASVALASLKNTEVVELLSKV
LEHLVTGEIMQMTNTNEQRCSMEYYMQKTF
YKTASLMANSCKAIALIAGQPAEVCMLAYD
YGRNLGLAYQLVDDVLDFTGTTASLGKGSL
SDIRQGIVTAPILFALEEFPQLHDVINRKF
KKPGDIDLALEFLGKSDGIRKAKQLAAQHA
GFATFSVESFPPSESEYVKLCRKALIDLSE
KVITRTK
(Dendroctonus armandi)
SEQ ID NO: 24
MFSMKVCRNRSCREFLREARRTISKTSTDK
NSDAISRAQDHKLNVESDSNGSYSRWKKQM
HHNNIRALSTIQQSMVRPVQSSALVTKEQS
RDFMALFPDLVRELTEVGRSQELPDVMRRF
ARVLQYNTPTGKKNRGLIVLSTYRMLEDPE
KLTPENIRLASILGWCVEMVHAYFLILDDI
MDGSETRRGALCWYRQSGIGLSAINDAIMM
ENAVYLLLKRHLKDHPMYVPMMELFHEGTI
KTTLGQSLDAMCLDTNGKPKLDMFTMSRYT
SIVKYKTAYYSFQMPVAIAMYLAGMSDEEQ
HRQAKTILMEMGQFFQIQDDFLDCFGDPTV
TGKVGTDIQDGKCSWLAVVALQRASAAQRK
IMEEYYGRPEPESVAQIKNLYVDLCLPNTY
AIYEEESFNIIKTHIQQISKGLRHDLFFKI
MEKIYKREC
(Medicago sativa)
SEQ ID NO: 25
MATTTSHLTNVKSTVHFSCISNQHRSHLTT
KLKPTTVRMSMTQTPYWASLHADVEAHLKQ
TITIKEPLLVFEPMHHLIFTAPKTTVPALC
LAACELVGGQRQEAISAASALLLMEAATYT
HEHLPLSDRPGPKPGPMIDHVYGPNVELLT
GDGIVPFGFELLARSDGGENSERILKVMVE
ISRAVGSGGGVIDAQYMKTLGGGSDGDEIC
HVEEIRRVVEKYEGRLHSCGAVCGGVLGGG
CEEEIERLRKFGFYVGIIQGMIKWGFKEDH
KEVVEARNLAIQELKFFKDKEVDAIKTFLN
I
AAE
(Cannabis sativa AAE1)
SEQ ID NO: 26
MGKNYKSLDSVVASDFIALGITSEVAETLH
GRLAEIVCNYGAATPQTWINIANHILSPDL
PFSLHQMLFYGCYKDFGPAPPAWIPDPEKV
KSTNLGALLEKRGKEFLGVKYKDPISSFSH
FQEFSVRNPEVYWRTVLMDEMKISFSKDPE
CILRRDDINNPGGSEWLPGGYLNSAKNCLN
VNSNKKLNDTMIVWRDEGNDDLPLNKLTLD
QLRKRVWLVGYALEEMGLEKGCAIAIDMPM
HVDAVVIYLAIVLAGYVVVSIADSFSAPEI
STRLRLSKAKAIFTQDHIIRGKKRIPLYSR
VVEAKSPMAIVIPCSGSNIGAELRDGDISW
DYFLERAKEFKNCEFTAREQPVDAYTNILF
SSGTTGEPKAIPWTQATPLKAAADGWSHLD
IRKGDVIVWPTNLGWMMGPWLVYASLLNGA
SIALYNGSPLVSGFAKFVQDAKVTMLGVVP
SIVRSWKSTNCVSGYDWSTIRCFSSSGEAS
NVDEYLWLMGRANYKPVIEMCGGTEIGGAF
SAGSFLQAQSLSSFSSQCMGCTLYILDKNG
YPMPKNKPGIGELALGPVMFGASKTLLNGN
HHDVYFKGMPTLNGEVLRRHGDIFELTSNG
YYHAHGRADDTMNIGGIKISSIEIERVCNE
VDDRVFETTAIGVPPLGGGPEQLVIFFVLK
DSNDTTIDLNQLRLSFNLGLQKKLNPLFKV
TRVVPLSSLPRTATNKIMRRVLRQQFSHFE
(Cannabis sativa AAE3)
SEQ ID NO: 27
MEKSGYGRDGIYRSLRPPLHLPNNNNLSMV
SFLFRNSSSYPQKPALIDSETNQILSFSHF
KSTVIKVSHGFLNLGIKKNDVVLIYAPNSI
HFPVCFLGIIASGAIATTSNPLYTVSELSK
QVKDSNPKLIITVPQLLEKVKGFNLPTILI
GPDSEQESSSDKVMTFNDLVNLGGSSGSEF
PIVDDFKQSDTAALLYSSGTTGMSKGVVLT
HKNFIASSLMVTMEQDLVGEMDNVFLCFLP
MFHVFGLAIITYAQLQRGNIVISMARFDLE
KMLKDVEKYKVTHLWVVPPVILALSKNSMV
KKFNLSSIKYIGSGAAPLGKDLMEECSKVV
PYGIVAQGYGMTETCGIVSMEDIRGGKRNS
GSAGMLASGVEAQIVSVDTLKPLPPNQLGE
IWVKGPNMMQGYFNNPQATKLTIDKKGWVH
TGDLGYFDEDGHLYVVDRIKELIKYKGFQV
APAELEGLLVSHPEILDAVVIPFPDAEAGE
VPVAYVVRSPNSSLTENDVKKFIAGQVASF
KRLRKVTFINSVPKSASGKILRRELIQKVR
SNM
(Cannabis sativa AAE12)
SEQ ID NO: 28
MYMYQEVYLVPILSYLYLVVVLLPSIFFSF
RRMAFKSLDSVISSDIAALGIEPQLAHSLH
GRLAEIVSNHGSATPHTWRCISSHLLSPDL
PFSLHQMLYYGCYKDFGPDPPAWIPDAENA
ISTNVGKLLEKRGKEFLGVKYKDPISNFSD
FQEFSVTNPEVYWRTILDEMNISFSKPPEC
ILRENFSRDGQILNPGGEWLPGAFINPAKN
CLDLNCKSLDDTMILWRDEGKDDLPVNKMT
LKELRSEVWLVAYALKELELEGGSAIAIDM
PMNVHSVVIYLAIVLAGYVVVSIADSFAAP
EISTRLKISKAKAIFTQDLIVRGEKTIPLY
SRIVEAQSPLAIVIPSKGFSVSAQLRHGDV
SWHDFLNRANKFKNYEFAAVEQPIDAYTNI
LFSSGTTGEPKAIPWTQATPFKAAADAWCH
MDIQKGDVVAWPTNLGWMMGPWLVYASLLN
GASIALYNGSPLGSGFAKFVQDAKVTMLGV
IPSIVRTWKSTNCVAGYDWSTIRCFSSTGE
ASNIDEYLWLMGRAYYKPVIEYCGGTEIGG
GFVTGSLLQAQSLAAFSTPAMGCSLFILGS
DGYPIPKHKPGIGELALGPLMFGASKTLLN
ADHYDVYFKRMPSLNGKVLRRHGDMFELTS
KGYYHAHGRADDTMNLGGIKVSSVEIERIC
NEADEKVLETAAIGVPPLAGGPEQLVIAVV
LKNSDRTTVDLNQLRLSFNSAVQKKLNPLF
RVSRVVPLSSLPRTATNKVMRRILRQQFTQ
LDKSSKI
(Ziziphus jujube)
SEQ ID NO: 29
MAHKSLDGITASDIEALGIEPEVAKSLHGR
LTKIIRNYGTATPDTWSNISRHILSPDLPF
SFHQMMYYGCYKDFGPDPPAWIPDLEAAVS
TNVGQLLERQGKEFLGSRYKDPISSFSDFQ
EFSVKNPEVYWKTILDEMNVSFSIPPQCIL
RENVSGERHFSHPGGEWLPGAFVNPANNCL
SLNYKRNLDDSMVLWRDEGKDDLPINKMTL
KELREEVWLVAHALEKLGLDKGSAIAIDMP
MDVRSVIIYLAIVLAGYVVVSIADSFAPLE
ISTRLRISQAKAIFTQDLIIRGEKCIPLYS
RIVEAESPMAIVIPTRGSSFSIKLRDGDVA
WNDFLERVGDFKKIEFAAVDQPIEAFTNIL
FSSGTTGEPKAIPWTHATPFKAAADAWCHM
DIQKGDVVCWPTNLGWMMGPWLVYASLLNG
ASIALYNGSPLGSGFAKFVQDAKVTMLGVI
PSIVRTWKSSNCVAGYDWSTIRCFGSTGEA
SNVDEYLWLMGRACYKPVIEYCGGTEIGGG
FVSGSLLQAQSLAAFSTPAMGCSLYILGSN
GLPIPQNQPGIGELALDPLMFGASRTLLNA
DHYDVYFKGMPVWNGKVLRRHGDMFELTSR
GYYHAHGRADDTMNIGGIKVSSVEIERICN
EVDDSVLETAAIGVPPLGGGPEQLVIAVVF
KDSNNPKEDLNQLRISFNSAVQKKLNPLFR
VSRVVPLLSLPRTATNKVMRRILREQFSQH
DQSSKI
(Trema orientale)
SEQ ID NO: 30
MGYKSLDSVTASDIAALGIDPELAETLHGR
LADVIRNYASATPPDTWRYVSANILSPHLP
FSFHQMMYYGCYQDFGPDPPAWIPDLENAI
STNVGKLLERRGKEFLGSSYKDPISNFSDF
QEFSVTNPEVYWKTILDEMNVSFSKPPQCI
LLENFPGDGKLLHPGGEWLPGAYVNPAKNC
LSLNSKRSLDDTMIIWRDEGKDDLPVNKMT
LEELRSEVWLVAYALKELGLEGGSAIAIDM
PMNVHSVVIYLAIVLAGYVVVSIADSFAAR
EISTRLKISNAKAIFTQDLIIRGEKSIPLY
SRIVEAQSPTAIVIPTRGSSFSAKLRQDDI
SWHDFLERAKAFKKREFAAIEQPVDAYTNI
LFSSGTTGEPKAIPWTHATPFKAAADAWCH
MDIQKGDVVAWPTNLGWMMGPWLVYASLLN
GASIALYNGSPLGSGFAKFVQDAKVTMLGV
IPSIVRTWKSTNSIASYDWSTIRCFSSTGE
ASNVDEYLWLMGRACYKPVIEYCGGTEIGG
GFVTGSLLQAQSLAAFSTPAMGCSLFVLGS
DGYPIPKNKPGIGELALGPLMLGASKTLLN
ADHYDVYFKGMPSWNGKVLRRHGDMFEFTS
RGYYRAHGRADDTMNLGGIKVSSVEIERIC
NEADDEVLETAAIGVPPPTGGPEKLVIAVV
FKNPENTGADLNQLRLSFNSAVQKKLNPLF
RVSHVVPLPSLPRTATNKVMRRILRQQLAQ
LDQSSKI
(Parasponia andersonii)
SEQ ID NO: 31
MGYKSLDSVTASDIAALGIDPELAETLHGR
LADVIRNYASATPPDTWRYVSANILSPHLP
FSFHQMMYYGCYQDFGPDPPAWIPDLENAI
STNVGKLLERRGKEFLGSSYKDPISNFSDF
QEFSVTNPEVYWKTILDEMNISFSKPPQCI
LRENFPGDGQLLHPGGEWLPGAYVNPAKNC
LSLNSKRSLDDTMIIWRDEGKDDLPVNKMT
LEEFRSEVWLVAYALKELGLERGSAIAIDM
PMNVHSVVIYLAIVLAGYVVVSIADSFAAR
EISTRLKISKAKAIFTQDLIIRGEKSIPLY
SRIVEAQSPTAIVIPTRGFSFSAKLRQGDI
SWHDFLERAKAFEKREFAASEQPVDAYTNI
LFSSGTTGEPKAIPWTQATPFKAAADAWCH
MDIQKGDVVAWPTNLGWMMGPWLVYASLLN
GASIALYNGSPLGSGFAKFVQDAKVTMLGV
IPSIVRTWKSTNSVAFYDWSTIRCFSSTGE
ASNVDEYLWLMGRACYKPVIEYCGGTEIGG
GFVTGSLLQAQSLAAFSTPAMGCSLFILGS
DGYPIPKNKPGIGELALGPLMLGASKTLLN
FDHYDVYFKGMPWWNGKVLRRHGDMFEFTS
SGYYRAHGRADDTMNLGGIKVSSVEIERIC
NEADDEVLETAAIGVPPPTGGPEKLVIAVV
FKNPENTGADLNPLRLSFNSAVQRKLNPLF
RVSHVVPLPSLPRTATNKVMRRILRQQLAQ
LDQSSKI
(Prunus avium)
SEQ ID NO: 32
MAYKSLDHVTVSDIEALGIESEAAKRLHAS
LTNIIQNYGPATPDTWRNITAHVLSPELPF
SFHQMLYYGCYKDFGPDPPAWLPDSETTNL
TNVGQLLERRGKEFLGSRYKDPMSSFSDFQ
EFSVSNPEVYWKAVLDEMNASFSIPPQCIL
RENLSGDGQLSVLGGQWLPGAFGNPAKNCL
SLNRKRSLNDTMVIWRDEGNDDLPLNKMTL
KELRTEVWLVAHALKALGLEKGSAIAIDMP
MHVNSVIIYLAIVLAGYVVVSIADSFAPPE
ISTRLKISEAKAIFTQDLIVRGEKSLPLYS
KIVAAQSPMAIVILTKGSNSSMKLRDGDIS
WHDFLETVKDFKEDEFAAVEQPIEAFTNIL
FSSGTTGEPKAIPWTHATPFKAAADAWCHM
DIQIGDVVSWPTNLGWMMGPWLVYASLLNG
ASIALYNGSPLGSGFPKFVQDAKVTMLGVI
PSIVRTWKSTNSVSGYDWSTIRCFGSTGEA
SNVDEYLWLMGRARYKPIIEYCGGTEIGGG
FVSGSLLQAQSLAAFSTPAMGCSLFILGND
GVPIPQNEPGVGELALGPLIFGASSTLLNA
DHYDVYFKGMPFWNGKVLRRHGDVFERTSR
GYYHAHGRADDTMNLGGIKVSSVEIERICN
EVDSEVLETAAIGVPPAVGGPEQLVLAVVF
KNSDNQTADLNQLRTSFNSAVQKKLNPLFK
VSRVVPLPSLPRTATNKVMRRILREQFAQL
DQSAKL
(Morus notabilis)
SEQ ID NO: 33
MTDKSLDGVTASNIAALGIAPDVADGLHGR
IAEVVRIYGPANPDTWRQISTRVLSPDLPF
AFHQMLYHSCFNGFGPDPPAWIPDPEAAIL
TNVGKLLERRGKEFLGSRYKDPISNFSDFQ
EFSVTNPEVYWRTIFNEMNVSFSNPPECIF
HENVPGGGQVSHPGGQWLPGAYVNPAMNCL
SVNSKRSLDDASIVWRDEGKDDLPVNTMTL
EELRSEVWLVAHALKELGLERGSAIAIDMP
MHVHSVVIYLAIVLAGYVVVSIADSFAAGE
ISTRLKISKAKAIFTQDLIIRGEKSIPLYR
RVVEAQSPMAIVIPTRGSSFSTQLRHGDIG
WHDFLERVKEFKKCEFTAAEQPVDAFTNIL
FSSGTTGDPKAIPWTQATPFKAAADAWCHM
DIQKGDVVAWPTNLGWMMGPWLVYASLLNG
ASIALYNGSPLGSSFAKFIQDAKVTMLGVI
PSIVRTWKSMNSVSGYDWSTIRCFGSTGEA
SNVDEYLWLMGRACYKPVIEYCGGTEIGGG
FVTGSLLQAQALAAFSTPAMGCSLFILGSD
GYPIPKNKPGIGELALGPVMFGSSMTLLNA
DHYDVYFKGMPLWNGKVLRRHGDMFEITSR
GYYRAHGRADDTMNLGGIKVSSVEIERLCN
EVDNSILETAAIGVPPPAGGPEQLVIAVVF
KDPDSNITTDLNQLRMSLNSAVQKKLNPLF
RVSRVVPLQSLPRTATNKVMRRILRQQFVQ
LDQTSKM
(Rosa chinensis)
SEQ ID NO: 34
MSYKSLDAVTVADIAALGIEPELANRLHGS
LAKIIADHGAATPDTWRSITGHVLSPDLPF
SFHQMMYYGCYKDFGPDPPAWLPDPETAVL
TNAGQLLERRGKEFLGSQYKDPISSFSDFQ
EFSVSNPEVYWKTVLDEMNVSFYKPPQCIL
RENLSGDGHLLVPGVQWLPGACVNPAKNCL
SLNSKRSLNDTMVVWRDEGKDDLPLNKMTL
KELRAEVWLVAHALQAQGLEKGSAIAIDMP
MNVISVVIYLAIVLAGYVVVSIADSFAPPE
ISTRLKISEAKAIFTQDVIVRGEKSLPLYS
KIVDAQSPMAIVLLTRGSKSSVKLRDGDIS
WHDFLNTVKDFKDEFAAVEQPVEAFTNILF
SSGTTGDPKAIPWTHSTPFKAAADANCHMD
IRKGDVIAWPTNLGWMMGPWLVYASLLNVA
SIALYNGSPLGPGFSKFVQDAKVTMLGVIP
SIVRTWKSTNSTSGYDWSAIRCFSSTGEAS
NVDEYLWLMGRAGYKPIIEYCGGTEIGGAF
VSGSLLQAQSLASFSTPAMGCSLFILGTDG
SPIPQNEPGVGELALGPLMFGASSTLLNAD
HYEVYFKGMPLWNGKVLRRHGDLFERTSRG
YYHAHGRADDTMNLGGIKVSSVEIERICNA
IDTNILETAAIGVPPAGGGPEQLVIAVVFK
NSDNPPADLNQLRASFNSAVQKKLNPLFKV
SRVVPLPSLPRTATNKVMRRILRQQFAQVD
QGAKL
(Citrus sinensis)
SEQ ID NO: 35
MATYNYKALDCITSCDIEALGIPSKLAEQL
HEKLAEIVNTHGAATPATWQNITTHILSPD
LPFSFHQLLYYGCYKDFGPDPPAWIPDPEA
AKVTNVGKLLQTRGEEFLGSGYKDPISSFS
NFQEFSVSNPEVYWKTVLNEMSTSFSVPPQ
CILRENPNGENHLSNPGGQWLPGAFVNPAK
NCLSVNSKRSLDDIVIRWRDEGDSGLPVKS
MTLKELRAEVWLVAYALNALGLDKGSAIAI
DMPMNVNSVVIYLAIVLAGYIVVSIADSFA
SLEISTRLRISKAKAIFTQDLIIRGDKSIP
LYSRVIDAQAPLAIVIPAKGSSFSMKLRDG
DISWFDFLERVRKLKENEFAAVEQPVEAFT
NILFSSGTTGEPKAIPWTNATPFKAAADAW
CHMDIRKADIVAWPTNLGWMMGPWLVYASL
LNGASIALYNGSPLGSGFAKFVQDAKVTML
GVVPSIVRTWKSTNCIDGYDWSSIRCFGST
GEASNVDEYLWLMGRALYKPVIEYCGGTEI
GGGFITGSLLQAQSLAAFSTPAMGCKLFIL
GNDGCPIPQNVPGMGELALSPLIFGASSTL
LNANHYDVYFSGMPSRNGQILRRHGDVFER
TSGGYYRAHGRADDTMNLGGIKVSSVEIER
ICNAVDSNVLETAAIGVPPPDGGPEQLTIV
VVFKDSNYTPPDLNQLRMSFNSAVQKKLNP
LFKVSHVVPLPSLPRTATNKVMRRVLRKQL
AQLDQNSKL
(Citrus clementina)
SEQ ID NO: 36
MATCNYKALDCITSYDIEALGIPSKLAEQL
HEKLAEIVNTHGAATPATWQNITTHILSPD
LPFSFHQLLYYGCYKDFGPDPPAWIPDPEA
AKVTNVGKLLETRGEEFLGSGYKDPISSFS
NFQEFSVSNPEVYWKTVLNEMSTSFSVPPQ
CILRENPNGENHLSNPGGQWLPGAFVNPAK
NCLSVNSKRSLDDIVIRWCDEGDGGLPVKS
MTLKELRAEVWLVAYALNALGLDKGSAIAI
DMPMNVNSVVIYLAIVLAGYIVVSIADSFA
SLEISARLRISKAKAIFTQDLIIRGDKSIP
LYSRVIDAQAPLAIVIPAKGSSFSMKLRDG
DISWLDFLERVRKLKENEFAAVEQPVEAFT
NILFSSGTTGEPKAIPWTNATPFKAAADAW
CHMDIRKADIVAWPTNLGWMMGPWLVYASL
LNGASVALYNGSPLGSGFAKFVQDAKVTML
GVVPSIVRTWKSTNCIDGYDWSSIRCFGST
GEASNVDEYLWLMGRALYKPVIEYCGGTEI
GGGFITGSLLQAQSLAAFSTPAMGCKLFIL
GNDGCPIPQNVPGMGELALSPLIFGASSTL
LNANHYDVYFSGMPSWNGQILRRHGDVFER
TSGGYYRAHGRADDTMNLGGIKVSSVEIER
ICNAVDSNVLETAAIGVPPPDGGPEHLTIV
VVFKDSNYRPPDLNQLRMSFNSAVQKKLNP
LFKVSHVVPLPSLPRTATNKVMRRVLRKQL
AQLDQNSKL
(Arachis duranensis)
SEQ ID NO: 37
MAYKSLTSITVSDIESVGISTEVASAFHRR
LKEIIATHGAGTPATWHNITNTILTPDLPF
SFHQMLYYACYIDFGPDPPAWIPDPECALS
TNVGQLLERRGKEFLGSAYKDPISSFSDFQ
KFSVSNPEVFWKNVLDEMNISFSTPPECIL
RENLPGESSLTHPGGQWLPGASINPAKNCL
VENAKRSLNDTAIIWRDEHHDDLPVQRMTF
KELQEEVWLVAYALEALGLEKGSAIAIDMP
MHVKSVVIYLAIVLAGYVVVSIADSFAAGE
ISTRLNISNAKVIFTQDLIIRGDKSIPLYS
RVVEAKSPLAVVIPTRGSEFSMELRNGDFS
WHDFLDRANSLKGKEFVAVEQPVEAFTNIL
FSSGTTGEPKAIPWTNITPLKAAADAWCHL
DIRKGDVVSWPTNLGWMMGPWLVYASLING
ASMALYNGSPLGSGFAKFVQDAKVTMLGVI
PSIVRSWKSANSTSGYDWSAIRCFGSTGEA
SNVDEYLWLMGRALYKPVIEYCGGTEIGGG
FITGSLLQPQSVAAFSTPAMCCSLFILDEE
GHPIPQDVPGMGELALGPIMFGASITLLNA
DHYAVYFKGMPVYNGKVLRRHGDVFERTAK
GYYHAHGRADDTMNLGGIKVSSVEIERLCN
GVDSSILETAAIGVPPSGGGPEQLVVAVVF
KNPSTTTQDLHQLRISFNSALQKKLNPLFR
VSRVVSLPSLPRTASNKVMRRVLRQQLSEN
NQSSKI
(Quercus suber)
SEQ ID NO: 38
MGYKALDRITRSDIEEEVGIAAAAGVAERI
HERLTEIVRNYGADTPDTWRSICERVLSPD
LPFSLHQMMFYGCYNGYGTDPPAWIPDPKT
AILTNVGQLLERRGKEFLGSKYKDPISSFS
DLQEFSVSNPEVYWKTVLDEMSISFSVPPQ
CILRDSPFGESHSSYPGGQWLPGAFLNPAE
NCLSLNSKRSLEDIAVIWRDEGDDILPVNR
MTVREFRAEVWLVAHAIKTLGLDKGSAIAI
DMPMNVNSVVIYLAIVLAGYVVVSIADSFA
PREISTRLKISEAKAIFTQDLIIRGDKSIP
LYSRIVEAQSPMAVVIPARGSSFSMKLRDG
DISWHDFLGRVKNFKECEFAAVEQPVEAFT
NILFSSGTTGEPKAIPWTSATPLKAAADAW
CHLDIQKGDVVAWPTNLGWMMGPWLVYASL
LNGASMALYNGSPLSSGFAKFVQDAKVTML
GVIPSIVRAWKSTNCMAGYDWSAIRCFGST
GEASNVDEYLWLMGRACYKPIIEYCGGTEI
GGGFITGSFLQAQSLAAFSTPAMGCSLFIL
GSDGYPIPENVPGIGELALGPLMFGASNKL
LNADHHDVYFKGMPLWKGRVLRRHGDVFER
TSRGYYHAHGRADDTMNLGGIKVSSVEIER
ICNAADNSVLETAAIGVPPSGGGPEQLVIA
VVFKESENMTADLNQLRISFNSAVQKKLNP
LFRVSQVVPLSSLPRTASNKVMRRVLRQQL
TQGDRNPKL
(Theobroma cacao)
SEQ ID NO: 39
MVYKSLDSVTVKDIEASGISSQLAEEIHRK
VTEIVDGYGAATPESWNRISKHVLTPNLPF
SLHQMMYYGCYKDFGPDPPAWMPDPESALL
TYVGLLLEKHGKEFLGSKYKDPISSFSHLQ
EFSVSNPEVYWKTVLDEMCVNFSVPPDCIL
HESTSEESRILNPGGKWLPGAFVNPAKNCL
IVNSKRGLDDIVIRWRDEGDDDLPVKSMTL
KELQLEVWLVAHALNALGLERGSAIAIDMP
MNVYSVIIYLAIVLAGYIVVSIADSFAPLE
ISTRLKISEAKAIFTQDLIIRGEKSIPLYS
RVVEAEAPMAIVIPARGFSCSAKLRDGDIS
WSDFLERVRELKGDVFEAVEQPVEAFTNVL
FSSGTTGEPKAIPWTHVTPLKAAADAWCHM
DIHSGDIVAWPTNLGWMMGPWLVYASLLNG
ASMALYNGSPLSSGLAKFVQDAKVTMLGVI
PSIVRAWKSTNCVAGYDWSSIRCFSSTGEA
SNVDEYLWLMGRACYKPIIEYCGGTEIGGG
FVSGSFLQPQSLAAFSTPAMGCRLFILGDD
GHPIPQDAPGMGELALGPLMFGSSSTLLNA
SHYDVYFKEMPSWNGLILRRHGDVFERTSR
GYYHAHGRADDTMNIGGIKVSSVEIERICN
AVDSSVLETAAIGVPPADGGPERLVIAVVF
KDPDNATPDLNQLRKSFNSAVQKNLNPLFR
VSHVVALSALPRTASNKVMRRVLRKQLAQV
DQNSKL
(Jatropha curcas)
SEQ ID NO: 40
MAHNALGAISVSDIEALGISSELAEKLYTH
VSQIINNYGSATPETWSRISKHVLTPDLPF
SFHQMMFYGCYKDFGPDPPAWLPDPKSAAL
TNVGQLLQRRGKEFLGEGYVDPISSFSAFQ
EFSVSNPEVYWKTVLDEMDVAFSVPPQCIL
REDLSGESSFLNPGGQWLPGAYVNPAKNCL
SLNSKRILDDTVIRWRCEGSDDLPVSSMTL
EELRTEVWLVAYALNSLGLDRGSAIAIDMP
MNVKAVVIYLAIVLAGYVVVSIADSFAPLE
ISTRLKISKAKAIFTQDLIIRGDKNIPLYS
RVVDAQSPMAIVIPTKGSSFSMKLRDGDIS
WHDFLEKVQNLRGNEFAAVEQPIEAFTNIL
FSSGTTGEPKAIPWTSATPFKAAADAWCHM
DIRKGDIVAWPTNLGWMMGPWLVYASLLNG
ACIALYNGSPLGSSFAKFVQDAKVTMLGVI
PSIVRTWKTANTTAGYDWSAIRCFGSTGEA
SNVDEHLWLMGRALYKPIIEYCGGTEIGGG
FVSGSFLQPQSLAAFSTPAMGCSLFILGDD
GHPIPHDVPGIGELALGPLMFGASSSLLNA
DHYNVYYKGMPVWNGKILRRHGDVFERTSR
GYYHAHGRADDTMNLGGIKVSSVEIERICN
VVDSSILETAAIGVPPPQGGPEQLVIAVVF
KNLENSTTDLEQLRKSFNSAVQKKLNPLFR
VSRVVPHPSLPRTASNKVMRRILRQQFVQQ
EQNSKL
(Populus trichocarpa)
SEQ ID NO: 41
MASLHYKALDSISVSDIEALGISSSIALQL
YEDISEIINTHGPSSPQTWTLLSKRLLHPL
LPFSFHQMMYYGCFKDFGPDPPAWSPDPEA
AMLTNVGQLLERRGKEFLGSAYKDPISSFS
NFQEFSVSNPEVYWKTILDEMSISFSVPPQ
CILSENTSRESSLANPGGQWLPGAYVNPAK
TCLTLNCKRNLDDVVIRWRDEGNDDMPVSS
LTLEELRSEVWLVAYALNALGLDRGSAIAI
DMPMNVESVIIYLAIVLAGHVVVSIADSFA
PLEISTRLKISEAKAIFTQDLIIRGDKSIP
LYSRVVHAQAPMAIVLPTKGCSFSMNLRDG
DISWHDFLEKATDLRGDEFAAVEQPVEAFT
NILFSSGTTGEPKAIPWTHLTPFKAAADAN
CHMDIRKGDIVAWPTNLGWMMGPWLVYASL
LNGASIALYNGSPLGSGFAKFVQDASVTML
GVIPSIVRIWKSANSTSGYDWSAIRCFAST
GEASSVDEYLWLMGRAQYKPIIEYCGGTEI
GGGFVSGSLLQPQSLAAFSTPAMGCSLFIL
GDDGHPIPQNVPGMGELALGPLMFGASSTL
LNADHYNVYFKGMPLWNGKILRRHGDVFER
TSRGYYHAHGRADDTMNLGGIKVSSVEIER
VCNAVDSNVLETAAVGVPPPQGGPEQLVIA
VVFKDSDESTVDLDKLRISYNSAVQKKLNP
LFRISHVVPFSSLPRTATNKVMRRVLRQQL
SQQDQNSKL
(Hevea brasiliensis)
SEQ ID NO: 42
MSSYKALDAISVSDIEALGISSKLADKLYK
DVADIIANYGASTPQTWTHISKHVLNPDLP
FSLHRMMFYACYKDFGSDPAAWSPDPKTAA
LTNVGQLLERRGKEFLGSLYVDPISSFSAF
QEFSVSNPEVYWKTVLDEMSISFSVPPQCI
LLENPESPGGQWLPGAYVNPARNCLSLNRE
RTLDDTVITWRDEGSDDLPLSSMTLGELRT
EVWLVAYALNTLGLDRGSAIAIDMPMNVKS
VVIYLAIVLAGYAVVSIADSFASPEMSTRL
KISEAKAIFTQDLIIRGDKSIPLYSRVVDA
QSPMAIVIPTKGSSFSMKLRGGDISWHDFL
ERVENIRGDEFAAVEQPIEAFTNILFSSGT
TGDPKAIPWTNATPFKAAADAWCHMDIRRG
DVVAWPTNLGWMMGPWLVYASLLNGACIAL
YNGSPLGSGFAKFVQDAKVTMLGVIPSIVR
TWKSANSTAGYDWSAIRCFGSTGEASNVDE
YLWLMGRAHYKPIIEYCGGTEIGGGFVSGS
LLQPQSLAAFSTPAMGCSLFILGDDGHPFP
QNVPVMGELALGPLMFGASSSLLNANHYNV
YYKGMPVWNGKILRRHGDVFEHTSRGYYRA
HGRADDTMNLGGIKVSSVEIERICNAVDSS
ILETAAIGVPPPQGGPERLVIAVVFNDPDN
STTDLEQLRKSFNSAVQKKLNPLFRVSHVV
ALPSLPRTATNKVMRRILRQQFVQQEQNSK
L
(Vitis vinifera)
SEQ ID NO: 43
MAGKTLDSITSQDIAALGIPSEEAEKLHQT
LLQIITSCGAATPQTWSRISKELLNPDLPY
SLHQMMYYGCYSHFGPDPPAWLPDPENVML
INVGQLLERRGKEFLGSRYKDPISSFSDFQ
KFSVSNPEVYWKTVLDELSISFSVPPQCVL
YDNPSRENGLSYPGGQWLPGAFINPARNCL
SVNDKRTLDDTVVIWHDEGDDGMPINRMTL
EELRREVWSVAYALDTLGLEKGSAIAIDMP
MNASSVVIYLAIVLAGYIVVSIADSFASRE
ISTRLKISNAKAIFTQDFIIRGDKSLPLYS
RVVDAQSPTAIVIPAGGSSFSMKLRDGDMS
WHDFLQRAINSRDDEFAAIEQPIEAFMNIL
FSSGTTGEPKAIPWTNATPLKAAADAWCHM
DIRKGDIVAWPTNLGWMMGPWLVYASLLNG
ATIALYNGAPLGSGFAKFVQDAKVTMLGVI
PSIVRTWKSTNCTAGLDWSSIRCFASTGEA
SSVDEYLWLMGRAQYKPIIEYCGGTEIGGG
FVTGSLLQAQSLASFSTPAMGCSLFIIGDD
GNLLPQDASGMGELALGPLMFGASTTLLNA
DHYDVYFKGMPIWNGKVLRRHGDVFERTSR
GYYRAHGRADDTMNIGGIKVSSVEIERICN
TVHSSVLETAAIGMPPPAGGPERLMIVVVF
KDSNNSIPDLNELRIAFNSEVQKKLNPLFR
VSHTVPVPSLPRTATNKVMRRVLRQQLAQL
SSTSKF
(Manihot esculenta)
SEQ ID NO: 44
MDNKVLDAISVSDIEALGISSPLAHKLCKD
VADIVANYGAATPQTWTHISKHVLHPDLPF
SFHQMMFNACYKDFGTDPPAWSPDLKSAAL
TNVGHLLERRGKEFLGSLYVDPISSFSAFQ
EFSVSNPELYWKTVLDEMNISFSVPAQCIL
LENSYGESPGGQWLPGAYVNPAKNCLSLNC
KRTLDDTVIRWRDEGSDELPLSSMTLDELR
TEVWLVAYALNRLGLDRGSAIAIDMPMNVK
SVVIYLAIVLAGYVVVSIADSFAPLEIATR
LKISEAKAIFTQDLIIRGDKSIPLYSRVVD
AQSPMAVVIPAKGSSFSMKLRDGDISWHDF
LERVENRRGDEFAAVEQPIEAFTNILFSSG
TTGEPKAIPWINATPFKAAADANCHMDIHK
GDVVAWPTNLGWMMGPWLVYASLLNGACIA
LYNGSPLGSGFAKFVQDAEVTMLGVIPSIV
RTWKSANSTAGYDWSSIRCFGSTGEASNID
EYLWLMGRAHYKPVIEYCGGTEIGGGFVSG
SLLQPQSLAAFSTPAMGCSLFILGDDGHPI
PHNAPGMGELALGPLMFGASSSLLNADHYN
VYFKGMPVWNGKILRRHGDVFERTSRGYYH
AHGRADDTMNLGGIKVSSVEIERICNAVDN
SILETAAIGVPPSQGGPERLVIAVVFKNPD
NTTRDLEQLRKTFNSAVQKKLNPLFRVSHV
VALPTLPRTATNKVMRRILRQQFVQQEQTA
KL
(Nicotiana attenuate)
SEQ ID NO: 45
MAHQNYKGLDSVTVADVEALGIASELAGEI
HEKLTRIVRNYSATTPQTWHHISKEILTPK
LPFSLHQMMYYGCYKDFGPDPPAWLPDSKN
VGLTNIGQLLERRGKEFLGSNYEDPISSFS
DFQRFSVSEPEVYWKTILEEMNVSFSVPPE
CILRESPSHPGGQWLPGARVNPAKNCLSFR
KRTLSDVAIVWRSEGNDEAPVEKMTLKELC
ESVWAVAYALETLGLEKGSAIAIDMPMDVN
SVVIYLAIVLAGYVVVSIADSFAPSEISTR
LILSKAKAIFTQDFIFRGDKKIPLYSRVVD
ARSPTAIVIPNRASSLSIQLRDGDISWPEF
LERVKDSRGLEFVAVEQPITAFTNILFSSG
TTGEPKAIPWSLLSPFKSAADGWCHMDIKK
GDVVAWPTNLGWMMGPWLVYASLLNGASIA
LYNGSPLDSGFAKFVQDAKVTMLGVIPSIV
RTWKAKNSPDGFDWSTIRCFGSTGEASSVD
EYLWLMGRAEYKPIIEYCGGTEIGGSFVSG
SLLQPQSLAAFSTAVMGCSLHILGEDGLPI
PSDVPGTGELALGPLMFGASSTLLNADHNE
IYFKGMPVLNGKVLRRHGDVFERTSKGYYH
AHGRADDTMNLGGIKVSSLEIERICNAADE
NILETAAVGVPPAGGGPEKLVIAVVFKDSA
NLEHNMDKLMISFNTALQRKLNPLFKVSSI
VPLPLLPRTATNKVMRRVLRQQFSQAEQGS
KL
(Solanum pennellii)
SEQ ID NO: 46
MANQNYRTLDSVTVADVEALGIPTELAEKL
HEELTRIVRNYGSVTPQTWHHISKELLTPN
LPFSFHQMMYYGCYKDFGSDPPAWLPDPKT
ARLTNIGQLLERRGMEFLGSKYDDPISSFS
DFQRFSVSDQEVFWKTILEEMNISFSVPPE
CILRESPSHPGGQWLPGSRANPAKNCLSLR
KRTLSDVAIIWRSEGNDEAPVEKMTCQELR
ESVWEVAYALESLGLEKGSAIAIDMPMDVN
SVVIYLAIVLAGYVVVSIADSFAPSEISTR
LILSKAKAIFTQDFIPRGEKKIPLYSRVVE
AHSPMAIVIPNRVSSLSIELRDGDISWPDF
LDRVKDSKGLEFVAVEQPIDAFTNILFSSG
TTGDPKAIPWTLLTPFKAAADGWCHMDIKN
GDVVAWPTNLGWMMGPWLVYAALLNGASIA
LYNGSPLGSGFAKFVQDAKVTMLGVIPSIV
RTWKAKNSPDGYDWSTIRCFGSTGEASSVD
EYLWLMGRAEYKPIMEYCGGTEIGGSFVSG
SMLQPQSLAAFSTAVMGCSLHILGDDGFPI
PSDVPGIGELALGPLMFGASSTLLNADHNE
IYFKGMPVLNGKVLRRHGDVFERTSKGYYH
AHGRADDTMNLGGIKVSSLEIERICNVVDE
NILETAAVGVPPAAGGPEKLVIAVVFKDSD
NLEQKLVNLLISFNTALQRKLNPLFKVSSI
VPLPSLPRTATNKVMRRVLRQQFSQADQGS
RL
(Nelumbo nucifera)
SEQ ID NO: 47
MAIKSLDCVTVEDITGLGISSDAAKKLHGD
LTEILRENANSAADTWKKISKRILNPNLPF
AFHQMMYYGCFKDFGSDPPAWIPDQETAIL
TNVGRFLEKRGKEFLGSKYKDPITSFLDFQ
EFSVSNPEVYWKMVLDEMNISFSVPPSCIL
YEHTSEGGHLSYPGGQWLPGAILNCAENCL
NLNGKRSLNDTMIIWRDEGDDNLPVKHMML
KQLRSEVWLVAYALDTLGLAKGSAIAIDMP
MNVTAVVIYLAIVLAGYIVVSIADSFAPLE
ISTRLKISNAKAIFTQDVIIRGDKILPLYS
RVVDAQAPLAIVVPSRGSSLKMELRGCDMS
WHAFLERVEHFKKDEFAAVQQPVDAFTNIL
FSSGTTGEPKAIPWTHATPLKAAADAWCHM
DIQKGDVVAWPTNLGWMMGPWLVYASLLNG
ASMALYNGSPLGSGFAKFVQDAKVTMLGVV
PSIVRAWKNTNCTAGFDWSSIRCFSSTGEA
SNVDEYLWLMGRAHYKPVIEYCGGTEIGGG
FVSGSLLQAQSLAAFSTPAMGCTLFILCSD
GNPILQNTPGIGELALAPIMLGASNTLLNA
NHYDVYFRGMPMWNGKVLRRHGDEFECTSK
GYYRAHGRADDTMNLGGIKVSSIEIERICN
GVDDTILETAAIGVPPVGGGPEKLAIAVVF
KDSNSLPDVDQLKMKFNSSLQKKLNPLFRV
SAVVPVSSLPRTASNKVMRRVLRQQFSQLY
QASTSRIASGFLLQSPPQRPSTSL
(Momordica charantia)
SEQ ID NO: 48
MDYKTLDSITVIDIEALGVASEVAEKLHGL
LSEIIRSHGNGTPETWRHISKRVLSPDLPF
SFHQMMYYGCYKHYGPDPPAWIPEPENAVF
TNVGQLLKRRGKEFLGSNYRDPLSSFSSFQ
EFSVSNPEVYWRTMLDEMHITFSKPPHCIL
QMNDSTESQFSSPGGQWLPGAVFNPAKDCL
SLNENRSLDDVAIIWRDEGCDNLPVKRLTL
GELRTDVWLIAHALNSIGFEKGTAIAIDMP
MNVNAVVIYLGIVLAGHVVVSIADSFSARE
ISTRLDISKAKAIFTQDLIIRGDKSIPLYS
RVVDAQSPMAIVIPSRSTGFSRKLRDEDIS
WHAFLERVEDLRGVEFAAVEQAAESFTNIL
FSSGTTGEPKAIPWTLVTPLKAAADAWCYM
DIHKGDVVAWPTNLGWMMGPWLVYASLLNS
ASMALYNGSPLGSGFVKFVQDAKVTMLGVI
PSIVRSWKSTNCTSGYDWSSIRCFASTGEA
SNVDENLWLMGRACYKPVIEICGGTEIGGG
FITGSLLQPQALAAFSTPAMGCSLFILGND
GFPIPQNMPGIGELALGPFLFGASSTLLNA
DHYDIYFKGMPHWNGMVLRRHGDVFERSPR
GYYRAHGRADDAMNLGGIKVSSVEIERICN
TIDDSILETAAIGVPPLGGGPEQLVIAVVL
KNPGETSPDLDKLKLCFNSSLQKNLNPLFR
VHRVVPYPSLPRTATNKVMRRILRQQLAVE
RRTKL
OLS
(Cannabis sativa)
SEQ ID NO: 49
MNHLRAEGPASVLAIGTANPENILLQDEFP
DYYFRVTKSEHMTQLKEKFRKICDKSMIRK
RNCFLNEEHLKQNPRLVEHEMQTLDARQDM
LVVEVPKLGKDACAKAIKEWGQPKSKITHL
IFTSASTTDMPGADYHCAKLLGLSPSVKRV
MMYQLGCYGGGTVLRIAKDIAENNKGARVL
AVCCDIMACLFRGPSESDLELLVGQAIFGD
GAAAVIVGAEPDESVGERPIFELVSTGQTI
LPNSEGTIGGHIREAGLIFDLHKDVPMLIS
NNIEKCLIEAFTPIGISDWNSIFWITHPGG
KAILDKVEEKLHLKSDKFVDSRHVLSEHGN
MSSSTVLFVMDELRKRSLEEGKSTTGDGFE
WGVLFGFGPGLTVERVVVRSVPIKY
(Humulus lupulus)
SEQ ID NO: 50
MSSSITVDQIRKAQRAEGPATILAIGTATP
ANFIIQADYPDYYFRVTKSEHMTNLKKRFQ
RICDRTMIKKRHLVLSEDHLKENPNMCEFM
APSLDVRQDILVVEVPKLGKEACMKAIKEW
DQPKSKITHFIFATTSGVDMPGADYQCAKL
LGLSSSVKRVMMYQQGCFAGGTVLRIAKDI
AENNKGARVLALCSEITTCMFHGPTESHLD
SMVGQALFGDGASAVIVGAEPDESAGERPI
YELVSAAQTILPNSEGAIDGHLMETRLTFH
LLKDVPGLISNNIEKSLIEAFTPIGINDWN
SIFWVTHPGGPAILDEVEAKLELKKEKLAI
SRHVLSEYGNMSSASVFFVMDELRKRSLEE
GKSTTGDGLDWGVLFGFGPGLTVEMVVLHS
VENKVKSET
(Morus notabilis)
SEQ ID NO: 51
MSMTPSVHEIRKAQRSEGPATVLSIGTATP
TNFVSQADYPDYYFRITNSDHMTDLKDKFK
RMCEKSMITKRHMYLTEEILKENPKMCEYM
APSLDARQDIVVVEVPKLGKEAAAKAIKEW
GQPKSKITHLIFCTTSGVDMPGADYQLTKL
LGLRPSVKRFMMYQQGCFAGGTVLRLAKDL
AENNKGARVLVVCSEITAVTFRGPSHTHLD
SLVGQALFGDGAAAVIVGADPDTSVERPIF
ELVSAAQTILPDSEGAIDGHLREVGLTFHL
LKDVPGLISKNIEKSLVEAFTPIGISDWNS
IFWIAHPGGPAILDQVETKLGLKQEKLSAT
RHVLSEYGNMSSACVLFILDEMRKKSVEEG
KATTGEGLEWGVLFGFGPGLTVETVVLHSL
PAV
OAC
(Cannabis sativa)
SEQ ID NO: 52
MAVKHLIVLKFKDEITEAQKEEFFKTYVNL
VNIIPAMKDVYWGKDVTQKNKEEGYTHIVE
VTFESVETIQDYIIHPAHVGFGDVYRSFWE
KLLIFDYTPRK
(Cannabis sativa)
SEQ ID NO: 53
MAVKHLIVLKFKDEITEAQKEEFFKTYVNL
VNIIPAMKDVYWGKDVTQKKEEGYTHIVEV
TFESVETIQDYIIHPAHVGFGDVYRSFWEK
LLIFDYTPRKLKPK
(Beauveria bassiana)
SEQ ID NO: 54
MAPVTHIVLFEFKPDVTKAQRDEFSAEMLG
LKDKCIHAKTQKPYILRSSGGIDNSIEGLQ
HGITHAFVVEFASVEDRQYYVKEDPAHIAF
VNKLFPFLAKPYIIDFTPGEFN
(Cordyceps brongniartii RCEF 3172)
SEQ ID NO: 55
MAPVTHIVLFEFKPEVTKAQRDEFSAEMLG
LKDKCIHSKTQKPYILRSSGGIDNSIEGLQ
HGITHAFVVEFASVEDRQYYVKEDPAHIAF
VNKLFPSLAKPYIIDFTPGEFN
(Cordyceps confragosa RCEF 1005)
SEQ ID NO: 56
MAPITHVVLFEFKPEVDKAERDELSAEMLG
LKDKCLHATTQKPYIIRSSGGIDNSIEGMQ
HGVTHAFVVEFASAEDRQYYVKEDPVHIAF
VKKVFPRLAKPYIIDFTPGEFN
(Cordyceps fumosorosea ARSEF 2679)
SEQ ID NO: 57
MAPVTHIVMFEFKPEVTKAQRDEFSAEMLD
LKNKCIHPKTNQAYILRSTGGIDNSIEGFQ
HGISHAFVVEFASPEDREYYVKEDPAHLAF
VQKLFPSLAKPYVVDFTPGEFN
(Cordyceps militaris CM01)
SEQ ID NO: 58
MAPITHIVMFEFKSDVTKAQRDELSKEMLA
LKDNCIHAATQKPYIVHSHGGIDNSIEGFQ
HGISHVFVVEFASVEDRTYYVKEDPVHSRY
VQKLLPFLVKPTVVDFTPGEFH
(Torrubiella hemipterigena)
SEQ ID NO: 59
MAPVIHIVMFQFKEDVSTETIKEMSDRMLG
LKTNCIHATTKQPYILSSRGGTDMSIEGLT
QGYTHAYVVEFASKEDRDYYVKEDPVHAAY
VKDVVPLLIKPCIFDYHPGEFTHTKL
CBGAS/CBGVAS
(Cannabis sativa)
SEQ ID NO: 60
MGLSSVCTFSFQTNYHTLLNPHNNNPKTSL
LCYRHPKTPIKYSYNNFPSKHCSTKSFHLQ
NKCSESLSIAKNSIRAATTNQTEPPESDNH
SVATKILNFGKACWKLQRPYTIIAFTSCAC
GLFGKELLHNTNLISWSLMFKAFFFLVAIL
CIASFITTINQIYDLHIDRINKPDLPLASG
EISVNTAWIMSIIVALFGLIITIKMKGGPL
YIFGYCFGIFGGIVYSVPPFRWKQNPSTAF
LLNFLAHIITNFTFYYASRAALGLPFELRP
SFTFLLAFMKSMGSALALIKDASDVEGDTK
FGISTLASKYGSRNLTLFCSGIVLLSYVAA
ILAGIIWPQAFNSNVMLLSHAILAFWLILQ
TRDFALTNYDPEAGRRFYEFMWKLYYAEYL
VYVFI
(Humulus lupulus)
SEQ ID NO: 61
MELSSVSSFSLGTNPFISIPHNNNNLKVSS
YCCKSKSRVINSTNSKHCSPNNNTSNKTTH
LLGLYGQSRCLLKPLSFISCNDQRGNSIRA
SAQIEDRPPESGNLSALTNVKDFVSVCWEY
VRPYTAKGVIICSSCLFGRELLENPNLFSW
PLIFRALLGMLAILGSCFYTAGINQIFDMD
IDRINKPDLPLVSGRISVESAWLLTLSPAI
IGFILILKLNSGPLLTSLYCLAILSGTIYS
VPPFRWKKNPITAFLCILMIHAGLNFSVYY
ASRAALGLAFANSPSFSFITAFITFMTLTL
ASSKDLSDINGDRKFGVETFATKLGAKNIT
LLGTGLLLLNYVAAISTAIIWPKAFKSNIM
LLSHAILAFSLIFQARELDRTNYTPEACKS
FYEFIWILFSAEYVVYLFI
(Saccharomyces cerevisiae)
SEQ ID NO: 62
MASEKEIRRERFLNVFPKLVEELNASLLAY
GMPKEACDWYAHSLNYNTPGGKLNRGLSVV
DTYAILSNKTVEQLGQEEYEKVAILGWCIE
LLQAYFLVADDMMDKSITRRGQPCWYKVPE
VGEIAINDAFMLEAAIYKLLKSHFRNEKYY
IDITELFHEVTFQTELGQLMDLITAPEDKV
DLSKFSLKKHSFIVTFETAYYSFYLPVALA
MYVAGITDEKDLKQARDVLIPLGEYFQIQD
DYLDCFGTPEQIGKIGTDIQDNKCSWVINK
ALELASAEQRKTLDENYGKKDSVAEAKCKK
IFNDLKIEQLYHEYEESIAKDLKAKISQVD
ESRGFKADVLTAFLNKVYKRSK
(Aspergillus terreus)
SEQ ID NO: 63
MLPPSDSKDPRPWQILSQALGFPNYDQELW
WQNTAETLNRVLEQCDYSVHLQYKYLAFYH
KYILPSLGPFRRPGVEPEYISGLSHGGHPL
EISVKIDKSKTICRLGLQAIGPLAGTARDP
LNSFGDRELLKNLATLLPHVDLRLFDHFNA
QVGLDRAQCAVATTKLIKESHNIVCTSLDL
KDGEVIPKVYFSTIPKGLVTETPLFDLTFA
AIEQMEVYHKDAPLRTALSSLKDFLRPRVP
TDASITPPLTGLIGVDCIDPMLSRLKVYLA
TFRMDLSLIRDYWTLGGLLTDAGTMKGLEM
VETLAKTLKLGDEACETLDAERLPFGINYA
MKPGTAELAPPQIYFPLLGINDGFIADALV
EFFQYMGWEDQANRYKDELKAKFPNVDISQ
TKNVHRWLGVAYSETKGPSMNIYYDVVAGN
VARV
(Streptomyces blastmyceticus)
SEQ ID NO: 64
MESAGPGTGPQPPRTSGDFTPDTGVIAEMT
GRPMRFDSDRYRPTDTYAEVACDKVCRAYE
GLGADGGDRESLLAFLRDLTDPWGELPVGT
PPEDACWVSIDGMPLETSVAWAGRKAGVRL
SLESPRGPAKRRMEDGMALTRRLAGRPGVS
VDPCLRVEDLFTDDDPQGYFTIAHAVAWTP
GGHPRYKIFLNPAVRGREQAAARTEEAMIR
LGLEQPWRALTEHLGGAYGPEHEPAALAMD
LVPGDDFRVQVYLAHSGVSAEAIDAKSAVA
ADHVPGSFARALRGINGADDTPEWKRKPPV
TAFSFGPGRAVPGATLYVPMIPVHGSDAAA
RDRVAAFLRSEGMDAVGYEAVLDAISDRSL
PESHTQNFISYRGGDSPRFSVYLAPGVYRE
A
(Marinactinospora thermotolerans)
SEQ ID NO: 65
MAGDPFVDNGTVSSQRPLRAVPGRYPPGAT
HLDAAVDTLVRCHAALGRAPSEAEAAVCLL
RRLWGRWGNTPVERPGWRSYVAVDGSPFEL
SAAWNGDGPAEVRVTVEATADPPTPEGNQE
AGWEYLRGLSRHPGAATARVLALEDLFRPQ
TPHDRCWIMHGMASRPGADPLFKVYLDPDA
RGAAEAPSVLDEAMDRLGVRAAWQGLRGWL
DEHGGSGRIGSLALDLADTDDARVKVYVQH
AGLDWADIDRQAAVARGHVPGAFSAALEEI
TGTEVPPHKPPVTCFAFHRGVGVPTAATLY
IPMPAGVPESDARRRSAAFMRRSGLDSAAY
LAFLAAATGDGEGVRALQNFVAYRPAAPGG
RPRFACYVAPGLYR
(Pestalotiopsis fici W106-1)
SEQ ID NO: 66
MAISTPSNGVSHVAKPLPNLKEVNKGIETD
SEDRAFWWGALSEPLASLLEANHYTKEVQL
HYLRWFYQWILPALGPRPLDGKPYYGSWIT
HDLSPFEYSLNWKEKSSKQTIRFTIEAVTK
QSGTASDPINQLGAKEFLEAVSKDVPGMDL
TRFNQFLEATNVPNDCVDDAIAKHPAHFPR
SRVWIAFDLEHSGNLMAKSYFLPHWRAIQS
GISANTIIGDTVKECNKADGSSYDGSLNAI
ESYLATFTRPEEAPQMGLLSNDCVAETPGS
RLKVYFRSSADTLAKAKDMYNLGGRLKGPK
MDASLKGISDFWYHLFGLDSSDPASDDKVC
IGNHKCIFVYEMRSSQGSEPDIDVKFHIPM
WQLGKTDGQISELLASWFESHGHPDLASRY
KSDLGTAFPKHNITGKSVGTHTYISITHTP
KTGLYMTMYLSPKLPEFYY
(Streptomyces sp. ONZ306)
SEQ ID NO: 67
MIGIDFLECLVSEGIEAEGLYSAIEESARM
VDAPFSRDKVWPILSAFGGGFSDAGGVIFS
LQAGKDVPEMEYSAQISAEVGDPYAHALAT
GVLNETDHPVSTVLAEIVSLAPTSEHYIDC
GIVGGFKKIYANFPHDQQKVSRLADLPAMP
RAVGANAEFFDRYGLDNVALIGVDYRNKTI
NLYFQAPAETAGNLDPKTVSAMLRETGMST
PSEEMVAYADRAYRIYATLGWDSPEVMRLA
FAPQPRRSIDLAELPARLEPRIEQFMRATP
HKYPGALINATAAKWSKKHEVLDLAAYYQV
SALHLKAIQAEEGQSS
(Streptomyces cinnamonensis)
SEQ ID NO: 68
MMSGTADLAGVYAAVEESAGLLDVSCAREK
VWPILAAFEDVLPTAVIAFRVATNARHEGE
FDCRFTVPGSIDPYAVALDKGLTHRSGHPI
ETLVADVQKHCAVDSYGVDFGVVGGFKKIW
VYFPGGRHESLAHLGEIPSMPPGLAATEGF
FARYGLADKVDLIGVDYASKTMNVYFAASP
EVVSAPTVLAMHREIGLPDPSEQMLDFCSR
AFGVYTTLNWDSSKVERIAYSVKTEDPLEL
SARLGSKVEQFLKSVPYGIDTPKMVYAAVT
AGGEEYYKLQSYYQWRTDSRLNLSYIGGRS
(Streptomyces sp. KO-3988)
SEQ ID NO: 69
MPGTDDVAVDVASVYSAIEKSAGLLDVTAA
REVVWPVLTAFEDVLEQAVIAFRVATNARH
EGDFDVRFTVPEEVDPYAVALSRSLIAKTD
HPVGSLLSDIQQLCSVDTYGVDLGVKSGFK
KVWVYFPAGEHETLARLTGLTSMPGSLAGN
VDFFTRYGLADKVDVIGIDYRSRTMNVYFA
APSECFERETVLAMHRDIGLPSPSEQMFKF
CENSFGLYTTLNWDTMEIERISYGVKTENP
MTFFARLGTKVEHFVKNVPYGVDTQKMVYA
AVTSSGEEYYKLQSYYRWRSVSRLNAAYIA
ARDKEST
(Aspergillus versicolor)
SEQ ID NO: 70
MTAPELRAPAGHPQEPPARSSPAQALSSYH
HFPTSDQERWYQEIGSLCSRFLEAGQYGLH
QQYQFMFFFMHHLIPALGPYPQKWRSTISR
SGLPIEFSLNFQKGSHRLLRIGFEPVNFLS
GSSQDPFNRIPIADLLAQLARLQLRGFDTQ
CFQQLLTRFQLSLDEVRQLPPDDQPLKSQG
AFGFDFNPDGAILVKGYVFPYLKAKAAGVP
VATLIAESVRAIDADRNQFMHAFSLINDYM
QESTGYNEYTFLSCDLVEMSRQRVKIYGAH
TEVTWAKIAEMWTLGGRLIEEPEIMEGLAR
LKQIWSLLQIGEGSRAFKGGFDYGKASATD
QIPSPIIWNYEISPGSSFPVPKFYLPVHGE
NDLRVARSLAQFWDSLGWSEHACAYPDMLQ
QLYPDLDVSRTSRLQSWISYSYTAKKGVYM
SVYFHSQSTYLWEED
(Aspergillus fumigatus Af293)
SEQ ID NO: 71
MSIGAEIDSLVPAPPGLNGTAAGYPAKTQK
ELSNGDFDAHDGLSLAQLTPYDVLTAALPL
PAPASSTGFWWRETGPVMSKLLAKANYPLY
THYKYLMLYHTHILPLLGPRPPLENSTHPS
PSNAPWRSFLTDDFTPLEPSWNVNGNSEAQ
STIRLGIEPIGFEAGAAADPFNQAAVTQFM
HSYEATEVGATLTLFEHFRNDMFVGPETYA
ALRAKIPEGEHTTQSFLAFDLDAGRVTTKA
YFFPILMSLKTGQSTTKVVSDSILHLALKS
EVWGVQTIAAMSVMEAWIGSYGGAAKTEMI
SVDCVNEADSRIKIYVRMPHTSLRKVKEAY
CLGGRLTDENTKEGLKLLDELWRTVFGIDD
EDAELPQNSHRTAGTIFNFELRPGKWFPEP
KVYLPVRHYCESDMQIASRLQTFFGRLGWH
NMEKDYCKHLEDLFPHHPLSSSTGTHTFLS
FSYKKQKGVYMTMYYNLRVYST
(Aspergillus fumigatus)
SEQ ID NO: 72
MDGEMTASPPDISACDTSAVDEQTGQSGQS
QAPIPKDIAYHTLTKALLFPDIDQYQHWHH
VAPMLAKMLVDGKYSIHQQYEYLCLFAQLV
APVLGPYPSPGRDVYRCTLGGNMTVELSQN
FQRSGSTTRIAFEPVRYQASVGHDRFNRTS
VNAFFSQLQLLVKSVNIELHHLLSEHLTLT
AKDERNLNEEQLTKYLTNFQVKTQYVVALD
LRKTGIVAKEYFFPGIKCAATGQTGSNACF
GAIRAVDKDGHLDSLCQLIEAHFQQSKIDD
AFLCCDLVDPAHTRFKVYIADPLVTLARAE
EHWTLGGRLTDEDAAVGLEIIRGLWSELGI
IQGPLEPSAMMEKGLLPIMLNYEMKAGQRL
PKPKLYMPLTGIPETKIARIMTAFFQRHDM
PEQAEVFMENLQAYYEGKNLEEATRYQAWL
SFAYTKEKGPYLSIYYFWPE
(Aspergillus oryzae RIB40)
SEQ ID NO: 73
MSLRNDLDNGRPTKRLESWDIASMWLSDRK
DEIQDWWDFSGPQLATLAHEAGYSTMTQIE
LLLFFRSVVLPRMGRFPDACRPRACAQSRS
ILTYDGSPIEYSWKWNNSANDHPEIRFCVE
PVGDGLCADGIVGGKLRATDEILVQLAKRV
PSTDLEWYHHFRDSFGLGHWTDGPLHEDAG
TWQVRRPRMPVAFEFTPKGIVTKVYFTPPA
TLDDMPSFNMFADVVRPIGDKDTTALDESM
EYLSRDPVGATLRPDVLAIDCISPLKSRIK
LYAGTAMTTFTSAISVLTLGGRIPVTRHSI
DEMWALFRMVLGLHDKFLQDEELPVQNPFQ
PSRAHPEDYYSGLLYYFNLAPGALLPDVKL
YLPVIRYGRSDADIALGLQRFMASRHRGQY
VDGFQRAMEIISQRHKSGNGHRIQTYIACS
FDKDGSLSLTSYLNPGVYFSSETVDV
(Aspergillus terreus NIH2624)
SEQ ID NO: 74
MLPPSDSKDPRPWQILSQALGFPNYDQELW
WQNTAETLNRVLEQCDYSVHLQYKYLAFYH
KYILPSLGPFRRPGVEPEYISGLSHGGHPL
EISVKIDKSKTICRLGLQAIGPLAGTARDP
LNSFGDRELLKNLATLLPHVDLRLFDHFNA
QVGLDRAQCAVATTKLIKESHNIVCTSLDL
KDGEVIPKVYFSTIPKGLVTETPLFDLTFA
AIEQMEVYHKDAPLRTALSSLKDFLRPRVP
TDASITPPLTGLIGVDCIDPMLSRLKVYLA
TFRMDLSLIRDYWTLGGLLKDEGTMKGLEM
VETLAKTLKLGDEACETLDAERLPFGINYA
MKPGTAELAPPQIYFPLLGINDGFIADALV
EFFQYMGWEDQASRYKDELKAKFPNVDISQ
TKNVHRWLGVAYSETKGPSMNIYYDVVAGN
VARV
(Aspergillus fumigatus)
SEQ ID NO: 75
MKAANASSAEAYRVLSRAFRFDNEDQKLWW
HSTAPMFAKMLETANYTTPCQYQYLITYKE
CVIPSLGCYPTNSAPRWLSILTRYGTPFEL
SLNCSNSIVRYTFEPINQHTGTDKDPFNTH
AIWESLQHLLPLEKSIDLEWFRHFKHDLTL
NSEESAFLAHNDRLVGGTIRTQNKLALDLK
DGRFALKTYTYPALKAVVTGKTIHELVFGS
VRRLAVREPRILPPLNMLEEYIRSRGSKST
ASPRLVSCDLTSPAKSRIKIYLLEQMVSLE
AMEDLWTLGGRRRDASTLEGLSLVRELWDL
IQLSPGLKSYPAPYLPLGVIPDERLPLMAN
FTLHQNDPVPEPQVYFTTFGMNDMAVADAL
TTFFERRGWSEMARTYETTLKSYYPHADHD
KLNYLHAYISFSYRDRTPYLSVYLQSFETG
DWAVANLSESKVKCQDAACQPTALPPDLSK
TGVYYSGLH
(Aspergillus fumigatus)
SEQ ID NO: 76
MPPAPPDQKPCHQLQPAPYRALSESILFGS
VDEERWWHSTAPILSRLLISSNYDVDVQYK
YLSLYRHLVLPALGPYPQRDPETGIIATQW
RSGMVLTGLPIEFSNNVARALIRIGVDPVT
ADSGTAQDPFNTTRPKVYLETAARLLPGVD
LTRFYEFETELVITKAEEAVLQANPDLFRS
PWKSQILTAMDLQKSGTVLVKAYFYPQPKS
AVTGRSTEDLLVNAIRKVDREGRFETQLAN
LQRYIERRRRGLHVPGVTADKPPATAADKA
FDACSFFPHFLSTDLVEPGKSRVKFYASER
HVNLQMVEDIWTFGGLRRDPDALRGLELLR
HFWADIQMREGYYTMPRGFCELGKSSAGFE
APMMFHFHLDGSQSPFPDPQMYVCVFGMNS
RKLVEGLTTYRRVGWEEMASHYQGNFLANY
PDEDFEKAAHLCAYVSFAYKNGGAYVTLYN
HSFNPVGDVSFPN
(Aspergillus fischeri NRRL 181)
SEQ ID NO: 77
MSPLSMQTDSVQGTAENKSLETNGTSNDQQ
LPWKVLGKSLGLPTIEQEQYWLNTAPYFNN
LLIQCGYDVHQQYQYLAFYHRHVLPVLGPF
IRSSAEANYISGFSAEGYPMELSVNYQASK
ATVRLGCEPVGEFAGTSQDPMNQFMTREVL
GRLSRLDPTFDLRLFDYFDSQFSLITSEAN
LAASKLIKQRRQSKVIAFDLKDGAIIPKAY
FFLKGKSLASGIPVQDVAFNAIESIAPKQI
ESPLRVLRTFVTKLFSKPTVTSDVFILAVD
CIVPEKSRIKLYVADSQLSLATLREFWTLG
GSVTDSATMKGLEIAEELWRILQYDDAVCS
HSNMDQLPLVVNYELSSGSATPKPQLYLPL
HGRNDEAMANALTKFWDYLGWKGLAAQYKK
DLYANNPCRNLAETTTVQRWVAFSYTESGG
AYLTVYFHAVGGMKGNL
(Xylona heveae TC161)
SEQ ID NO: 78
MAPSMTANYPYSQISEFSKTIATSSDLDPN
FGGGVSFKPSSCGGITTARKPWQILQDALG
FRNEDEHFWWETTASVLGCLLEKAGYDVHL
QYQYLSLYYRYVLPSYGPRPLQPGVPHWKS
FMCDDFSPFEPSWNWDGSKSIIRFSFEPIN
RASGTSADPFNQIKPREVLAEISDISAGLD
TQWYDHFAREFFLPSETASIIRSRLPEGEH
MSQSFLAWDLNGGEASTKAYFFPILRSLET
GRSTRDIVVDAITKLDSEKTSLRPSLTVLE
DYMSSLPTEWQAKYEMIAIDCTDPSKSRIK
IYVRMPSMAFNKVRDMYCLGGRLHGPNVDA
AMKILDDLWPRVLYIPEGTGPDDELPSNTH
RTAGAIFNFELKPGNPLPDPKLYLPVRHYA
KSDLDIARGLQSFFRLQGWDEMADSYVEDL
KNIFPTHDLANTAGSHTYLSYSYKKKTGAA
VTMYYNPRIYECPPVVDEVF
(Penicillium polonicum)
SEQ ID NO: 79
MTYSTATPKDSTPVSLLSLYLTFRSKDDKL
WWDNTAPVIGGFLAAAHYKVASQFEFLLFY
HKYILPSLGHYPSPENEGDRWKSFLYRRGE
PLELSFNYQKDSNCTVRLALEPVGPNAGTK
DDPLNEFEAKILVEKIAQLDSNIDLQWVDF
LDKEILLHNDELSQIKNTELEGSAHMSQRL
VGVDFMSGGMKIKPYFVPWLKSLVTGVPTL
QLMFQAIRKLDSVGSFSNGLSEVEAYLAST
DQLLWSEENYLSFDCVDPGKSRIKLYVAEK
VTCFNRIQSHWTLGGQLRSQANQEGLLLLK
KLWNLLGYPGDPAQQTDRYLPFNFNWELRP
SNPIPLPKVYFALGNEPDSLVSKALIGLFT
ELGWSDQIHAHKRSVEFAFPDCNLEETTHV
LTWITVTYEEEKGAYITTYCNAIGGGHKLQ
FR
(Aspergillus taichungensis)
SEQ ID NO: 80
MLLSRTTSSQNPFHLLLSGTPRLPKMRPEQ
EPSIQAPSKKVPLPIADGDARPWQVLSLLL
PFHNPDQKLWWDKVGPLIETYLNCSGYNVG
AQYRYLLMLHSIILPVLGPFPNSTRTHTSW
PYFMNNGDPCDLSINYQGGSAPCVRLGIEP
IGPMAGTNQDPMNEYAGRRLLEDLSRIQPG
IDFQLFDHFRDTLTLSNYKARLCWHAVQEH
GIKAQGHVALDLHEHSFKVKAYSIPLLRSL
TSGVHYVRMMIDSIKMISRDQAITIGLSKV
DEYLAATKHLLVDSRSCFSFDCADLQHSRY
KIYVGANVKSLGEAYDFWTLGGRLKGEAID
RGFQLMETIWKTMYARSLPDRKPREYIPFI
WNWEVSPTDSDPIPKAYFLVLNDYDILVSE
VINCLFGELGWTEHAMTHQIIQKMAYPNHD
FGSSTEIYSWISLAYSQSKGPYITIYSNPA
ASL
(Trypanosoma grayi)
SEQ ID NO: 81
MQLREELRDAVCVFYLVLRALDTVEDDMSL
AVDLKLRELPVFHEHLRDPSWRMCGVGAGR
ERELLERFPHVTRVYARLGKAYQDVITDIC
ARMASGMCEFLTRRVESRADYDLYCHYVAG
LVGHGLTRLYVSGGFEDPNLADDLTNANHM
GLFLQKTNIIRDFYEDICESPPRIFWPREI
WAQYTDDLHAFKEEAHEAKALECLNAMVAD
ALVHVPHVIEYMAALRDPSVFAFCAIPQLM
AMATLALVFNNRNVFHSKVKLTRGSTCSII
LYSTQLQSAMQTMRTQAQNLLARTGPDDVC
YDKIAELVGEAVRAVDAHLQPETDGVARSM
LTRYPALGGRLLYTLIDNVVGYLGK
(Cutaneotrichosporon oleaginosum)
SEQ ID NO: 82
MATLYPSIQSLQKFPYPGDGVVSSTLTDQH
DTEGLIADVLDEQPPAHVPRLGLQNATTTL
DSVNHLKFIQGAMMSLPSGFVGLDASRPWL
VFWTVHSLDLLGVLLPQNIRDRAVSTILHF
LHPTGGFCGGAANTHMPHLLPTYASVVSLA
IVGNAGKGGGWERLVDARQDIYNFFMRCKR
PDGGFVVGDNCEVDVRGTYCLLVVATLLDI
ITPELLHNVDKAIAAGQTFEGGFACSSFTF
KDGNRVAMSEAHGGYTSCSVFSHFLLSSVQ
PPRRLESLPESFPVPIDVDSVVRWSAMMQG
EAADGGGFRGRSNKLVDGCYSWWVGGTFPV
LEELRRREAEVKTSPNGPTATKIVAVDDDG
EDEWADEASMHALFNRGMCDSEVRLMAVAL
QEYTLLVAQSVTRGGLRDKPGKGPDLYHTC
NNLSGLSVAQHRLTHTPEEVQKQREAFKAD
RGLPAVKPTTPGGGWKSEEERQAARREVWA
NVRAWVEDESDTLVVGGQMSQVNTTVPPFN
MLEVRLQPFIDYFYCQ
(Salpingoeca rosetta)
SEQ ID NO: 83
MGYDGLVKLDPEQHLPYVTGGLGTLPSGFE
TLDASRPWLVYWSLNALVILGGTISPELKR
RVINTLRMCQAETGGFGGGVGQVAHAAPTY
AAVNALAIIGTEEAWSIINREKLASWLSSL
IEDDGSMHMHDDGEIDVRAVYCGASAARLC
GLDVDTIFAKCPQWVARCQTYEGGFAAIPG
LEAHGGYTFCGFAAMSILCSTHLIDIPRLT
EWLANRQMPMSGGFQGRPNKLVDGCYSFWV
GGCFPILADLLEAQGLPGDVVNAEALIDYV
VCVCQCPSGFRDKPGKRQDYYHTSYCLSGL
ASMKRFAPNHPILSQLNATHPIHNVPPANA
ERMIQAMSSQTTTRH
(Streptomyces sp. Strain CL190)
SEQ ID NO: 84
MSEAADVERVYAAMEEAAGLLGVACARDKI
YPLLSTFQDTLVEGGSVVVFSMASGRHSTE
LDFSISVPTSHGDPYATVVEKGLFPATGHP
VDDLLADTQKHLPVSMFAIDGEVTGGFKKT
YAFFPTDNMPGVAELSAIPSMPPAVAENAE
LFARYGLDKVQMTSMDYKKRQVNLYFSELS
AQTLEAESVLALVRELGLHVPNELGLKFCK
RSFSVYPTLNWETGKIDRLCFAVISNDPTL
VPSSDEGDIEKFHNYATKAPYAYVGEKRTL
VYGLTLSPKEEYYKLGAYYHITDVQRGLLK
AFDSLED
(Streptomyces sp. Act143)
SEQ ID NO: 85
MSGAADVERVYAAMEEAAGLLGVTCAREKI
YPLLTEFQDTLTDGVVVFSMASGRRSTELD
FSISVPTSQGDPYATVVEKGLFPATGHPVD
DLLADTQKHLPVSMFAIDGEVTGGFKKTYA
FFPTDDMPGVAQLSAIPSMPSSVAENAELF
ARYGLDKVQMTSMDYKKRQVNLYFSELSEQ
TLAPESVLALVRELGLHVPTELGLEFCKRS
FSVYPTLNWDTGKIDRLCFAVISTDPTLVP
STDERDIEQFRAYGTKAPYAYVGEKRTLVY
GLTLSPTEEYYKLGAYYHITDIQRRLLKAF
DALED
(Streptomyces antibioticus)
SEQ ID NO: 86
MTSRVCSTSQRQSILQRGSRPMAEAEARTD
RQDRSVEVCMSGAADVERVYAAMEEAAGLL
GVTCAREKIYPLLTEFQDTLTDGVVVFSMA
SGRRSTELDFSISVPTSQGDPYATVVDKGL
FPATGHPVDDLLADTQKHLPVSMFAIDGEV
TGGFKKTYAFFPTDDMPGVAQLSAIPSMPS
SVAENAELFARYGLDKVQMTSMDYKKRQVN
LYFSELSEQTLAPESVLALVRELGLHVPTE
LGLEFCKRSFSVYPTLNWDTGKIDRLCFAV
ISTDPTLVPSTDERDIEQFRHYGTKAPYAY
VGENRTLVYGLTLSPTEEYYKLGAYYHITD
IQRRLLKAFDALED
(Streptomyces antibioticus)
SEQ ID NO: 87
MSGAADVERVYAAMEEAAGLLGVTCAREKI
YPLLTEFQDTLTDGVVVFSMASGRRSTELD
FSISVPTSQGDPYATVVDKGLFPATGHPVD
DLLADTQKHLPVSMFAIDGEVTGGFKKTYA
FFPTDDMPGVAQLSAIPSMPSSVAENAELF
ARYGLDKVQMTSMDYKKRQVNLYFSELSEQ
TLAPESVLALVRELGLHVPTELGLEFCKRS
FSVYPTLNWDTGKIDRLCFAVISTDPTLVP
STDERDIEQFRHYGTKAPYAYVGENRTLVY
GLTLSPTEEYYKLGAYYHITDIQRRLLKAF
DALED
(Actinobacteria bacterium OV320)
SEQ ID NO: 88
MEVSMSGAADVERVYAAMEEAAGLLDVSCA
REKIYPLLTVFQDTLTDGVVVFSMASGRRS
TELDFSISVPVSQGDPYATVVREGLFRATG
SPVDELLADTVKHLPVSMFAIDGEVTGGFK
KTYAFFPTDDMPGVAQLTGIPSMPASVAEN
AELFARYGLDKVQMTSMDYKKRQVNLYFSD
LKQEYLQPEAVVALARELGLQVPGELGLEF
CKRSFAVYPTLNWDTGKIDRLCFAAISTDP
TLVPSTDERDIEMFREYATKAPYAYVGEKR
TLVYGLTLSPTEEYYKLGAYYHITDIQRQL
LKAFDALED
(Streptomyces sp. Root1310)
SEQ ID NO: 89
MEVSMSGAADVERVYAAMEEAAGLLDVSCA
REKIYPLLTVFQDTLTDGVVVFSMASGRRS
TELDFSISVPVSQGDPYATVVKEGLFQATG
SPVDELLADTVAHLPVSMFAIDGEVTGGFK
KTYAFFPTDDMPGVAQLAAIPSMPASVAEN
AELFARYGLDKVQMTSMDYKKRQVNLYFSD
LKQEYLQPESVVALARELGLRVPGELGLEF
CKRSFAVYPTLNWDTGKIDRLCFAAISTDP
TLVPSEDERDIEMFRNYATKAPYAYVGEKR
TLVYGLTLSSTEEYYKLGAYYHITDIQRQL
LKAFDALED
(Streptomyces sp. Root1310)
SEQ ID NO: 90
MSGAADVERVYAAMEEAAGLLDVSCAREKI
YPLLTVFQDTLTDGVVVFSMASGRRSTELD
FSISVPVSQGDPYATVVKEGLFQATGSPVD
ELLADTVAHLPVSMFAIDGEVTGGFKKTYA
FFPTDDMPGVAQLAAIPSMPASVAENAELF
ARYGLDKVQMTSMDYKKRQVNLYFSDLKQE
YLQPESVVALARELGLRVPGELGLEFCKRS
FAVYPTLNWDTGKIDRLCFAAISTDPTLVP
SEDERDIEMFRNYATKAPYAYVGEKRTLVY
GLTLSSTEEYYKLGAYYHITDIQRQLLKAF
DALED
(Actinobacteria bacterium OV320)
SEQ ID NO: 91
MSGAADVERVYAAMEEAAGLLDVSCAREKT
YPLLTVFQDTLTDGVVVFSMASGRRSTELD
FSISVPVSQGDPYATVVREGLFRATGSPVD
ELLADTVKHLPVSMFAIDGEVTGGFKKTYA
FFPTDDMPGVAQLTGIPSMPASVAENAELF
ARYGLDKVQMTSMDYKKRQVNLYFSDLKQE
YLQPEAVVALARELGLQVPGELGLEFCKRS
FAVYPTLNWDTGKIDRLCFAAISTDPTLVP
STDERDIEMFREYATKAPYAYVGEKRTLVY
GLTLSPTEEYYKLGAYYHITDIQRQLLKAF
DALED
(Streptomyces tendae)
SEQ ID NO: 92
MSGAADVERVYAAMEEAAGLLDVSCAREKT
YPLLTVFQDTLTDGVVVFSMASGRRSTELD
FSISVPVSQGDPYATVVKEGLFRATGSPVD
ELLADTVKHLPVSMFAIDGEVTGGFKKTYA
FFPTDDMPGVAQLTEIPSMPASVAENAELF
ARYGLDKVQMTSMDYKKRQVNLYFSDLKQE
YLQPEAVVALARELGLQVPGELGLEFCKRS
FAVYPTLNWDTGKIDRLCFAAISTDPTLVP
STDERDIEMFREYATKAPYAYVGEKRTLVY
GLTLSSTEEYYKLGAYYHITDIQRQLLKAF
DALED
(Streptomyces sp. URHA0041)
SEQ ID NO: 93
MSGAAEVERVYSAMEESAGLLDVACSREKI
QPILTAFQDVLADGVIVFSMANGRHATELD
FSISVPAGHGDPYAAALEHGLIPATGHPVG
DLLADTQKALPVSMFAVDGEVTSGFKKTYA
FFPTDDMPGLAQLIDIPSMPPSVAENAELF
GRYGLDKVQMISLDYKKNQVNLYFSNLNPE
FLQPEPVQAMVREMGLQLPADKGLAFAKRS
FAVYPTLSWDSAKIERLCFAVISTDPTLAP
AQEQADLDLFSTYANNAPYAYAGEKRTLVY
GLTLSPSEEYYKLGSYYQISDIQRKLLKAF
DALTD
(Streptomyces paucisporeus)
SEQ ID NO: 94
MSGAAEVERVYSAMEEAAGLLDVACSPEKV
RPILTAFQDVLSDGVIVYSMASGRHATELD
FSISVPADHGDPYTAALAHGLIPETDHPVG
NLLADTQKALPVSMFAVDGEVTGGFKKTYA
FFPTDDMPGLAQLIDIPSMPPSVAENAELF
ARYGLDKVQMTSLDYKRKQVNLYFSNLQPE
FLAPEPVLSMVREMGLELPGEKGLKFARRS
FAIYPTLGWESGKIERLCFAVISTDPGLVP
APDEADRALFSTYANNAPYAYAGEKRTLVY
GLTLSPTEEYYKLGSYYQITDIQRTLLKAF
DALTD
CBDAS
(Cannabis sativa)
SEQ ID NO: 95
MKCSTFSFWFVCKIIFFFFSFNIQTSIANP
RENFLKCFSQYIPNNATNLKLVYTQNNPLY
MSVLNSTIHNLRFTSDTTPKPLVIVTPSHV
SHIQGTILCSKKVGLQIRTRSGGHDSEGMS
YISQVPFVIVDLRNMRSIKIDVHSQTAWVE
AGATLGEVYYWVNEKNENLSLAAGYCPTVC
AGGHFGGGGYGPLMRNYGLAADNIIDAHLV
NVHGKVLDRKSMGEDLFWALRGGGAESFGI
IVAWKIRLVAVPKSTMFSVKKIMEIHELVK
LVNKWQNIAYKYDKDLLLMTHFITRNITDN
QGKNKTAIHTYFSSVFLGGVDSLVDLMNKS
FPELGIKKTDCRQLSWIDTIIFYSGVVNYD
TDNFNKEILLDRSAGQNGAFKIKLDYVKKP
IPESVFVQILEKLYEEDIGAGMYALYPYGG
IMDEISESAIPFPHRAGILYELWYICSWEK
QEDNEKHLNWIRNIYNFMTPYVSKNPRLAY
LNYRDLDIGINDPKNPNNYTQARIWGEKYF
GKNFDRLVKVKTLVDPNNFFRNEQSIPPLP
RHRH
(Cannabis sativa)
SEQ ID NO: 96
MKCSTFCFWYVCKIIFFFLSFNIQISIANP
QENFLKCFSQYIPTNVTNAKLVYTQHDQFY
MSILNSTIQNLRFTSDTTPKPLVIITPLNV
SHIQGTILCSKKVGLQIRTRSGGHDAEGMS
YISQVPFVIVDLRNMHSVKIDVHSQTAWVE
AGATLGEVYYWINENNENLSFPAGYCPTVG
AGGHFSGGGYGALMRNYGLAADNIIDAHLV
NVDGKVLDRKSMGEDLFWAIRGGGGENFGI
IAAWKIRLVAVPSMSTIFSVKKNMEIHELV
KLVNKWQNIAYMYEKELLLFTHFITRNITD
NQGKNKTTIHSYFSSIFHGGVDSLVDLMNK
SFPELGIKKTDCKQLSWIDTIIFYSGVVNY
NTTYFKKEILLDRSGGRKAAFSIKLDYVKK
PIPETAMVTILEKLYEEDVGVGMFVFYPYG
GIMDEISESAIPFPHRAGIMYEIWYIASWE
KQEDNEKHINWIRNVYNFTTPYVSQNPRMA
YLNYRDLDLGKTNFESPNNYTQARIWGEKY
FGKNFNRLVKVKTKVDPDNFFRNEQSIPPL
PLRHH
(Cannabis sativa)
SEQ ID NO: 97
MKCSTFCFWYVCKIIFFFLSFNIQISIANP
QENFLKCLSQYIPTNVTNAKLVYTQHDQFY
MSILNSTVQNLRFTSDTTPKPLVITTPLNV
SHIQGTILCSKKVGLQIRTRSGGHDAEGMS
YISQVPFVIVDLRNMHSVKIDVHSQTAWVE
SGATLGEVYYWINENNENLSFPAGYCPTVG
TGGHFSGGGYGALMRNYGLAADNIIDAHLV
NVDGKVLDRKSMGEDLFWAIRGGGGENFGI
IAAWKIRLVAVPSMSTIFSVKKNMEIHELV
KLVNKWQNIAYMYEKELLLFTHFITRNITD
NQGKNKTTIHSYFSSIFHGGVDSLVDLMNK
SFPELGIKKTDCKQLSWIDTIIFYSGVVNY
NTINFKKEILLDRSGGRKAAFSIKLDYVKK
PIPETAMVTILEKLYEEDVGVGMFVFYPYG
GIMDEISESAIPFPHRAGITYEIWYIASWE
KQEDNEKHINWIRNVYNFTTPYVSQNPRMA
YLNYRDLDLGKTNFESPNNYTQARIWGEKY
FGKNFNRLVKVKTKVDPDNFFRNEQSIPPL
PLRHH
CBCAS
(Cannabis sativa)
SEQ ID NO: 98
MNCSTFSFWFVCKIIFFFLSFNIQISIANP
QENFLKCFSEYIPNNPANPKFIYTQHDQLY
MSVLNSTIQNLRFTSDTTPKPLVIVTPSNV
SHIQASILCSKKVGLQIRTRSGGHDAEGLS
YISQVPFAIVDLRNMHTVKVDIHSQTAWVE
AGATLGEVYYWINEMNENFSFPGGYCPTVG
VGGHFSGGGYGALMRNYGLAADNIIDAHLV
NVDGKVLDRKSMGEDLFWAIRGGGGENFGI
IAACKIKLVVVPSKATIFSVKKNMEIHGLV
KLFNKWQNIAYKYDKDLMLTTHFRTRNITD
NHGKNKTTVHGYFSSIFLGGVDSLVDLMNK
SFPELGIKKTDCKELSWIDTTIFYSGVVNY
NTANFKKEILLDRSAGKKTAFSIKLDYVKK
LIPETAMVKILEKLYEEEVGVGMYVLYPYG
GIMDEISESAIPFPHRAGIMYELWYTATWE
KQEDNEKHINWVRSVYNFTTPYVSQNPRLA
YLNYRDLDLGKINPESPNNYTQARIWGEKY
FGKNFNRLVKVKTKADPNNFFRNEQSIPPL
PPRHH
THCAS
(Cannabis sativa)
SEQ ID NO: 99
MNCSAFSFWFVCKIIFFFLSFHIQISIANP
RENFLKCFSKHIPNNVANPKLVYTQHDQLY
MSILNSTIQNLRFISDTTPKPLVIVTPSNN
SHIQATILCSKKVGLQIRTRSGGHDAEGMS
YISQVPFVVVDLRNMHSIKIDVHSQTAWVE
AGATLGEVYYWINEKNENLSFPGGYCPTVG
VGGHFSGGGYGALMRNYGLAADNIIDAHLV
NVDGKVLDRKSMGEDLFWAIRGGGGENFGI
IAAWKIKLVAVPSKSTIFSVKKNMEIHGLV
KLFNKWQNIAYKYDKDLVLMTHFITKNITD
NHGKNKTTVHGYFSSIFHGGVDSLVDLMNK
SFPELGIKKTDCKEFSWIDTTIFYSGVVNF
NTANFKKEILLDRSAGKKTAFSIKLDYVKK
PIPETAMVKILEKLYEEDVGAGMYVLYPYG
GIMEEISESAIPFPHRAGIMYELWYTASWE
KQEDNEKHINWVRSVYNFTTPYVSQNPRLA
YLNYRDLDLGKTNHASPNNYTQARIWGEKY
FGKNFNRLVKVKTKVDPNNFFRNEQSIPPL
PPHHH
(Actinidia chinensis var. chinensis)
SEQ ID NO: 100
MQKHKNLKTYKMKTPTTLLSFAFVVLFLFS
FSWGALAQNHEDFLQCLSLHSQNSTSITKV
IYTPNNSSYLSVLNFSIKNLRFTSPSTPKP
LVIVTPLDESQIQSTIYCAKTHGMEIRTRS
GGHDFEGLSYISEVSFVILDLINLHSIVVD
SENGTAWVQSGATIGQLYYRIAEKSRNYGF
PAGGCPTVGVGGHFSGGGYGMMLRKYGLAA
DNVVDARIIDVNGNILDRKSMGEDLFWAIR
GGGGASFGVIVAWKINLVVVPSKVTVFTIN
RTLEQNATNLIHKWQSIAHKFPQELLVAIL
IKRVDSSHDNGEDTMQAFFTSLYLGGIDQL
IPLMQESFPELGLTREDCTEMSWIESILYF
AGFPSGSSLDVLLNRTQLSTRYFKAKSDYV
KEPIPLFGWKGIWDLFFKDEGELAEMALIP
YGGKMNEISESSIPFPHRAGNLYKILHMVY
WDEEGAEESEKHISWIRKLYSYMAPYVSKF
PRAAYINYRDLDVGVNNKNGNTSYAQASIW
GMKYFKNNFNRLVHVKTKVDPSNFFKNEQS
IPTLPSWWKKRGN
(Populus trichocarpa)
SEQ ID NO: 101
MTCLKASMLPFLLCLLISFSWVISAHPRED
FLKCLSLHFEDPAAMSNAIHTPYNSSYSSI
LQFSIRNLRFNSSELKPLVIVTPTNASHIQ
AAILCSQRHNLQIRIRSGGHDFEGLSYMAA
LPFVIIDLISLRAVNVDATSRTAWVQAGAT
LGELYYSISEKSRTLAFPAGSCPTIGVGGH
FSGGGHGTMVRKFGLASDNVIDAHLIDSKG
RILDRASMGEDLFWAIRGGGGQSFGVVVAW
KISLVEVPSTVTMFSVSRTLEQNATKLLHR
WQYVANTLPEDLVIDVQVTRVNSSQEGNTT
IQATFFSLFLGEVDQLLPVMQESFPELGLV
KDDCFEMSWIESVFYIGGFTSNASLDVLLN
RTPRSIPRFKAKSDYVKEPMPEIAFEGIWE
RFFEEDIEAPTLILIPYGGKMDEISESSTP
FPHRAGNLYVLVSSVSWREESKEASRRHMA
WIRRLYSYLTKYVSKNPREAYVNYRDLDLG
INNLTGTTSYKQASIWGRKYFKNNFDRLVR
VKTEVDPTNFFRNEQSIPSLSSW

What is claimed is:

1. A microbial cell for producing one or more cannabinoids, the microbial cell expressing a cannabinoid biosynthetic pathway comprising a heterologous prenyltransferase enzyme having cannabigerolic acid synthase (CBGAS) or cannabigerovarinic acid synthase (CBGVAS) activity,
the microbial cell further comprising one or more modifications that increases carbon flux to geranyl diphosphate (GPP) and/or carbon flux to one or more of hexanoic acid, hexanoyl-CoA, butyric acid, butyryl-CoA, and/or acetyl-CoA; and/or
the microbial cell produces the cannabinoid from one or more fed precursors selected from olivetol, olivetolic acid, divarin, divarinic acid, hexanoic acid, butyric acid, hexanoyl-CoA, butyryl-CoA, or derivative thereof and/or GPP precursor.

2. The microbial cell of claim 1, wherein the CBGAS or CBGVAS enzyme comprises the amino acid sequence of SEQ ID NO: 60, or a derivative thereof.

3. The microbial cell of claim 1, wherein the CBGAS or CBGVAS comprises an amino acid sequence selected from SEQ ID NO: 60 to 94, or a derivative thereof.

4. The microbial cell of claim 3, wherein the CBGAS comprises an amino acid sequence selected from: SEQ ID NOs: 63, 74, 77, 84-91, 93 and a derivative thereof.

5. The microbial cell of claim 4, wherein the derivative comprises the amino acid sequence of SEQ ID NO: 84 comprising a G286S mutation.

6. The microbial cell of claims 1 to 5, wherein the microbial cell produces GPP from isopentenyl pyrophosphate (IPP) and/or dimethylallyl pyrophosphate (DMAPP).

7. The microbial cell of claim 6, wherein the microbial cell expresses one or more enzymes for converting fed isoprenol and/or prenol to isopentenyl pyrophosphate (IPP) and/or dimethylallyl pyrophosphate (DMAPP) and where the one or more enzymes are optionally kinases.

8. The microbial cell of any one of claims 1 to 7, wherein the microbial cell comprises one or more modifications that increases carbon flux to geranyl diphosphate (GPP), hexanoic acid, hexanoyl-CoA, butyric Acid, butyryl-CoA, and/or acetyl-CoA.

9. The microbial cell of claim 8, wherein the microbial cell comprises genetic modifications to increase carbon flux to (a) both GPP and Hexanoic Acid or Hexanoyl-CoA; or (b) both GPP and Butyric Acid or Butyryl-CoA.

10. The microbial cell of claim 8 or 9, wherein the cannabinoid is a C5 cannabinoid or a C3 cannabinoid, optionally selected from tetrahydrocannabinolic acid (THCA), cannabidiolic acid (CBDA), cannabichromenic acid (CBCA), tetrahydrocannabivarinic acid (THCVA), cannabidovarinic acid (CBDVA), and cannabichrovarinic acid (CNCVA).

11. The microbial cell of claim 10, wherein the biosynthetic pathway comprises Olivetol Synthase (OLS) and Olivetolic Acid Cyclase (OAC) enzymes.

12. The microbial cell of claim 10, wherein the biosynthetic pathway comprises Divarin Synthase (DS) and Divarinic Acid Cyclase (DAC) enzymes.

13. The microbial cell of claim 10 or 11, wherein the biosynthetic pathway comprises a heterologous olivetolic acid cyclase (OAC) enzyme.

14. The microbial cell of claim 13, wherein the OAC comprises the amino acid sequence of SEQ ID NO: 52, or a derivative thereof.

15. The microbial cell of claim 13, wherein the OAC comprises an amino acid sequence selected from SEQ ID NO: 52-59, or a derivative thereof.

16. The microbial cell of claim 10 or 11, wherein the biosynthetic pathway comprises a heterologous olivetol synthase (OLS) enzyme.

17. The microbial cell of claim 16, wherein the OLS comprises the amino acid sequence of SEQ ID NO: 49, or a derivative thereof.

18. The microbial cell of claim 16, wherein the OLS comprises an amino acid sequence selected from SEQ ID NO: 49-51, or a derivative thereof.

19. The microbial cell of any one of claims 8 to 18, wherein the biosynthetic pathway comprises a recombinant acyl-activating enzyme (AAE) that is a hexanoyl-CoA synthase.

20. The microbial cell of claim 19, wherein the AAE comprises the amino acid sequence of SEQ ID NO: 26 or SEQ ID NO: 27, or a derivative thereof.

21. The microbial cell of claim 19, wherein the AAE comprises an amino acid sequence selected from SEQ ID NO: 26 to 48, or a derivative thereof.

22. The microbial cell of any one of claims 8 to 21, wherein the biosynthetic pathway comprises an enzyme selected from Cannabidiolic Acid Synthase (CBDAS), Cannabichromic Acid Synthase (CBCAS), and a Tetrahydrocannabinolic Acid Synthase (THCAS).

23. The microbial cell of any one of claims 8 to 22, wherein the biosynthetic pathway comprises a heterologous tetrahydrocannabinolic acid synthase (THCAS) enzyme.

24. The microbial cell of claim 23, wherein the THCAS comprises the amino acid sequence of SEQ ID NO: 99, or a derivative thereof.

25. The microbial cell of claim 23, wherein the THCAS comprises an amino acid sequence selected from SEQ ID NOS: 99-101, or a derivative thereof.

26. The microbial cell of any one of claims 8 to 22, wherein the biosynthetic pathway comprises a heterologous cannabichromic acid synthase (CBCAS) enzyme.

27. The microbial cell of claim 26, wherein the CBCAS comprises the amino acid sequence of SEQ ID NO: 98, or a derivative thereof.

28. The microbial cell of any one of claims 8 to 22, wherein the biosynthetic pathway comprises a heterologous cannabidiolic acid synthase (CBDAS) enzyme.

29. The microbial cell of claim 28, wherein the CBDAS enzyme comprises the amino acid sequence of SEQ ID NO: 95, or a derivative thereof.

30. The microbial cell of claim 28, wherein the CBDAS enzyme comprises an amino acid sequence selected from SEQ ID NO: 95 to 97, or a derivative thereof.

31. The microbial cell of any one of claims 8 to 30, wherein the cell overexpresses a geranyl diphosphate synthase (GPPS) enzyme.

32. The microbial cell of claim 31, wherein the microbial host cell overexpresses one or more enzymes in the methylerythritol phosphate (MEP) or the mevalonic acid (MVA) pathway.

33. The microbial cell of claim 32, wherein the microbial cell is a bacterium, and overexpresses one or more enzymes in the MEP pathway.

34. The microbial cell of claim 33, wherein the bacterium is selected from Escherichia spp., Bacillus spp., Corynebacterium spp., Rhodobacter spp., Zymomonas spp., Vibrio spp., Pseudomonas spp., Agrobacterium spp., Brevibacterium spp., and Paracoccus spp.

35. The microbial cell of claim 34, wherein the bacterium is selected from Escherichia coli, Bacillus subtilis, Corynebacterium glutamicum, Rhodobacter capsulatus, Rhodobacter sphaeroides, Zymomonas mobilis, Vibrio natriegens, or Pseudomonas putida.

36. The microbial cell of claim 32, wherein the microbial cell is a yeast, and overexpresses one or more enzymes of the MVA pathway.

37. The microbial cell of claim 36, wherein the yeast is selected from Yarrowia spp., Saccharomyces spp., and Pichia spp.

38. The microbial cell of claim 37, wherein the microbial cell is Saccharomyces cerevisiae or Pichia pastoris.

39. The microbial cell of claim 37, wherein the microbial cell is Yarrowia lipolytica.

40. The microbial cell of any one of claims 36 to 39, comprising one or more genetic modifications that increase acetyl-CoA or malonyl-CoA levels or fluxes.

41. The microbial cell of claim 40, wherein the one or more genetic modifications are selected from modifications that increase the rate of beta-oxidation of lipids and modifications that result in overproduction of one or more subunits of the pyruvate dehydrogenase complex.

42. The microbial cell of claim 41, wherein the one or more genetic modification results in overproduction of one or more of pyruvate decarboxylase (PDC), acetylaldehyde dehydrogenase (ALD), and acetyl-CoA synthase (ACS).

43. The microbial cell of any one of claims 40 to 42, wherein the cell has an overexpression of one or more of Acetyl-CoA Carboxylase, Pyruvate Decarboxylase, Dihydrolipoamide Dehydrogenase, Dihydrolipoamide Acetyltransferase, Malate Dehydrogenase, Acetyl-CoA Synthetase, Pyruvate Dehydrogenase E1 Component Subunit Alpha, ATP-Citrate Lyase Subunit 1, ATP-Citrate Lyase Subunit 2, AMP Deaminase, Acetyl-CoA hydrolase, Putative Pyruvate Decarboxylase 2, Acetyl-CoA Synthetase 1, Acetaldehyde Dehydrogenase 1, Acetaldehyde Dehydrogenase 2, Acetaldehyde Dehydrogenase 3, Acetaldehyde Dehydrogenase 4, Acetaldehyde Dehydrogenase 5, Acetaldehyde Dehydrogenase 6, Pyruvate Dehydrogenase E1 Component Subunit Alpha, Pyruvate Dehydrogenase E1 Component Subunit Beta, peroxin 10, multifunctional β oxidation protein (oxidoreductase and hydro-lyase), primary oleate regulator.

44. The microbial cell of any one of claims 40 to 43, wherein the cell has a deletion or inactivation of one or more of Aspartyl Protease, Protease B Vacuolar, Protease B Vacuolar, Glucose-starch Glucosyltransferase Isoform 1, Glucose-6-phosphate Dehydrogenase, Pyruvate Carboxylase 1, Phosphoenolpyruvate Carboxykinase, Fructose-1,6-bisphosphatase, Mitochondrial Carrier, Mitochondrial Carrier Protein, Alcohol Dehydrogenase 1, Alcohol Dehydrogenase 2, Alcohol Dehydrogenase 3, C1-tetrahydrofolate Synthase, Protein C1-Tetrahydrofolate Synthase Precursor Mitochondrial, Phosphoglucomutase, Glycerol-3-phosphate Dehydrogenase, Fatty Acid Synthase Subunit Alpha, Fatty Acid Synthase Subunit Beta, and phosphatidate phosphatase.

45. A method for producing one or more cannabinoids comprising culturing the microbial cell of any one of claims 8 to 44, and recovering the cannabinoid.

46. The method of claim 45, wherein the microbial cells are cultured with C1, C2, C3, C4, C5, and/or C6 carbon substrates.

47. The method of claim 46, wherein the carbon source is glucose, sucrose, fructose, xylose, and/or glycerol.

48. The method of any one of claims 45 to 47, wherein the microbial cell is fed a terpene or terpene precursor, and which is optionally isoprenol and/or prenol.

49. The method of claim 48, wherein the microbial cell expresses one or more kinases the convert isoprenol and/or prenol to isopentenyl pyrophosphate (IPP) and/or dimethylallyl pyrophosphate (DMAPP).

50. The method of any one of claims 45 to 48, wherein culture conditions are selected from aerobic, microaerobic, and anaerobic.

51. The method of claim 49, wherein the microbial cell is cultured at a temperature between 22° C. and 37° C.

52. The method of any one of claims 45 to 50, wherein the cannabinoid or mixture of cannabinoids is recovered from the microbial cell.

53. The method of any one of claims 45 to 50, wherein the cannabinoid or mixture of cannabinoids is recovered from a cell culture medium.

54. The microbial cell of any one of claims 1 to 5, wherein the microbial cell produces the cannabinoid from one or more fed precursors selected from olivetol, olivetolic acid, divarin, divarinic Acid, hexanoic acid, butyric acid, hexanoyl-CoA, butyryl-CoA, and GPP precursor.

55. The microbial cell of claim 54, wherein the biosynthetic pathway comprises an Olivetolic Acid Cyclase (OAC).

56. The microbial cell of claim 54 or 55, wherein the biosynthetic pathway comprises one or more of a Cannabidiolic Acid Synthase (CBDAS), Cannabichromic Acid Synthase (CBCAS), and a Tetrahydrocannabinolic Acid Synthase (THCAS).

57. The microbial cell of any one of claims 54 to 56, wherein the cannabinoid is a C5 cannabinoid or a C3 cannabinoid, optionally selected from tetrahydrocannabinolic acid (THCA), cannabidiolic acid (CBDA), cannabichromic acid (CBCA), tetrahydrocannabivarinic acid (THCVA), and cannabichrovarinic acid (CNCVA).

58. The microbial cell of claim 57, wherein the biosynthetic pathway comprises a heterologous olivetolic acid cyclase (OAC) enzyme.

59. The microbial cell of claim 58, wherein the OAC comprises the amino acid sequence of SEQ ID NO: 52, or a derivative thereof.

60. The microbial cell of claim 57, wherein the OAC comprises an amino acid sequence selected from SEQ ID NO: 52 to 59, or a derivative thereof.

61. The microbial cell of any one of claims 54 to 60, wherein the biosynthetic pathway comprises a heterologous tetrahydrocannabinolic acid synthase (THCAS) enzyme.

62. The microbial cell of claim 61, wherein the THCAS comprises the amino acid sequence of SEQ ID NO: 99, or a derivative thereof.

63. The microbial cell of claim 61, wherein the THCAS comprises an amino acid sequence selected from SEQ ID NOS: 99 to 101, or a derivative thereof.

64. The microbial cell of any one of claims 54 to 60, wherein the biosynthetic pathway comprises a heterologous cannabichromic acid synthase (CBCAS) enzyme.

65. The microbial cell of claim 64, wherein the CBCAS comprises the amino acid sequence of SEQ ID NO: 98, or a derivative thereof.

66. The microbial cell of any one of claims 54 to 60, wherein the biosynthetic pathway comprises a heterologous cannabidiolic acid synthase (CBDAS) enzyme.

67. The microbial cell of claim 66, wherein the CBDAS comprises the amino acid sequence of SEQ ID NO: 95, or a derivative thereof.

68. The microbial cell of claim 66, wherein the CBDAS comprises an amino acid sequence selected from SEQ ID NO: 95 to 97, or a derivative thereof.

69. The microbial cell of any one of claims 54 to 67, wherein the microbial cell is a bacterium, optionally selected from Escherichia spp., Bacillus spp., Corynebacterium spp., Rhodobacter spp., Zymomonas spp., Vibrio spp., Pseudomonas spp., Agrobacterium spp., Brevibacterium spp., and Paracoccus spp.

70. The microbial cell of claim 69, wherein the bacterium is selected from Escherichia coli, Bacillus subtilis, Corynebacterium glutamicum, Rhodobacter capsulatus, Rhodobacter sphaeroides, Zymomonas mobilis, Vibrio natriegens, and Pseudomonas putida.

71. The microbial cell of any one of claims 54 to 68, wherein the microbial cell is a yeast, optionally selected from Yarrowia spp., Saccharomyces spp., and Pichia spp.

72. The microbial cell of claim 71, wherein the microbial cell is Saccharomyces cerevisiae or Pichia pastoris.

73. The microbial cell of claim 71, wherein the microbial cell is Yarrowia lipolytica.

74. The microbial cell of any one of claims 54 to 73, wherein the microbial cell overexpresses a geranyl diphosphate synthase (GPPS) enzyme.

75. The microbial cell of claim 74, wherein the microbial cell overexpresses one or more enzymes in the methylerythritol phosphate (MEP) or the mevalonic acid (MVA) pathway.

76. The microbial cell of claim 75, wherein the microbial cell is a bacterium, and overexpresses one or more enzymes in the MEP pathway.

77. The microbial cell of claim 75, wherein the microbial cell is a yeast, and overexpresses one or more enzymes in the MVA pathway.

78. A method for producing one or more cannabinoids comprising culturing the microbial cell of any one of claims 54 to 77 in the presence of one or more of olivetol, olivetolic acid, divarin, divarinic acid, hexanoic acid, butyric acid, hexanoyl-CoA, butyryl-CoA, and derivative thereof.

79. The method of claim 78, wherein culture conditions are selected from aerobic, microaerobic, and anaerobic.

80. The method of claim 79, wherein the microbial cell is cultured at a temperature between 22° C. and 37° C.

81. The method of any one of claims 78 to 80, wherein the one or more cannabinoids are recovered from the microbial cell.

82. The method of any one of claims 78 to 80, wherein the cannabinoid or mixture of cannabinoids is recovered from a cell culture medium.

83. The method of any one of claims 78 to 82, wherein the microbial cell is fed a terpene or terpene precursor.
